(19) United States
(12) Patent Application Publication
(10) Pub. No.: US 2005/0214321 A1
(43) Pub. Date: Sep. 29, 2005

(54) Recombinant icosahedral virus like particle production in pseudomonads

(75) Inventors: Lada Rasochova, San Diego, CA (US); Philip Phuoc Dao, San Diego, CA (US)

Correspondence Address: KING & SPALDING LLP, 191 PEACHTREE STREET, N.E., 45TH FLOOR, ATLANTA, GA, 30303-1763, US

(73) Assignee: Dow Global Technologies Inc., Midland, MI (US)

(21) Appl. No.: 11/001,626

(22) Filed: Dec. 1, 2004

Related U.S. Application Data
(60) Provisional application No. 60/525,982, filed on Dec. 1, 2003.

(51) Int. Cl. G01N 33/53; C07H 21/04; C12P 21/06; A61K 48/00; C07K 14/005

(52) U.S. Cl. 424/200.1; 435/69.1; 435/320.1; 435/252.34; 530/350; 536/23.7

(57) ABSTRACT

The present invention provides an improved process for the production of recombinant peptides by fusion of recombinant peptides with icosahedral viral capsids and expression of the fusion in bacterial cells of Pseudomonad origin. The Pseudomonad cells support formation of virus like particles from icosahedral viral capsids in vivo, and allow the inclusion of larger recombinant peptides as monomers or concatamers in the virus like particle. The invention specifically provides cells expressing viral capsid fusions, nucleic acids encoding fusions of toxic proteins with icosahedral viral capsids and processes for manufacture of recombinant proteins.

### CROSS REFERENCE TO RELATED APPLICATIONS

This application claims priority to U.S. Provisional patent application Ser. No. 60/525,982 filed Dec. 1, 2003, entitled “High Efficiency Peptide Production in Pseudomonads.”

### FIELD OF THE INVENTION

The present invention provides an improved process for the production of recombinant peptides. In particular, the present invention provides an improved process for the production or presentation of recombinant peptides in bacterial cells utilizing virus like particles from icosahedral viruses.

### BACKGROUND OF THE INVENTION

The genetic engineering revolution has expanded to the development of recombinant peptides for use as human and animal therapeutics. At present, there are more than 100 biotechnology-derived therapeutics and vaccines approved by the U.S. FDA for medical use and over 1000 additional drugs and vaccines are in various phases of clinical trials. (See M. Rai & H. Padh, (2001) “Expression systems for production of heterologous proteins,” Cur. Science 80(9):1121-1128).

Bacterial, yeast, Dictyostelium discoideum, insect, and mammalian cell expression systems are currently used to produce recombinant peptides, with varying degrees of success. One goal in creating expression systems for the production of heterologous peptides is to provide broad based, flexible, efficient, economic, and practical platforms and processes that can be utilized in commercial, therapeutic, and vaccine applications. For example, for the production of certain peptides, it would be ideal to have an expression system capable of producing, in an efficient and inexpensive manner, large quantities of final, desirable products in vivo in order to eliminate or reduce downstream reassembly costs.

Currently, bacteria are the most widely used expression system for the production of recombinant peptides because of their potential to produce abundant quantities of recombinant peptides. However, bacteria are often limited in their capacities to produce certain types of peptides, requiring the use of alternative, and more expensive, expression systems. For example, bacteria systems are restricted in their capacity to produce monomeric antimicrobial peptides due to the toxicity of such peptides to the bacteria, often leading to the death of the cell upon the expression of the peptide. Because of the inherent disadvantages in terms of the costs and product yields of non-bacterial expression systems, significant time and resources have been spent on trying to improve the capacity of bacterial systems to produce a wide range of commercially and therapeutically useful peptides. While progress has been made in this area, additional processes and platforms for the production of heterologous peptides in bacterial expression systems would be beneficial.

Viruses

One approach for improving peptide production in host cell expression systems is to make use of the properties of replicative viruses to produce recombinant peptides of interest. However, the use of replicative, full length viruses has numerous drawbacks for use in recombinant peptide production strategies. For example, it may be difficult to control recombinant peptide production during fermentation conditions, which may require tight regulation of expression in order to maximize efficiency of the fermentation run. Furthermore, the use of replicative viruses to produce recombinant peptides may result in the imposition of regulatory requirements, which may lead to increased downstream purification steps.

To overcome production issues particularly during fermentation, one area of research has focused on the expression and assembly of viruses in a cell that is not a natural host to the particular virus (a non-tropic host cell). A non-tropic cell is a cell that the virus is incapable of successfully entering due to incompatibility between virus capsids and the host receptor molecules, or an incompatibility between the biochemistry of the virus and the biochemistry of the cell, preventing the virus from completing its life cycle. For example, U.S. Pat. No. 5,869,287 to Price et al. describes a method for synthesizing and assembling, in yeast cells, replicable or infectious viruses containing RNA, where either the viral capsids or the RNA contained within the capsids are from a non-yeast virus species of the Nodaviridae or Bromoviridae. However, this approach does not overcome the potential regulatory hurdles that are associated with protein production in replicative viruses.

Virus Like Particles

Another approach for improving the production of recombinant peptides has been to use virus like particles (VLPs). In general, encapsidated viruses include a protein coat or “capsid” that is assembled to contain the viral nucleic acid. Many viruses have capsids that can be “self-assembled” from the individually expressed capsids, both within the cell the capsid is expressed in (“in vivo assembly”) forming VLPs, and outside of the cell after isolation and purification (“in vitro assembly”). Ideally, capsids are modified to contain a target recombinant peptide, generating a recombinant viral capsid-peptide fusion. The fusion peptide can then be expressed in a cell, and, ideally, assembled in vivo to form recombinant viral or virus-like particles.

This approach has been met with varying success. See, for example, C Marusic et al., J. Virol. 75(18):8434-39 (September 2001) (expression in plants of recombinant, helical potato virus X capsids terminally fused to an antigenic, HIV peptide, with in vivo formation of recombinant virus particles); FR Brennan et al., Vaccine 17(15-16):1846-57 (9 Apr. 1999) (expression in plants of recombinant, icosahedral cowpea mosaic virus or helical potato virus X capsids terminally fused to an antigenic, Staphylococcus aureus peptide, with in vivo formation of recombinant virus particles).

U.S. Pat. No. 5,874,087 to Lomonossoff & Johnson describes production of recombinant plant viruses, in plant cells, where the viral capsids include capsids engineered to contain a biologically active peptide, such as a hormone, growth factor, or antigenic peptide. A virus selected from the genera Comovirus, Tombusvirus, Sobemovirus, and Nepovirus is engineered to contain the exogenous peptide encoding sequence and the entire engineered genome of the virus is expressed to produce the recombinant virus. The exogenous peptide-encoding sequence is inserted within one or more of the capsid surface loop motif-encoding sequences.

Attempts have been made to utilize non-tropic cells to produce particular virus like particles. See, for example, J W Lamb et al., J. Gen. Virol. 77(Pt. 7):1349-58 (July 1996), describing expression in insect cells of recombinant, icosahedral potato leaf roll virus capsids terminally fused to a heptadecapeptide, with in vivo formation of virus-like particles. In certain situations, a non-tropic VLP may be preferable. For instance, a non-tropic viral capsid may be more accommodating to foreign peptide insertion without disrupting the ability to assemble into virus like particles than a native viral capsid. Alternatively, the non-tropic viral capsid may be better characterized and understood than a capsid from a native, tropic virus. In addition, the particular application, such as vaccine production, may not allow for the use of a tropic virus in a particular host cell expression system. U.S. Pat. No. 6,232,099 to Chapman et al. describes the use of rod-shaped viruses to produce foreign proteins connected to viral capsid subunits in plants. Rod-shaped viruses, also classified as helical viruses, such as potato virus X (PVX) have recombinant peptides of interest inserted into the genome of the virus to create recombinant viral capsid-peptide fusions. The recombinant virus is then used to infect a host cell, and the virus actively replicates in the host cell and further infects other cells. Ultimately, the recombinant viral capsid-peptide fusion is purified from the plant host cells.

Use of Virus Like Particles in Bacterial Expression Systems

Because of the potential of fast, efficient, inexpensive, and abundant yields of recombinant peptides, bacteria have been examined as host cells in expression systems for the production of recombinant viral capsid-peptide fusion viral like particles.

Researchers have shown that particular wild-type viral capsids without recombinant peptide inserts can be transgenically expressed in non-tropic enterobacteria. Researchers have also shown that these capsids can be assembled, both in vivo and in vitro, to form virus like particles. See, for example, S J Shire et al., Biochemistry 29(21):5119-26 (29 May 1990) (in vitro assembly of virus-like particles from helical tobacco mosaic virus capsids expressed in E. coli); X Zhao et al., Virology 207(2):486-94 (10 Mar. 1995) (in vitro assembly of virus-like particles from icosahedral cowpea chlorotic mottle virus capsids expressed in E. coli); Y Stram et al., Virus Res. 28(1):29-35 (April 1993) (expression of filamentous potato virus Y capsids in E. coli, with in vivo formation of virus-like particles); J Joseph & H S Savithri, Arch. Virol. 144(9):1679-87 (1999) (expression of filamentous chili pepper vein banding virus capsids in E. coli, with in vivo formation of virus-like particles); D J Hwang et al., Proc. Nat'l Acad. Sci. USA 91(19):9067-71 (13 Sep. 1994) (expression of helical tobacco mosaic virus capsids in E. coli, with in vivo formation of virus-like particles); M Sastri et al., J. Mol. Biol. 272(4):541-52 (3 Oct. 1997) (expression of icosahedral physalis mottle virus capsids in E. coli, with in vivo formation of virus-like particles).

To date, successful expression and in vivo assembly of recombinant viral capsid-peptide fusion particles in a non-tropic bacterial cell has been varied. In general, successful in vivo assembly of these particles has been limited to non-icosahedral virus capsid-target peptide fusion particles. See, for example, MN Jagadish et al., Intervirology 39(1-2):85-92 (1996) (expression in non-plant cells of recombinant, filamentous, non-icosahedral Johnsongrass mosaic virus capsids terminally fused to an antigenic peptide, with in vivo formation of virus-like particles).

The expression of peptides linked to icosahedral capsids has been unsuccessful or of limited utility. For example, V Yusibov et al., J. Gen. Virol. 77(Pt. 4):567-73 (April 1996) described in vitro assembly of virus-like particles from E. coli-expressed, recombinant, icosahedral alfalfa mosaic virus capsids terminally fused to a hexahistidine peptide.

Brumfield et al. unsuccessfully attempted to express as in vivo assembled virus like particles recombinant peptides inserted into an icosahedral capsid. See Brumfield et al., (2004) “Heterologous expression of the modified capsid of Cowpea chlorotic mottle bromovirus results in the assembly of protein cages with altered architectures and functions,” J. Gen. Vir. 85: 1049-1053. The reasons for the observed inability of icosahedral viral capsid-peptide fusion particles to assemble as virus like particles in vivo in E. coli are not well understood. Brumfield at al. associate the failure to assemble to the fact that E. coli produces an insoluble capsid.

Chapman, in U.S. Pat. No. 6,232,099, points out that a limited insertion size is tolerated by icosahedral viruses. Chapman cites WO 92/18618, which limits the size of the recombinant peptide in an icosahedral virus for expression in a plant host cell to 26 amino acids in length, in supporting his assertion. Chapman theorizes that a larger peptide present in the internal insertion site in the capsid of icosahedral viruses may result in disruption of the geometry of the protein and/or its ability to successfully interact with other capsids leading to failure of the chimeric virus to assemble. This reference also describes the use of non-replicative rod-shaped viruses to produce capsid-recombinant peptide fusion peptides in cells that can include E. coli.

Therefore, it is an object of the present invention to provide an improved bacterial expression system for the production of virus like particles, wherein the virus like particle is derived from an icosahedral virus.

It is another object of the present invention to provide bacterial organisms for use as host cells in an improved expression system for the production of virus like particles.

It is still another object of the present invention to provide processes for the improved production of virus like particles in bacteria.

It is yet another object of the present invention to provide novel constructs and nucleic acids for use in an improved bacterial expression system for the production of virus like particles.

### SUMMARY OF THE INVENTION

Icosahedral capsid-recombinant peptide fusion particles assemble into viral like particles or soluble cage structures in vivo when expressed in Pseudomonad organisms. Furthermore, large recombinant peptides or peptide concatamers, greater than 50 amino acids, can be inserted into an icosahedral capsid and assembled in vivo in Pseudomonad organisms.

In one aspect of the present invention, Pseudomonad organisms are provided that include a nucleic acid construct encoding a fusion peptide of an icosahedral capsid and a recombinant peptide. In one specific embodiment of the present invention, the Pseudomonad cell is Pseudomonas fluorescens. In one embodiment the cell produces virus like particles or soluble cage structures.

The virus like particles produced in the cell typically are not capable of infecting the cell. The viral capsid sequence can be derived from a virus not tropic to the cell. In one embodiment, the cell does not include viral proteins other than the desired icosahedral capsid. In one embodiment, the viral capsid is derived from a virus with a tropism to a different family of organisms than the cell. In another embodiment, the viral capsid is derived from a virus with a tropism to a different genus of organisms than the cell. In another embodiment, the viral capsid is derived from a virus with a tropism to a different species of organisms than the cell. In one embodiment of the present invention, the icosahedral capsid is derived from a plant icosahedral virus. In a particular embodiment, the icosahedral capsid is derived from the group selected from Cowpea Mosaic Virus, Cowpea Chlorotic Mottle Virus, and Alfalfa Mosaic Virus.

In one embodiment of the present invention, the recombinant peptide fused to the icosahedral capsid is a therapeutic peptide useful for human or animal treatments. In one particular embodiment, the recombinant peptide is an antigen. In one embodiment, the capsid-recombinant peptide virus like particles can be administered as a vaccine in a human or animal application. In another particular embodiment, the recombinant peptide is a peptide that is toxic to the host cell when in free monomeric form. In a more particular embodiment, the toxic peptide is an antimicrobial peptide.

In one embodiment, the recombinant peptide fused to the icosahedral capsid is at least 7, at least 8, at least, 9, at least 10, at least 12, at least 15, at least 20, at least 25, at least 30, at least 35, at least 40, at least 45, at least 50, at least 55, at least 60, at least 65, at least 75, at least 85, at least 95, at least 99, or at least 100 amino acids.

In one embodiment of the present invention, the recombinant peptide fused to the icosahedral capsid contains at least one monomer of a desired target peptide. In an alternative embodiment, the recombinant peptide contains more than one monomer of a desired target peptide. In certain embodiments, the peptide is composed of at least two, at least 5, at least 10, at least 15 or at least 20 separate monomers that are operably linked as a concatameric peptide to the capsid. In another embodiment, the individual monomers in the concatameric peptide are linked by cleavable linker regions. In still another embodiment, the recombinant peptide is inserted into at least one surface loop of the icosahedral viral capsid. In one embodiment, at least one monomer is inserted into more than one surface loops of the icosahedral viral capsid.

More than one loop of the virus like particle can be modified. In one particular embodiment, the recombinant peptide is expressed on at least two surface loops of the icosahedal virus-like particle. In another embodiment, at least two different peptides are inserted into at least two surface loops of the viral capsid, cage or virus-like particle. In another embodiment, at least three recombinant peptides are inserted into at least three surface loops of the virus-like particle. The recombinant peptides in the surface loops can have the same amino acid sequence. In separate embodiments, the amino acid sequence of the recombinant peptides in the surface loops differ.

In still another embodiment, the cell includes at least one additional nucleic acid encoding a second either wild-type capsid or capsid-recombinant peptide fusion peptide, wherein the multiple capsids are assembled in vivo to produce chimeric virus like particles.

In one aspect of the present invention, Pseudomonad organisms are provided that include a fusion peptide of an icosahedral capsid and a recombinant peptide. In one specific embodiment of the present invention, the Pseudomonad cell is Pseudomonas fluorescens. In one embodiment the capsid-recombinant peptide fusion peptide assembles in vivo to form a virus like particle.

In another aspect of the present invention, nucleic acid constructs are provided encoding a fusion peptide of an icosahedral capsid and a recombinant peptide. In one embodiment of the present invention, the icosahedral capsid is derived from a plant icosahedral virus. In a particular embodiment, the icosahedral capsid is derived from the group selected from Cowpea Mosaic Virus, Cowpea Chlorotic Mottle Virus, and Alfalfa Mosaic Virus.

In one embodiment, the recombinant peptide is a peptide that is toxic to the host cell when in free monomeric form. In a more particular embodiment, the toxic peptide is an antimicrobial peptide.

In one embodiment of the present invention, the recombinant peptide contains at least one monomer of a desired target peptide. In an alternative embodiment, the recombinant peptide contains more than one monomer of a desired target peptide. In still another embodiment, the recombinant peptide is inserted into at least one surface loop of the icosahedral virus capsid.

In another embodiment, the nucleic acid construct can include additional nucleic acid sequences including at least one promoter, at least one selection marker, at least one operator sequence, at least one origin of replication, and at least one ribosome binding site.

In one aspect, the present invention provides a process for producing a recombinant peptide including:
a) providing a Pseudomonad cell;
b) providing a nucleic acid encoding a fusion peptide, wherein the fusion is of a recombinant peptide and an icosahedral capsid;
c) expressing the nucleic acid in the Pseudomonad cell, wherein the expression in the cell provides for in vivo assembly of the fusion peptide into virus like particles; and
d) isolating the virus like particles.

In one embodiment, the process further includes: e) cleaving the fusion product to separate the recombinant peptide from the capsid. In one embodiment of the present invention, the Pseudomonad cell is Pseudomonas fluorescens.

In one embodiment, the process includes co-expressing another nucleic acid encoding a wild-type capsid or capsid-recombinant peptide fusion peptide, wherein the capsids are assembled in vivo to produce chimeric virus like particles.

In another aspect of the present invention, an expression system for the production of recombinant peptides is provided including:
a) a Pseudomonad cell;
b) a nucleic acid encoding a fusion peptide; wherein the fusion peptide comprises at least one recombinant peptide, and at least one icosahedral viral capsid; and
c) a growth medium.

When expressed the fusion peptide can assemble into virus like particles within the cell.

### BRIEF DESCRIPTION OF THE FIGURES

FIG. 1 presents a plasmid map of a CCMV129-CP expression plasmid useful for expression of recombinant VLPs in Pseudomonad host cells.

FIG. 2 illustrates a scheme for production of peptide monomers in Virus-Like Particles (VLP) in host cells, e.g., Pseudomonad host cells. A desired target peptide insert coding sequence (“I”) is inserted, in-frame, into the viral capsid coding sequence (“CP”) in constructing a recombinant viral capsid gene (“rCP”), which, as part of a vector, is transformed into the host cell and expressed to form recombinant capsids (“rCP”). These are then assembled to form VLPs containing up to 180 rCPs each, in the case of CCMV. The VLPs are illustrated with target peptide inserts (“I”) expressed in external loop(s) of the capsid. The assembled VLPs each contain multiple peptide inserts per particle, e.g., up to 180 or a multiple thereof. The VLPs are then readily precipitated from cell lysate for recovery, e.g., by PEG precipitation. The recombinant peptide inserts expressed in the capsid surface loops and/or termini can be isolated in highly pure form from the precipitated VLPs.

FIG. 3 illustrates a scheme for production of peptide multimers in VLPs in host cells, e.g., Pseudomonad host ells. The peptide insert is a multimer (a trimer is shown) of the desired target peptide(s), whose coding sequences (“i”) are inserted into the viral capsid coding sequence (“CP”) in constructing a recombinant viral capsid gene (“rCP”). Each of the target peptide coding sequences is bounded by coding sequences for cleavage sites (“*”) and the entire nucleic acid insert is labeled “I.” In the illustration, only one trimer insertion is made per CCMV capsid, and each of the resulting VLPs contains up to 180 peptide inserts (“I”) for a total of up to 540 target peptides (“i”). The target peptides are then readily isolated in highly pure form, after precipitation of the VLPs, by treatment of the VLPs with a cleavage agent, e.g., an acid or an enzyme.

FIG. 4 is a plasmid Map of CCMV63-CP expression plasmid useful for expression of recombinant VLPs. Restriction sites AscI and NotI were engineered onto CCMV-CP (SEQ ID NO:1) to serve as an insertion site for peptides.

FIG. 5 is a plasmid Map of R26C—CCMV63/129-CP expression plasmid useful for expression of recombinant VLPs. Two insertion sites (AscI-NotI and BamHI) were engineered in the CP for insertions of two identical or different peptides.

FIG. 6 is an image of a SDS-PAGE gel showing expression of chimeric CCMV CP in Pseudomonas fluorescens 24 hours post induction. Chimeric CP has been engineered to express a 20 amino acid antigenic peptide PD1. The chimeric CP has slower mobility compared to the non-engineered wild type (wt) CCMV CP. Lane 1 is a size ladder, lane 2 is wild-type CP 0 hours post-induction, lane 3 is wild-type CP 24 hours post-induction, lane 4 is CCMV129-PD1 0 hours post induction and lane 5 is CCMV129-PD1 24 hours post induction.

FIG. 7 is an image of a western blot showing expression of chimeric CCMV CP in Pseudomonas fluorescens. Chimeric CP has been engineered to express a 20 amino acid antigenic peptide PD1. The chimeric CP has slower mobility compared to the non-engineered wild type (wt) CCMV CP. Lane 1 is a size ladder, lane 2 is wild-type CP 0 hours post-induction, lane 3 is wild-type CP 24 hours post-induction, lane 4 is CCMV129-PD1 0 hours post induction and lane 5 is CCMV129-PD1 24 hours post induction.

FIG. 8 is an image of a western blot of CCMV129-PD1 VLP sucrose gradient fractions. Chimeric CCMV CPs engineered to express a 20 amino acid antigenic peptide PD1 were expressed in Pseudomonas fluorescens. Chimeric VLPs were isolated 24 hours post induction by PEG precipitation and fractionated on sucrose density gradient. The VLP fractions were positive for chimeric CP. Lane 1 is a CCMV129-PD1 VLP sucrose gradient fraction, lane 2 is a CCMV129-PD1 VLP sucrose gradient fraction, lane 3 is a CCMV129-PD1 VLP sucrose gradient fraction and lane 4 is a size ladder.

FIG. 9 is an electron microscopy (EM) image of chimeric CCMV VLPs displaying 20 amino acid antigenic peptides PD1. The VLPs were isolated from P. fluorescens using PEG precipitation and sucrose density fractionation.

FIG. 10 is an image of a SDS-PAGE gel showing expression of chimeric CCMV CP in Pseudomonas fluorescens 12, 24, and 48 hours post induction. Chimeric CP has been engineered to express an antimicrobial peptide D2A21 trimer separated by acid hydrolysis sites. The chimeric CP has slower mobility compared to the non-engineered wild type (wt) CCMV CP. Lane 1 is a size ladder, lane 2 is wild-type CP 0 hours post induction, lane 3 is wild-type CP 12 hours post induction, lane 4 is wild-type CP 24 hours post induction, lane 5 is wild-type CP 48 hours post induction, lane 6 is CCMV129-(D2A21)₃ 0 hours post induction, lane 7 is CCMV129-(D2A21)₃ 12 hours post induction, lane 8 is CCMV129-(D2A21)₃ 24 hours post induction and lane 9 is CCMV129-(D2A21)₃ 48 hours post induction.

FIG. 11 is an image of a western blot of CCMV129-(D2A21)3 VLP sucrose gradient fractions. Chimeric CCMV CPs engineered to express a 96 amino acid antimicrobial peptide D2A21 trimer separated by acid hydrolysis sites were expressed in Pseudomonas fluorescens. Chimeric VLPs were isolated 24 hours post induction by PEG precipitation and fractionated on sucrose density gradient. The VLP fractions were positive for chimeric CP. Lane 1 is a size ladder, lane 2-4 are CCMV129-(D2A21)₃ VLP sucrose gradient fractions.

FIG. 12 is an electron microscopy (EM) image of chimeric CCMV VLPs displaying an antimicrobial peptide D2A21 trimer separated by acid hydrolysis sites. The VLPs were isolated from P. fluorescens using PEG precipitation and sucrose density fractionation.

FIG. 13 is a HPLC chromatogram showing release of AMP D2A21 peptide monomers from chimeric VLPs engineered to display an antimicrobial peptide D2A21 trimer separated by acid cleavage sites by treatment with acid. The AMP peptide peak has not been detected in non-engineered (empty) VLPs.

FIG. 14 is a MALDI-MS graph showing the identity of AMP D2A21 peptide monomers released from chimeric VLPs engineered to display an antimicrobial peptide D2A21 trimer separated by acid cleavage sites by treatment with acid. The molecular weight is as predicted for the D2A21 peptide monomer.

FIG. 15 is an image of a SDS-PAGE gel showing expression of chimeric CCMV CP in Pseudomonas fluorescens 12 and 24 hours post induction. Chimeric CP has been engineered to express four different 25 amino acid antigenic peptides PA1, PA2, PA3, and PA4. The chimeric CP has slower mobility compared to the non-engineered wild type (wt) CCMV CP. Lane 1 is a size ladder, lane 2 is CCMV129-PA1 0 hours post induction, lane 3 is CCMV129-PA1 12 hours post induction, lane 4 is CCMV129-PA1 24 hours post induction, lane 5 is CCMV129-PA2 0 hours post induction, lane 6 is CCMV129-PA2 12 hours post induction, lane 7 is CCMV129-PA2 24 hours post induction, lane 8 is CCMV129-PA3 0 hours post induction, lane 9 is CCMV129-PA3 12 hours post induction, lane 10 is CCMV129-PA3 24 hours post induction, lane 11 is CCMV129-PA4 0 hours post induction, lane 12 is CCMV129-PA4 12 hours post induction, lane 13 is CCMV129-PA4 24 hours post induction.

FIG. 16 is an image of a western blot of CCMV129-PA1, CCMV129-PA2, CCMV129-PA3, CCMV129-PA4 VLP sucrose gradient fractions. Chimeric CCMV CPs engineered to express a 25 amino acid antigenic PA peptides were expressed in Pseudomonas fluorescens. Chimeric VLPs were isolated 24 hours post induction by PEG precipitation and fractionated on sucrose density gradient. The VLP fractions were positive for chimeric CP. Lane 1 is a size ladder, lane 2-4 are CCMV129-PA1 VLP sucrose gradient fractions, lanes 5-7 are CCMV129-PA2 VLP sucrose gradient fractions, lanes 8-10 are CCMV129-PA3 VLP sucrose gradient fractions and lanes 11-13 are CCMV129-PA4 VLP sucrose gradient fractions.

FIG. 17 is an image of a SDS-PAGE showing expression of chimeric CCMV CP in Pseudomonas fluorescens. Chimeric CCMV63-CP has been engineered to express a 20 amino acid antimicrobial peptide PBF20 separated by acid hydrolysis sites. The chimeric CP has slower mobility compared to the non-engineered wild type (wt) CCMV CP. Lane 1 is a size ladder, lane 2 is wild-type CP 0 hours post induction, lane 3 is wild-type CP 24 hours post induction, lane 4 is CCMV63-PBF20 0 hours post induction, lane 5 is CCMV63-PBF20 24 hours post induction.

FIG. 18 is an electron microscopy (EM) image of chimeric CCMV VLPs derived from CCMV63-CP and displaying a 20 amino acid antimicrobial peptide PBF20 separated by acid hydrolysis sites. The chimeric VLPs were isolated from P. fluorescens using PEG precipitation and sucrose density fractionation.

FIG. 19 is an image of a SDS-PAGE showing expression of chimeric CCMV CP in Pseudomonas fluorescens. Chimeric CCMV129-CP has been engineered to express a 20 amino acid antimicrobial peptide PBF20 separated by acid hydrolysis sites. Lane 1 is a size ladder, lane 2 is CCMV129-PBF20 0 hours post induction and lane 3 is CCMV129-PBF20 24 hours post induction.

FIG. 20 is an electron microscopy (EM) image of chimeric CCMV VLPs derived from CCMV129-CP and displaying a 20 amino acid antimicrobial peptide PBF20 separated by acid hydrolysis sites. The chimeric VLPs were isolated from P. fluorescens using PEG precipitation and sucrose density fractionation.

FIG. 21 is an image of a SDS-PAGE showing expression of chimeric CCMV CP in Pseudomonas fluorescens. Chimeric CCMV63/129-CP has been engineered to express a 20 amino acid antimicrobial peptide PBF20 separated by acid hydrolysis sites in two different insertion sites in the CP (63 and 129). Chimeric CP containing a double insert (CP+2×20 AA) has slower mobility on the SDS-PAGE gel compared to the capsid engineered to express a single insert (CP+1×20 AA) of the same peptide. Lane 1 is a size ladder, lane 2 is CCMV63-PBF20 0 hours post induction, lane 3 is CCMV63-PBF20 24 hours post induction, lane 4 is CCMV63/129-2×(PBF20) 0 hours post induction, lane 5 is CCMV63/129-2×(PBF20) 24 hours post induction, lane 6 is CCMV63/129-2×(PBF20) 0 hours post induction, lane 7 is CCMV63/129-2×(PBF20) 24 hours post induction, lane 8 is CCMV63/129-2×(PBF20) 0 hours post induction, lane 9 is CCMV63/129-2×(PBF20) 24 hours post induction.

FIG. 22 is an electron microscopy (EM) image of chimeric CCMV VLPs derived from CCMV63/129-CP displaying a 20 amino acid antimicrobial peptide PBF20 separated by acid hydrolysis sites in two insertion sites per capsid (63 and 129). The chimeric VLPs were isolated from P. fluorescens using PEG precipitation and sucrose density fractionation.

### DETAILED DESCRIPTION

The present invention provides a process for the expression in bacteria of fusion peptides comprising an icosahedral viral capsid and a recombinant peptide of interest. The term “peptide” as used herein is not limited to any particular molecular weight, and can also include proteins or polypeptides. The present invention further provides bacterial cells and nucleic acid constructs for use in the process. Specifically, the invention provides Pseudomonad organisms with nucleic acid construct encoding a fusion peptide of an icosahedral capsid and a recombinant peptide. In one specific embodiment of the present invention, the Pseudomonad cell is Pseudomonas fluorescens. In one embodiment the cell produces virus like particles or soluble cage structures. The invention also provides nucleic acid constructs encoding the fusion peptide of an icosahedral capsid and a recombinant peptide, which can in one embodiment, be a therapeutic peptide useful for human and animal treatments.

The invention also provides a process for producing a recombinant peptide in a Pseudomonad cell by providing: a nucleic acid encoding a fusion peptide of a recombinant peptide and an icosahedral capsid; expressing the nucleic acid in the Pseudomonad cell, wherein the expression in the cell provides for in vivo assembly of the fusion peptide into virus like particles; and isolating the virus like particles.

### I. Recombinant Pseudomonad Cells

The present invention provides Pseudomonad cells that include a nucleic acid construct encoding a fusion peptide of an icosahedral capsid and a recombinant peptide. The cells can be utilized in a process for producing recombinant peptides.

Viral Capsids

In one embodiment, the invention provides Pseudomonad cells for use in a process for producing peptides by expression of the peptide fused to an icosahedral viral capsid. The expression typically results in at least one virus like particle (VLP) in the cell.

Viruses can be classified into those with helical symmetry or icosahedral symmetry. Generally recognized capsid morphologies include: icosahedral (including icosahedral proper, isometric, quasi-isometric, and geminate or “twinned”), polyhedral (including spherical, ovoid, and lemon-shaped), bacilliform (including rhabdo- or bullet-shaped, and fusiform or cigar-shaped), and helical (including rod, cylindrical, and filamentous); any of which may be tailed and/or may contain surface projections, such as spikes or knobs.

Morphology

In one embodiment of the invention, the amino acid sequence of the capsid is selected from the capsids of viruses classified as having any icosahedral morphology. In one embodiment, the capsid amino acid sequence will be selected from the capsids of entities that are icosahedral proper. In another embodiment, the capsid amino acid sequence will be selected from the capsids of icosahedral viruses. In one particular embodiment, the capsid amino acid sequence will be selected from the capsids of icosahedral plant viruses. However, in another embodiment, the viral capsid will be derived from an icosahedral virus not infectious to plants. For example, in one embodiment, the virus is a virus infectious to mammals.

Generally, viral capsids of icosahedral viruses are composed of numerous protein sub-units arranged in icosahedral (cubic) symmetry. Native icosahedral capsids can be built up, for example, with 3 subunits forming each triangular face of a capsid, resulting in 60 subunits forming a complete capsid. Representative of this small viral structure is e.g. bacteriophage ØX174. Many icosahedral virus capsids contain more than 60 subunits. Many capsids of icosahedral viruses contain an antiparallel, eight-stranded beta-barrel folding motif. The motif has a wedge-shaped block with four beta strands (designated BIDG) on one side and four (designated CHEF) on the other. There are also two conserved alpha-helices (designated A and B), one is between betaC and betaD, the other between betaE and betaF.

Enveloped viruses can exit an infected cell without its total destruction by extrusion (budding) of the particle through the membrane, during which the particle becomes coated in a lipid envelope derived from the cell membrane (See, e.g.: A J Cann (ed.) (2001) Principles of Molecular Virology (Academic Press); A Granoff and R G Webster (eds.) (1999) Encyclopedia of Virology (Academic Press); D L D Caspar (1980) Biophys. J. 32:103; DLD Caspar and A Klug (1962) Cold Spring Harbor Symp. Quant. Biol. 27:1; J Grimes et al. (1988) Nature 395:470; J E Johnson (1996) Proc. Nat'l Acad. Sci. USA 93:27; and J Johnson and J Speir (1997) J. Mol. Biol. 269:665).

Viruses

Viral taxonomies recognize the following taxa of encapsidated-particle entities:
Group I Viruses, i.e. the dsDNA viruses;
Group II Viruses, i.e. the ssDNA viruses;
Group IV Viruses, i.e. the dsRNA se viruses;
Group IV Viruses, i.e. the ssRNA (+)-stranded viruses with no DNA stage;
Group V Viruses, i.e. the ssRNA (−)-stranded viruses;
Group VI Viruses, i.e. the RNA retroid viruses, which are ssRNA reverse transcribing viruses;
Group VII Viruses, i.e. the DNA retroid viruses, which are dsDNA reverse transcribing viruses;
Deltaviruses;
Viroids; and
Satellite phages and Satellite viruses, excluding Satellite nucleic acids and Prions.

Members of these taxa are well known to one of ordinary skill in the art and are reviewed in: H. V. Van Regenmortel et al. (eds.), Virus Taxonomy: Seventh Report of the International Committee on Taxonomy of Viruses (2000) (Academic Press/Elsevier, Burlington Mass., USA); the Virus Taxonomy web-page of the University of Leicester (UK) Microbiology & Immunology Department at http://wwwmicro.msb.le.ac.uk/3035/Virusgroups.html; and the on-line “Virus” and “Viroid” sections of the Taxonomy Browser of the National Center for Biotechnology Information (NCBI) of the National Library of Medicine of the National Institutes of Health of the US Department of Health & Human Services (Washington, D.C., USA) at http://www.ncbi.nlm.nih.gov/Taxonomy/tax.html.

The amino acid sequence of the capsid may be selected from the capsids of any members of any of these taxa. Amino acid sequences for capsids of the members of these taxa may be obtained from sources, including, but not limited to, e.g.: the on-line “Nucleotide” (Genbank), “Protein,” and “Structure” sections of the PubMed search facility offered by the NCBI at http://www.ncbi.nlm.nih.gov/entrez/query.fcgi.

In one embodiment, the capsid amino acid sequence will be selected from taxa members that are specific for at least one of the following hosts: fungi including yeasts, plants, protists including algae, invertebrate animals, vertebrate animals, and humans. In one embodiment, the capsid amino acid sequence will be selected from members of any one of the following taxa: Group I, Group II, Group III, Group IV, Group V, Group VII, Viroids, and Satellite Viruses. In one embodiment, the capsid amino acid sequence will be selected from members of any one of these seven taxa that are specific for at least one of the six above-described host types. In a more specific embodiment, the capsid amino acid sequence will be selected from members of any one of Group II, Group III, Group IV, Group VII, and Satellite Viruses; or from any one of Group II, Group IV, Group VII, and Satellite Viruses. In another embodiment, the viral capsid is selected from Group IV or Group VII.

The viral capsid sequence can be derived from a virus not tropic to the cell. In one embodiment, the cell does not include viral proteins from the particular selected virus other than the desired icosahedral capsids. In one embodiment, the viral capsid is derived from a virus with a tropism to a different family of organisms than the cell. In another embodiment, the viral capsid is derived from a virus with a tropism to a different genus of organisms than the cell. In another embodiment, the viral capsid is derived from a virus with a tropism to a different species of organisms than the cell.

In a specific embodiment, the viral capsid is selected from a virus of Group IV.

In one embodiment, the viral capsid is selected form an icosahedral virus. The icosahedral virus can be selected from a member of any of the Papillomaviridae, Totiviridae, Dicistroviridae, Hepadnaviridae, Togaviridiae, Polyomaviridiae, Nodaviridae, Tectiviridae, Leviviridae, Microviridae, Sipoviridae, Nodaviridae, Picornoviridae, Parvoviridae, Calciviridae, Tetraviridae, and Satellite viruses.

In a particular embodiment, the sequence will be selected from members of any one of the taxa that are specific for at least one plant host. In one embodiment the icosahedral plant virus species will be a plant-infectious virus species that is or is a member of any of the Bunyaviridae, Reoviridae, Rhabdoviridae, Luteoviridae, Nanoviridae, Partitiviridae, Sequiviridae, Tymoviridae, Ourmiavirus, Tobacco Necrosis Virus Satellite, Caulimoviridae, Geminiviridae, Comoviridae, Sobemovirus, Tombusviridae, or Bromoviridae taxa. In one embodiment, the icosahedral plant virus species is a plant-infectious virus species that is or is a member of any of the Luteoviridae, Nanoviridae, Partitiviridae, Sequiviridae, Tymoviridae, Ourmiavirus, Tobacco Necrosis Virus Satellite, Caulimoviridae, Geminiviridae, Comoviridae, Sobemovirus, Tombusviridae, or Bromoviridae taxa. In specific embodiments, the icosahedral plant virus species is a plant infectious virus species that is or is a member of any of the Caulimoviridae, Geminiviridae, Comoviridae, Sobemovirus, Tombusviridae, or Bromoviridae. In more particular embodiments, the icosahedral plant virus species will be a plant-infectious virus species that is or is a member of any of the Comoviridae, Sobemovirus, Tombusviridae, or Bromoviridae. In more particular embodiments, the icosahedral plant virus species will be a plant-infectious virus species that is a member of the Comoviridae or Bromoviridae family. In a particular embodiment the viral capsid is derived from a Cowpea Mosaic Virus or a Cowpea Chlorotic Mottle Virus. In another embodiment, the viral capsid is derived from a species of the Bromoviridae taxa. In a specific embodiment, the capsid is derived from an Ilarvirus or an Alfamovirus. In a more specific embodiment, the capsid is derived from a Tobacco streak virus, or an Alfalfa mosaic virus (AMV) (including AMV 1 or AMV 2).

VLP

The icosahedral viral capsid of the invention is non-infective in the host cells described. In one embodiment, a virus like particle (VLP) or cage structure is formed in the host cell during or after expression of the viral capsid. In one embodiment, the VLP or cage structure also includes the peptide of interest, and in a particular embodiment, the peptide of interest is expressed on the surface of the VLP. The expression system typically does not contain additional viral proteins that allow infectivity of the virus. In a typical embodiment, the expression system includes a host cell and a vector which codes for one or more viral capsids and an operably linked peptide of interest. The vector typically does not include additional viral assembly proteins. The invention is derived from the discovery that viral capsids form to a greater extent in certain host cells and allow for more efficient recovery of recombinant peptide.

In one embodiment, the VLP or cage structure is a multimeric assembly of capsids, including from three to about 200 capsids. In one embodiment, the VLP or cage structure includes at least 30, at least 50, at least 60, at least 90 or at least 120 capsids. In another embodiment, each VLP or cage structure includes at least 150 capsids, at least 160, at least 170, or at least 180 capsids.

In one embodiment, the VLP is expressed as an icosahedral structure. In another embodiment, the VLP is expressed in the same geometry as the native virus that the capsid sequence is derived of. In a separate embodiment, however, the VLP does not have the identical geometry of the native virus. In certain embodiments, for example, the structure is produced in a particle formed of multiple capsids but not forming a native-type VLP. For example, a cage structure of as few as 3 viral capsids can be formed. In separate embodiments, cage structures of about 6, 9, 12, 15, 18, 21, 24, 27, 30, 33, 36, 39, 42, 45, 48, 51, 54, 57, or 60 capsids can be formed.

In one embodiment, at least one of the capsids includes at least one peptide of interest. In one embodiment, the peptide is expressed within at least one internal loop, or in at least one external surface loop of the VLP.

More than one loop of the viral capsid can be modified. In one particular embodiment, at the recombinant peptide is expressed on at least two surface loops of the icosahedal virus-like particle. In another embodiment, at least two different peptides are inserted into at least two surface loops of the viral capsid, cage or virus-like particle. In another embodiment, at least three recombinant peptides are inserted into at least three surface loops of the virus-like particle. The recombinant peptides in the surface loops can have the same amino acid sequence. In separate embodiments, the amino acid sequence of the recombinant peptides in the surface loops differs.

In certain embodiments, the host cell can be modified to improve assembly of the VLP. The host cell can, for example, be modified to include chaperone proteins that promote the formation of VLPs from expressed viral capsids. In another embodiment, the host cell is modified to include a repressor protein to more efficiently regulate the expression of the capsid to promote regulated formation of the VLPs.

The nucleic acid sequence encoding the viral capsid or proteins can also be additionally modified to alter the formation of VLPs (see e.g. Brumfield, et al. (2004) J. Gen. Virol. 85: 1049-1053). For example, three general classes of modification are most typically generated for modifying VLP expression and assembly. These modifications are designed to alter the interior, exterior or the interface between adjacent subunits in the assembled protein cage. To accomplish this, mutagenic primers can be used to: (i) alter the interior surface charge of the viral nucleic acid binding region by replacing basic residues (e.g. K, R) in the N terminus with acidic glutamic acids (Douglas et al., 2002b); (ii) delete interior residues from the N terminus (in CCMV, usually residues 4-37); (iii) insert a cDNA encoding an 11 amino acid peptide cell-targeting sequence (Graf et al., 1987) into a surface exposed loop and (iv) modify interactions between viral subunits by altering the metal binding sites (in CCMV, residues 81/148 mutant).

Recombinant Peptides

Size

In one embodiment, the peptides operably linked to a viral capsid sequence contain at least two amino acids. In another embodiment, the peptides are at least three, at least four, at least five, or at least six amino acids in length. In a separate embodiment, the peptides are at least seven amino acids long. The peptides can also be at least eight, at least nine, at least ten, at least 11, 12, 13, 14, 15, 16, 17, 18, 19, 20, 30, 45, 50, 60, 65, 75, 85, 95, 96, 99 or more amino acids long. In one embodiment, the peptides encoded are at least 25 kD.

In one embodiment, the peptide will contain from 2 to about 300 amino acids, or about 5 to about 250 amino acids, or about 5 to about 200 amino acids, or about 5 to about 150 amino acids, or about 5 to about 100 amino acids. In another embodiment, the peptide contains or about 10 to about 140 amino acids, or about 10 to about 120 amino acids, or about 10 to about 100 amino acids.

In one embodiment, the peptides or proteins operably linked to a viral capsid sequence will contain about 500 amino acids. In one embodiment, the peptide will contain less than 500 amino acids. In another embodiment, the peptide will contain up to about 300 amino acids, or up to about 250, or up to about 200, or up to about 180, or up to about 160, or up to about 150, or up to about 140, or up to about 120, or up to about 110, or up to about 100, or up to about 90, or up to about 80, or up to about 70, or up to about 60, or up to about 50, or up to about 40 or up to about 30 amino acids.

In one embodiment, the recombinant peptide fused to the icosahedral capsid is at least 7, at least 8, at least, 9, at least 10, at least 12, at least 15, at least 20, at least 25, at least 30, at least 35, at least 40, at least 45, at least 50, at least 55, at least 60, at least 65, at least 75, at least 85, at least 95, at least 99, or at least 100 amino acids.

In one embodiment of the present invention, the recombinant peptide contains at least one monomer of a desired target peptide. In an alternative embodiment, the recombinant peptide contains more than one monomer of a desired target peptide. In certain embodiments, the peptide is composed of at least two, at least 5, at least 10, at least 15 or at least 20 separate monomers that are operably linked as a concatameric peptide to the capsid. In another embodiment, the individual monomers in the concatameric peptide are linked by cleavable linker regions. In still another embodiment, the recombinant peptide is inserted into at least one surface loop of the icosahedral virus-like particle. In one embodiment, at least one monomer is inserted in a surface loop of the virus-like particle.

Classification

The peptides of interest that are fused to the viral capsids can be a heterologous protein that is not derived from the virus and, optionally, that is not derived from the same species as the cell.

The peptides of interest that are fused to the viral capsids can be functional peptides; structural peptides; antigenic peptides, toxic peptides, antimicrobial peptides, fragments thereof; precursors thereof; combinations of any of the foregoing; and/or concatamers of any of the foregoing. In one embodiment of the present invention, the recombinant peptide is a therapeutic peptide useful for human and animal treatments.

Functional peptides include, but are not limited to, e.g.: bio-active peptides (i.e. peptides that exert, elicit, or otherwise result in the initiation, enhancement, prolongation, attenuation, termination, or prevention of a biological function or activity in or of a biological entity, e.g., an organism, cell, culture, tissue, organ, or organelle); catalytic peptides; microstructure- and nanostructure-active peptides (i.e. peptides that form part of engineered micro- or nano-structures in which, or in conjunction with which, they perform an activity, e.g., motion, energy transduction); and stimulant peptides (e.g., peptide flavorings, colorants, odorants, pheromones, attractants, deterrents, and repellants).

Bio-active peptides include, but are not limited to, e.g.: immunoactive peptides (e.g., antigenic peptides, allergenic peptides, peptide immunoregulators, peptide immunomodulators); signaling and signal transduction peptides (e.g., peptide hormones, cytokines, and neurotransmitters; receptors; agonist and antagonist peptides; peptide targeting and secretion signal peptides); and bio-inhibitory peptides (e.g., toxic, biocidal, or biostatic peptides, such as peptide toxins and antimicrobial peptides).

Structural peptides include, but are not limited to, e.g.: peptide aptamers; folding peptides (e.g., peptides promoting or inducing formation or retention of a physical conformation in another molecule); adhesion-promoting peptides (e.g., adhesive peptides, cell-adhesion-promoting peptides); interfacial peptides (e.g., peptide surfactants and emulsifiers); microstructure and nanostructure-architectural peptides (i.e. structural peptides that form part of engineered micro- or nano-structures); and pre-activation peptides (e.g., leader peptides of pre-, pro-, and pre-pro-proteins and -peptides; inteins).

Catalytic Peptides include, e.g., apo B RNA-editing cytidine deaminase peptides; catalytic peptides of glutaminyl-tRNA synthetases; catalytic peptides of aspartate transcarbamoylases; plant Type 1 ribosome-inactivating peptides; viral catalytic peptides such as, e.g., the foot-and-mouth disease virus [FMDV-2A] catalytic peptide; matrix metalloproteinase peptides; and catalytic metallo-oligopeptides.

The peptide can also be a peptide epitopes, haptens, or a related peptides (e.g., antigenic viral peptides; virus related peptides, e.g., HIV-related peptides, hepatitis-related peptides; antibody idiotypic domains; cell surface peptides; antigenic human, animal, protist, plant, fungal, bacterial, and/or archaeal peptides; allergenic peptides and allergen desensitizing peptides).

The peptide can also be a peptide immunoregulators or immunomodulators (e.g., interferons, interleukins, peptide immunodepressants and immunopotentiators); an antibody peptides (e.g., single chain antibodies; single chain antibody fragments and constructs, e.g., single chain Fv molecules; antibody light chain molecules, antibody heavy chain molecules, domain-deleted antibody light or heavy chain molecules; single chain antibody domains and molecules, e.g., a CH1, CH1-3, CH3, CH1-4, CH4, VHCH1, CL, CDR1, or FR1-CDR1-FR2 domain; paratopic peptides; microantibodies); another binding peptide (e.g., peptide aptamers, intracellular and cell surface receptor proteins, receptor fragments; anti-tumor necrosis factor peptides).

The peptide can also be an enzyme substrate peptide or an enzyme inhibitor peptide (e.g., caspase substrates and inhibitors, protein kinase substrates and inhibitors, fluorescence-resonance-energy transfer-peptide enzyme substrates).

The peptide can also be a cell surface receptor peptide ligand, agonist, and antagonist (e.g., caeruleins, dynorphins, orexins, pituitary adenylate cyclase activating peptides, tumor necrosis factor peptides; synthetic peptide ligands, agonists, and antagonists); a peptide hormone (e.g., endocrine, paracrine, and autocrine hormones, including, e.g.: amylins, angiotensins, bradykinins, calcitonins, cardioexcitatory neuropeptides, casomorphins, cholecystokinins, corticotropins and corticotropin-related peptides, differentiation factors, endorphins, endothelins, enkephalins, erythropoietins, exendins, follicle-stimulating hormones, galanins, gastrins, glucagons and glucagon-like peptides, gonadotropins, growth hormones and growth factors, insulins, kallidins, kinins, leptins, lipotropic hormones, luteinizing hormones, melanocyte stimulating hormones, melatonins, natriuretic peptides, neurokinins, neuromedins, nociceptins, osteocalcins, oxytocins (i.e. ocytocins), parathyroid hormones, pleiotrophins, prolactins, relaxins, secretins, serotonins, sleep-inducing peptides, somatomedins, thymopoietins, thyroid stimulating hormones, thyrotropins, urotensins, vasoactive intestinal peptides, vasopressins); a peptide cytokine, chemokine, virokine, and viroceptor hormone releasing and release-inhibiting peptide (e.g., corticotropin-releasing hormones, cortistatins, follicle-stimulating-hormone-releasing factors, gastric inhibitory peptides, gastrin releasing peptides, gonadotropin-releasing hormones, growth hormone releasing hormones, luteinizing hormone-releasing hormones, melanotropin-releasing hormones, melanotropin-release inhibiting factors; nocistatins, pancreastatins, prolactinreleasing peptides, prolactin release-inhibiting factors; somatostatins; thyrotropin releasing hormones); a peptide neurotransmitter or channel blocker (e.g., bombesins, neuropeptide Y, neurotensins, substance P) a peptide toxin, toxin precursor peptide, or toxin peptide portion. In certain embodiments, a peptide toxin contains no D-amino acids. Toxin precursor peptides can be those that contain no D-amino acids and/or that have not been converted by posttranslational modification into a native toxin structure, such as, e.g., by action of a D configuration inducing agent (e.g., a peptide isomerase(s) or epimeras(e) or racemase(s) or transaminase(s)) that is capable of introducing a D-configuration in an amino acid(s), and/or by action of a cyclizing agent (e.g., a peptide thioesterase, or a peptide ligase such as a trans-splicing protein or intein) that is capable of form a cyclic peptide structure.

Toxin peptide portions can be the linear or pre-cyclized oligo- and poly-peptide portions of peptide-containing toxins. Examples of peptide toxins include, e.g., agatoxins, amatoxins, charybdotoxins, chlorotoxins, conotoxins, dendrotoxins, insectotoxins, margatoxins, mast cell degranulating peptides, saporins, sarafotoxins; and bacterial exotoxins such as, e.g., anthrax toxins, botulism toxins, diphtheria toxins, and tetanus toxins.

The peptide can also be a metabolism- and digestion-related peptide (e.g., cholecystokinin-pancreozymin peptides, peptide yy, pancreatic peptides, motilins); a cell adhesion modulating or mediating peptide, extracellular matrix peptide (e.g., adhesins, selectins, laminins); a neuroprotectant or myelination-promoting peptide; an aggregation inhibitory peptide (e.g., cell or platelet aggregation inhibitor peptides, amyloid formation or deposition inhibitor peptides); a joining peptide (e.g., cardiovascular joining neuropeptides, iga joining peptides); or a miscellaneous peptide (e.g., agouti-related peptides, amyloid peptides, bone-related peptides, cell-permeable peptides, conantokins, contryphans, contulakins, myelin basic protein, and others).

In certain embodiments, the peptide of interest is exogenous to the selected viral capsid. Peptides may be either native or synthetic in sequence (and their coding sequences may be either native or synthetic nucleotide sequences). Thus, e.g., native, modified native, and entirely artificial sequences of amino acids are encompassed. The sequences of the nucleic acid molecules encoding these amino acid sequences likewise may be native, modified native, or entirely artificial nucleic acid sequences, and may be the result of, e.g., one or more rational or random mutation and/or recombination and/or synthesis and/or selection process employed (i.e. applied by human agency) to obtain the nucleic acid molecules.

The coding sequence can be a native coding sequence for the target peptide, if available, but will more typically be a coding sequence that has been selected, improved, or optimized for use in the selected expression host cell: for example, by synthesizing the gene to reflect the codon use preference of a host species. In one embodiment of the invention, the host species is a P. fluorescens, and the codon preference of P. fluorescens is taken into account when designing both the signal sequence and the peptide sequence.

Antigenic Peptides (Peptide Epitopes)

In one embodiment, an antigenic peptide is produced through expression with a viral capsid. The antigenic peptide can be selected from those that are antigenic peptides of human or animal pathogenic agents, including infectious agents, parasites, cancer cells, and other pathogenic agents. Such pathogenic agents also include the virulence factors and pathogenesis factors, e.g., exotoxins, endotoxins, et al., of those agents. The pathogenic agents may exhibit any level of virulence, i.e. they may be, e.g., virulent, avirulent, pseudo-virulent, semi-virulent, and so forth. In one embodiment, the antigenic peptide will contain an epitopic amino acid sequence from the pathogenic agent(s). In one embodiment, the epitopic amino acid sequence will include that of at least a portion of a surface peptide of at least one such agent. In one embodiment, the capsid-recombinant peptide virus like particles can be used as a vaccine in a human or animal application.

More than one antigenic peptide may be selected, in which case the resulting virus-like particles can present multiple different antigenic peptides. In a particularly embodiment of a multiple antigenic peptide format, the various antigenic peptides will all be selected from a plurality of epitopes from the same pathogenic agent. In a particular embodiment of a multi-antigenic-peptide format, the various antigenic peptides selected will all be selected from a plurality of closely related pathogenic agents, for example, different strains, subspecies, biovars, pathovars, serovars, or genovars of the same species or different species of the same genus.

In one embodiment, the pathogenic agent(s) will belong to at least one of the following groups: Bacteria and Mycoplasma agents including, but not limited to, pathogenic: Bacillus spp., e.g., Bacillus anthracis; Bartonella spp., e.g., B. quintana; Brucella spp.; Burkholderia spp., e.g., B. pseudomallei; Campylobacter spp.; Clostridium spp., e.g., C. tetani, C. botulinum; Coxiella spp., e.g., C. burnetii; Edwardsiella spp., e.g., E. tarda; Enterobacter spp., e.g., E. cloacae; Enterococcus spp., e.g., E. faecalis, E. faecium; Escherichia spp., e.g., E. coli; Francisella spp., e.g., F. tularensis; Haemophilus spp., e.g., H. influenzae; Klebsiella spp., e.g., K. pneumoniae; Legionella spp.; Listeria spp., e.g., L. monocytogenes; Meningococci and Gonococci, e.g., Neisseria spp.; Moraxella spp.; Mycobacterium spp., e.g., M. leprae, M. tuberculosis; Pneumococci, e.g., Diplococcus pneumoniae; Pseudomonas spp., e.g., P. aeruginosa; Rickettsia spp., e.g., R. prowazekii, R. rickettsii, R. typhi; Salmonella spp., e.g., S. typhi; Staphylococcus spp., e.g., S. aureus; Streptococcus spp., including Group A Streptococci and hemolytic Streptococci, e.g., S. pneumoniae, S. pyogenes; Streptomyces spp.; Shigella spp.; Vibrio spp., e.g., V. cholerae; and Yersinia spp., e.g., Y. pestis, Y. enterocolitica. Fungus and Yeast agents including, but not limited to, pathogenic: Alternaria spp.; Aspergillus spp.; Blastomyces spp., e.g., B. dermatiditis; Candida spp., e.g., C. albicans; Cladosporium spp.; Coccidiodes spp., e.g., C. immitis; Cryptococcus spp., e.g., C. neoformans; Histoplasma spp., e.g., H. capsulatum; and Sporothrix spp., e.g., S. schenckii.

In one embodiment, the pathogenic agent(s) will be from a protist agent including, but not limited to, pathogenic: Amoebae, including Acanthamoeba spp., Amoeba spp., Naegleria spp., Entamoeba spp., e.g., E. histolytica; Cryptosporidium spp., e.g., C. parvum; Cyclospora spp.; Encephalitozoon spp., e.g., E. intestinalis; Enterocytozoon spp.; Giardia spp., e.g., G. lamblia; Isospora spp.; Microsporidium spp.; Plasmodium spp., e.g., P. falciparum, P. malariae, P. ovale, P. vivax; Toxoplasma spp., e.g., T. gondii; and Trypanosoma spp., e.g., T. brucei.

In one embodiment, the pathogenic agent(s) will be from a parasitic agent (e.g., helminthic parasites) including, but not limited to, pathogenic: Ascaris spp., e.g., A. lumbricoides; Dracunculus spp., e.g., D. medinensis; Onchocerca spp., e.g., O. volvulus; Schistosoma spp.; Trichinella spp., e.g., T. spiralis; and Trichuris spp., e.g., T. trichiura.

In another embodiment, the pathogenic agent(s) will be from a viral agent including, but not limited to, pathogenic: Adenoviruses; Arenaviruses, e.g., Lassa Fever viruses; Astroviruses; Bunyaviruses, e.g., Hantaviruses, Rift Valley Fever viruses; Coronaviruses, Deltaviruses; Cytomegaloviruses, Epstein-Barr viruses, Herpes viruses, Varicella viruses; Filoviruses, e.g., Ebola viruses, Marburg viruses; Flaviruses, e.g., Dengue viruses, West Nile Fever viruses, Yellow Fever viruses; Hepatitis viruses; Influenzaviruses; Lentiviruses, T-Cell Lymphotropic viruses, other leukemia viruses; Norwalk viruses; Papillomaviruses, other tumor viruses; Paramyxoviruses, e.g., Measles viruses, Mumps viruses, Parainfluenzaviruses, Pneumoviruses, Sendai viruses; Parvoviruses; Picornaviruses, e.g., Cardioviruses, Coxsackie viruses, Echoviruses, Poliomyelitis viruses, Rhinoviruses, Other Enteroviruses; Poxviruses, e.g., Variola viruses, Vaccinia viruses, Parapoxviruses; Reoviruses, e.g., Coltiviruses, Orbiviruses, Rotaviruses; Rhabdoviruses, e.g., Lyssaviruses, Vesicular Stomatitis viruses; and Togaviruses, e.g., Rubella viruses, Sindbis viruses, Western Encephalitis viruses.

In one particular embodiment, the antigenic peptide is selected from the group consisting of a Canine parvovirus peptide, Bacillus anthracis protective antigen (PA) antigenic peptide, and an Eastern Equine Encephalitis virus antigenic peptide. In a particular embodiment, the antigenic peptide is the canine parvovirus-derived peptide with the amino acid sequence of SEQ. ID. NO: 7. In another particular embodiment, the antigenic peptide is the Bacillus anthracis protective antigen (PA) antigenic peptide with any one of the amino acid sequence of SEQ. ID. NOs: 9, 11, 13 or 15. In still another particular embodiment, the antigenic peptide is an Eastern equine Encephalitis virus antigenic peptide with the amino acid sequence of one of SEQ. ID. NOs:25 or 27.

Host-Cell Toxic Peptide

In another particular embodiment, the recombinant peptide is a peptide that is toxic to the host cell when in free monomeric form. In a more particular embodiment, the toxic peptide is an antimicrobial peptide.

In certain embodiments, the peptide of interest expressed in conjunction with a viral capsid will be a host cell toxic peptide. In certain embodiments, this protein will be an antimicrobial peptide. A host cell toxic peptide indicates a bio-inhibitory peptide that is biostatic, biocidal, or toxic to the host cell in which it is expressed, or to other cells in the cell culture or organism of which the host cell is a member, or to cells of the organism or species providing the host cells. In one embodiment, the host-cell-toxic peptide will be a bioinhibitory peptide that is biostatic, biocidal, or toxic to the host cell in which it is expressed. Some examples of host-cell-toxic peptides include, but are not limited to: peptide toxins, anti-microbial peptides, and other antibiotic peptides.

Anti-Microbial Peptides include, e.g., anti-bacterial peptides such as, e.g., magainins, betadefensins, some alpha-defensins; cathelicidins; histatins; anti-fungal peptides; antiprotozoal peptides; synthetic AMPs; peptide antibiotics or the linear or pre-cyclized oligo- or poly-peptide portions thereof; other antibiotic peptides (e.g., antheimintic peptides, hemolytic peptides, tumoricidal peptides); and anti-viral peptides (e.g., some alpha-defensins; virucidal peptides; peptides that inhibit viral infection). In one particular embodiment, the antimicrobial peptide is the D2A21 peptide with the amino acid sequence of SEQ ID NO:20. In another embodiment, the antimicrobial peptide is antimicrobial peptide PBF20 with the amino acid sequence corresponding substantially to SEQ ID NO:24.

Cells for Use in Expressing the VLP

The cell used as a host for the expression of the viral capsid or viral capsid fusion peptide (also referred to as “host cell”) of the invention will be one in which the viral capsid does not allow replication or infection of the cell. In one embodiment, the viral capsid will be derived from a virus that does not infect the species of cell that the host cell is derived from. For example, in one embodiment, the viral capsid is derived from an icosahedral plant virus and is expressed in a host cell of a bacterial species. In another embodiment, the viral species infects mammals and the expression system includes a bacterial host cell.

In one embodiment, the host cell can be a prokaryote such as a bacterial cell including, but not limited to a Pseudomonas species. Typical bacterial cells are described, for example, in “Biological Diversity: Bacteria and Archaeans”, a chapter of the On-Line Biology Book, provided by Dr M J Farabee of the Estrella Mountain Community College, Arizona, USA at URL: http://www.emc.maricopa.edu/faculty/farabee/BIOBK/BioBookDiversity_{—}2.html. In certain embodiments, the host cell can be a Pseudomonad cell, and can typically be a P. fluorescens cell.

In one embodiment, the host cell can be a member of any species of eubacteria. The host can be a member any one of the taxa: Acidobacteria, Actinobacteira, Aquificae, Bacteroidetes, Chlorobi, Chlamydiae, Choroflexi, Chrysiogenetes, Cyanobacteria, Deferribacteres, Deinococcus, Dictyoglomi, Fibrobacteres, Firmicutes, Fusobacteria, Gemmatimonadetes, Lentisphaerae, Nitrospirae, Planctomycetes, Proteobacteria, Spirochaetes, Thermodesulfobacteria, Thermomicrobia, Thermotogae, Thermus (Thermales), or Verrucomicrobia. In an embodiment of a eubacterial host cell, the cell can be a member of any species of eubacteria, excluding Cyanobacteria.

The bacterial host can also be a member of any species of Proteobacteria. A proteobacterial host cell can be a member of any one of the taxa Alphaproteobacteria, Betaproteobacteria, Gammaproteobacteria, Deltaproteobacteria, or Epsilonproteobacteria. In addition, the host can be a member of any one of the taxa Alphaproteobacteria, Betaproteobacteria, or Gammaproteobacteria, and a member of any species of Gammaproteobacteria.

In one embodiment of a Gamma Proteobacterial host, the host will be member of any one of the taxa Aeromonadales, Alteromonadales, Enterobacteriales, Pseudomonadales, or Xanthomonadales; or a member of any species of the Enterobacteriales or Pseudomonadales. In one embodiment, the host cell can be of the order Enterobacteriales, the host cell will be a member of the family Enterobacteriaceae, or a member of any one of the genera Erwinia, Escherichia, or Serratia; or a member of the genus Escherichia. In one embodiment of a host cell of the order Pseudomonadales, the host cell will be a member of the family Pseudomonadaceae, even of the genus Pseudomonas. Gamma Proteobacterial hosts include members of the species Escherichia coli and members of the species Pseudomonas fluorescens.

Other Pseudomonas organisms may also be used. Pseudomonads and closely related species include Gram(−) Proteobacteria Subgroup 1, which include the group of Proteobacteria belonging to the families and/or genera described as “Gram-Negative Aerobic Rods and Cocci” by R. E. Buchanan and N. E. Gibbons (eds.), Bergey's Manual of Determinative Bacteriology, pp. 217-289 (8th ed., 1974) (The Williams & Wilkins Co., Baltimore, Md., USA) (hereinafter “Bergey (1974)”). Table 1 presents these families and genera of organisms.

TABLE 1
FAMILIES AND GENERA LISTED IN THE PART, “GRAM-
NEGATIVE AEROBIC RODS AND COCCI” (IN BERGEY (1974))

| | |
|---|---|
| Family I. Pseudomonadaceae | Gluconobacter |
| | Pseudomonas |
| | Xanthomonas |
| | Zoogloea |
| Family II. Azotobacteraceae | Azomonas |
| | Azotobacter |
| | Beijerinckia |
| | Derxia |
| Family III. Rhizobiaceae | Agrobacterium |
| | Rhizobium |
| Family IV. Methylomonadaceae | Methylococcus |
| | Methylomonas |
| Family V. Halobacteriaceae | Halobacterium |
| | Halococcus |
| Other Genera | Acetobacter |
| | Alcaligenes |
| | Bordetella |
| | Brucella |
| | Francisella |
| | Thermus |

“Gram(−) Proteobacteria Subgroup 1” also includes Proteobacteria that would be classified in this heading according to the criteria used in the classification. The heading also includes groups that were previously classified in this section but are no longer, such as the genera Acidovorax, Brevundimonas, Burkholderia, Hydrogenophaga, Oceanimonas, Ralstonia, and Stenotrophomonas, the genus Sphingomonas (and the genus Blastomonas, derived therefrom), which was created by regrouping organisms belonging to (and previously called species of) the genus Xanthomonas, the genus Acidomonas, which was created by regrouping organisms belonging to the genus Acetobacter as defined in Bergey (1974). In addition hosts can include cells from the genus Pseudomonas, Pseudomonas enalia (ATCC 14393), Pseudomonas nigrifaciens (ATCC 19375), and Pseudomonas putrefaciens (ATCC 8071), which have been reclassified respectively as Alteromonas haloplanktis, Alteromonas nigrifaciens, and Alteromonas putrefaciens. Similarly, e.g., Pseudomonas acidovorans (ATCC 15668) and Pseudomonas testosteroni (ATCC 11996) have since been reclassified as Comamonas acidovorans and Comamonas testosteroni, respectively; and Pseudomonas nigrifaciens (ATCC 19375) and Pseudomonas piscicida (ATCC 15057) have been reclassified respectively as Pseudoalteromonas nigrifaciens and Pseudoalteromonas piscicida. “Gram(−) Proteobacteria Subgroup 1” also includes Proteobacteria classified as belonging to any of the families: Pseudomonadaceae, Azotobacteraceae (now often called by the synonym, the “Azotobacter group” of Pseudomonadaceae), Rhizobiaceae, and Methylomonadaceae (now often called by the synonym, “Methylococcaceae”). Consequently, in addition to those genera otherwise described herein, further Proteobacterial genera falling within “Gram(−) Proteobacteria Subgroup 1” include: 1) Azotobacter group bacteria of the genus Azorhizophilus; 2) Pseudomonadaceae family bacteria of the genera Cellvibrio, Oligella, and Teredinibacter; 3) Rhizobiaceae family bacteria of the genera Chelatobacter, Ensifer, Liberibacter (also called “Candidatus Liberibacter”), and Sinorhizobium; and 4) Methylococcaceae family bacteria of the genera Methylobacter, Methylocaldum, Methylomicrobium, Methylosarcina, and Methylosphaera.

In another embodiment, the host cell is selected from “Gram(−) Proteobacteria Subgroup 2.” “Gram(−) Proteobacteria Subgroup 2” is defined as the group of Proteobacteria of the following genera (with the total numbers of catalog-listed, publicly-available, deposited strains thereof indicated in parenthesis, all deposited at ATCC, except as otherwise indicated): Acidomonas (2); Acetobacter (93); Gluconobacter (37); Brevundimonas (23); Beijerinckia (13); Derxia (2); Brucella (4); Agrobacterium (79); Chelatobacter (2); Ensifer (3); Rhizobium (144); Sinorhizobium (24); Blastomonas (1); Sphingomonas (27); Alcaligenes (88); Bordetella (43); Burkholderia (73); Ralstonia (33); Acidovorax (20); Hydrogenophaga (9); Zoogloea (9); Methylobacter (2); Methylocaldum (1 at NCIMB); Methylococcus (2); Methylomicrobium (2); Methylomonas (9); Methylosarcina (1); Methylosphaera; Azomonas (9); Azorhizophilus (5); Azotobacter (64); Cellvibrio (3); Oligella (5); Pseudomonas (1139); Francisella (4); Xanthomonas (229); Stenotrophomonas (50); and Oceanimonas (4).

Exemplary host cell species of “Gram(−) Proteobacteria Subgroup 2” include, but are not limited to the following bacteria (with the ATCC or other deposit numbers of exemplary strain(s) thereof shown in parenthesis): Acidomonas methanolica (ATCC 43581); Acetobacter aceti (ATCC 15973); Gluconobacter oxydans (ATCC 19357); Brevundimonas diminuta (ATCC 11568); Beijerinckia indica (ATCC 9039 and ATCC 19361); Derxia gummosa (ATCC 15994); Brucella melitensis (ATCC 23456), Brucella abortus (ATCC 23448); Agrobacterium tumefaciens (ATCC 23308), Agrobacterium radiobacter (ATCC 19358), Agrobacterium rhizogenes (ATCC 11325); Chelatobacter heintzii (ATCC 29600); Ensifer adhaerens (ATCC 33212); Rhizobium leguminosarum (ATCC 10004); Sinorhizobium fredii (ATCC 35423); Blastomonas natatoria (ATCC 35951); Sphingomonas paucimobilis (ATCC 29837); Alcaligenes faecalis (ATCC 8750); Bordetella pertussis (ATCC 9797); Burkholderia cepacia (ATCC 25416); Ralstonia pickettii (ATCC 27511); Acidovorax facilis (ATCC 11228); Hydrogenophaga flava (ATCC 33667); Zoogloea ramigera (ATCC 19544); Methylobacter luteus (ATCC 49878); Methylocaldum gracile (NCIMB 11912); Methylococcus capsulatus (ATCC 19069); Methylomicrobium agile (ATCC 35068); Methylomonas methanica (ATCC 35067); Methylosarcina fibrata (ATCC 700909); Methylosphaera hansonii (ACAM 549); Azomonas agilis (ATCC 7494); Azorhizophilus paspali (ATCC 23833); Azotobacter chroococcum (ATCC 9043); Cellvibrio mixtus (UQM 2601); Oligella urethralis (ATCC 17960); Pseudomonas aeruginosa (ATCC 10145), Pseudomonas fluorescens (ATCC 35858); Francisella tularensis (ATCC 6223); Stenotrophomonas maltophilia (ATCC 13637); Xanthomonas campestris (ATCC 33913); and Oceanimonas doudoroffii (ATCC 27123).

In another embodiment, the host cell is selected from “Gram(−) Proteobacteria Subgroup 3.” “Gram(−) Proteobacteria Subgroup 3” is defined as the group of Proteobacteria of the following genera: Brevundimonas; Agrobacterium; Rhizobium; Sinorhizobium; Blastomonas; Sphingomonas; Alcaligenes; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

In another embodiment, the host cell is selected from “Gram(−) Proteobacteria Subgroup 4.” “Gram(−) Proteobacteria Subgroup 4” is defined as the group of Proteobacteria of the following genera: Brevundimonas; Blastomonas; Sphingomonas; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

In an embodiment, the host cell is selected from “Gram(−) Proteobacteria Subgroup 5.” “Gram(−) Proteobacteria Subgroup 5” is defined as the group of Proteobacteria of the following genera: Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

The host cell can be selected from “Gram(−) Proteobacteria Subgroup 6.” “Gram(−) Proteobacteria Subgroup 6” is defined as the group of Proteobacteria of the following genera: Brevundimonas; Blastomonas; Sphingomonas; Burkholderia; Ralstonia; Acidovorax; Hydrogenophaga; Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram(−) Proteobacteria Subgroup 7.” “Gram(−) Proteobacteria Subgroup 7” is defined as the group of Proteobacteria of the following genera: Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram(−) Proteobacteria Subgroup 8.” “Gram(−) Proteobacteria Subgroup 8” is defined as the group of Proteobacteria of the following genera: Brevundimonas; Blastomonas; Sphingomonas; Burkholderia; Ralstonia; Acidovorax; Hydrogenophaga; Pseudomonas; Stenotrophomonas; Xanthomonas; and Oceanimonas.

The host cell can be selected from “Gram(−) Proteobacteria Subgroup 9.” “Gram(−) Proteobacteria Subgroup 9” is defined as the group of Proteobacteria of the following genera: Brevundimonas; Burkholderia; Ralstonia; Acidovorax; Hydrogenophaga; Pseudomonas; Stenotrophomonas; and Oceanimonas.

The host cell can be selected from “Gram(−) Proteobacteria Subgroup 10.” “Gram(−) Proteobacteria Subgroup 10” is defined as the group of Proteobacteria of the following genera: Burkholderia; Ralstonia; Pseudomonas; Stenotrophomonas; and Xanthomonas.

The host cell can be selected from “Gram(−) Proteobacteria Subgroup 11.” “Gram(−) Proteobacteria Subgroup 11” is defined as the group of Proteobacteria of the genera: Pseudomonas; Stenotrophomonas; and Xanthomonas. The host cell can be selected from “Gram(−) Proteobacteria Subgroup 12.” “Gram(−) Proteobacteria Subgroup 12” is defined as the group of Proteobacteria of the following genera: Burkholderia; Ralstonia; Pseudomonas. The host cell can be selected from “Gram(−) Proteobacteria Subgroup 13.” “Gram(−) Proteobacteria Subgroup 13” is defined as the group of Proteobacteria of the following genera: Burkholderia; Ralstonia; Pseudomonas; and Xanthomonas. The host cell can be selected from “Gram(−) Proteobacteria Subgroup 14.” “Gram(−) Proteobacteria Subgroup 14” is defined as the group of Proteobacteria of the following genera: Pseudomonas and Xanthomonas. The host cell can be selected from “Gram(−) Proteobacteria Subgroup 15.” “Gram(−) Proteobacteria Subgroup 15” is defined as the group of Proteobacteria of the genus Pseudomonas.

The host cell can be selected from “Gram(−) Proteobacteria Subgroup 16.” “Gram(−) Proteobacteria Subgroup 16” is defined as the group of Proteobacteria of the following Pseudomonas species (with the ATCC or other deposit numbers of exemplary strain(s) shown in parenthesis): Pseudomonas abietaniphila (ATCC 700689); Pseudomonas aeruginosa (ATCC 10145); Pseudomonas alcaligenes (ATCC 14909); Pseudomonas anguilliseptica (ATCC 33660); Pseudomonas citronellolis (ATCC 13674); Pseudomonas flavescens (ATCC 51555); Pseudomonas mendocina (ATCC 25411); Pseudomonas nitroreducens (ATCC 33634); Pseudomonas oleovorans (ATCC 8062); Pseudomonas pseudoalcaligenes (ATCC 17440); Pseudomonas resinovorans (ATCC 14235); Pseudomonas straminea (ATCC 33636); Pseudomonas agarici (ATCC 25941); Pseudomonas alcaliphila; Pseudomonas alginovora; Pseudomonas andersonii; Pseudomonas asplenii (ATCC 23835); Pseudomonas azelaica (ATCC 27162); Pseudomonas beijerinckii (ATCC 19372); Pseudomonas borealis; Pseudomonas boreopolis (ATCC 33662); Pseudomonas brassicacearum; Pseudomonas butanovora (ATCC 43655); Pseudomonas cellulosa (ATCC 55703); Pseudomonas aurantiaca (ATCC 33663); Pseudomonas chlororaphis (ATCC 9446, ATCC 13985, ATCC 17418, ATCC 17461); Pseudomonas fragi (ATCC 4973); Pseudomonas lundensis (ATCC 49968); Pseudomonas taetrolens (ATCC 4683); Pseudomonas cissicola (ATCC 33616); Pseudomonas coronafaciens; Pseudomonas diterpeniphila; Pseudomonas elongata (ATCC 10144); Pseudomonas flectens (ATCC 12775); Pseudomonas azotoformans; Pseudomonas brenneri; Pseudomonas cedrella; Pseudomonas corrugata (ATCC 29736); Pseudomonas extremorientalis; Pseudomonas fluorescens (ATCC 35858); Pseudomonas gessardii; Pseudomonas libanensis; Pseudomonas mandelii (ATCC 700871); Pseudomonas marginalis (ATCC 10844); Pseudomonas migulae; Pseudomonas mucidolens (ATCC 4685); Pseudomonas orientalis; Pseudomonas rhodesiae; Pseudomonas synxantha (ATCC 9890); Pseudomonas tolaasii (ATCC 33618); Pseudomonas veronii (ATCC 700474); Pseudomonas frederiksbergensis; Pseudomonas geniculata (ATCC 19374); Pseudomonas gingeri; Pseudomonas graminis; Pseudomonas grimontii; Pseudomonas halodenitrificans; Pseudomonas halophila; Pseudomonas hibiscicola (ATCC 19867); Pseudomonas huttiensis (ATCC 14670); Pseudomonas hydrogenovora; Pseudomonas jessenii (ATCC 700870); Pseudomonas kilonensis; Pseudomonas lanceolata (ATCC 14669); Pseudomonas lini; Pseudomonas marginata (ATCC 25417); Pseudomonas mephitica (ATCC 33665); Pseudomonas denitrificans (ATCC 19244); Pseudomonas pertucinogena (ATCC 190); Pseudomonas pictorum. (ATCC 23328); Pseudomonas psychrophila; Pseudomonas fulva (ATCC 31418); Pseudomonas monteilii (ATCC 700476); Pseudomonas mosselii; Pseudomonas oryzihabitans (ATCC 43272); Pseudomonas plecoglossicida (ATCC 700383); Pseudomonas putida (ATCC 12633); Pseudomonas reactans; Pseudomonas spinosa (ATCC 14606); Pseudomonas balearica; Pseudomonas luteola (ATCC 43273); Pseudomonas stutzeri (ATCC 17588); Pseudomonas amygdali (ATCC 33614); Pseudomonas avellanae (ATCC 700331); Pseudomonas caricapapayae (ATCC 33615); Pseudomonas cichorii (ATCC 10857); Pseudomonas ficuserectae (ATCC 35104); Pseudomonas fuscovaginae; Pseudomonas meliae (ATCC 33050); Pseudomonas syringae (ATCC 19310); Pseudomonas viridiflava (ATCC 13223); Pseudomonas thermocarboxydovorans (ATCC 35961); Pseudomonas thermotolerans; Pseudomonas thivervalensis; Pseudomonas vancouverensis (ATCC 700688); Pseudomonas wisconsinensis; and Pseudomonas xiamenensis.

The host cell can be selected from “Gram(−) Proteobacteria Subgroup 17.” “Gram(−) Proteobacteria Subgroup 17” is defined as the group of Proteobacteria known in the art as the “fluorescent Pseudomonads” including those belonging, e.g., to the following Pseudomonas species: Pseudomonas azotoformans; Pseudomonas brenneri; Pseudomonas cedrella; Pseudomonas corrugata; Pseudomonas extremorientalis; Pseudomonas fluorescens; Pseudomonas gessardii; Pseudomonas libanensis; Pseudomonas mandelii; Pseudomonas marginalis; Pseudomonas migulae; Pseudomonas mucidolens; Pseudomonas orientalis; Pseudomonas rhodesiae; Pseudomonas synxantha; Pseudomonas tolaasii; and Pseudomonas veronii.

In this embodiment, the host cell can be selected from “Gram(−) Proteobacteria Subgroup 18.” “Gram(−) Proteobacteria Subgroup 18” is defined as the group of all subspecies, varieties, strains, and other sub-special units of the species Pseudomonas fluorescens, including those belonging, e.g., to the following (with the ATCC or other deposit numbers of exemplary strain(s) shown in parenthesis): Pseudomonas fluorescens biotype A, also called biovar 1 or biovar I (ATCC 13525); Pseudomonas fluorescens biotype B, also called biovar 2 or biovar II (ATCC 17816); Pseudomonas fluorescens biotype C, also called biovar 3 or biovar III (ATCC 17400); Pseudomonas fluorescens biotype F, also called biovar 4 or biovar IV (ATCC 12983); Pseudomonas fluorescens biotype G, also called biovar 5 or biovar V (ATCC 17518); Pseudomonas fluorescens biovar VI; Pseudomonas fluorescens Pf0-1; Pseudomonas fluorescens Pf-5 (ATCC BAA-477); Pseudomonas fluorescens SBW25; and Pseudomonas fluorescens subsp. cellulosa (NCIMB 10462).

The host cell can be selected from “Gram(−) Proteobacteria Subgroup 19.” “Gram(−) Proteobacteria Subgroup 19” is defined as the group of all strains of Pseudomonas fluorescens biotype A. A particular strain of this biotype is P. fluorescens strain MB101 (see U.S. Pat. No. 5,169,760 to Wilcox), and derivatives thereof. An example of a derivative thereof is P. fluorescens strain MB214, constructed by inserting into the MB101 chromosomal asd (aspartate dehydrogenase gene) locus, a native E. coli PlacI-lacI-lacZYA construct (i.e. in which PlacZ was deleted).

Additional P. fluorescens strains that can be used in the present invention include Pseudomonas fluorescens Migula and Pseudomonas fluorescens Loitokitok, having the following ATCC designations: [NCIB 8286]; NRRL B-1244; NCIB 8865 strain CO1; NCIB 8866 strain CO₂; 1291 [ATCC 17458; IFO 15837; NCIB 8917; LA; NRRL B-1864; pyrrolidine; PW2 [ICMP 3966; NCPPB 967; NRRL B-899]; 13475; NCTC 10038; NRRL B-1603 [6; IFO 15840]; 52-1C; CCEB 488-A [BU 140]; CCEB 553 [IEM 15/47]; IAM 1008 [AHH-27]; LAM 1055 [AHH-23]; 1 [IFO 15842]; 12 [ATCC 25323; NIH 11; den Dooren de Jong 216]; 18 [IFO 15833; WRRL P-7]; 93 [TR-10]; 108 [52-22; IFO 15832]; 143 [IFO 15836; PL]; 149 [2-40-40; IFO 15838]; 182 [IFO 3081; PJ 73]; 184 [IFO 15830]; 185 [W2 L-1]; 186 [IFO 15829; PJ 79]; 187 [NCPPB 263]; 188 [NCPPB 316]; 189 [PJ227; 1208]; 191 [IFO 15834; PJ 236; 22/1]; 194 [Klinge R-60; PJ 253]; 196 [PJ 288]; 197 [PJ 290]; 198 [PJ 302]; 201 [PJ 368]; 202 [PJ 372]; 203 [PJ 376]; 204 [IFO 15835; PJ 682]; 205 [PJ 686]; 206 [PJ 692]; 207 [PJ 693]; 208 [PJ 722]; 212 [PJ 832]; 215 [PJ 849]; 216 [PJ 885]; 267 [B-9]; 271 [B-1612]; 401 [C71A; IFO 15831; PJ 187]; NRRL B-3178 [4; IFO 15841]; KY 8521; 3081; 30-21; [IFO 3081]; N; PYR; PW; D946-B83 [BU 2183; FERM-P 3328]; P-2563 [FERM-P 2894; IFO 13658]; LIM-1126 [43F]; M-1; A506 [A5-06]; A505 [A5-05-1]; A526 [A5-26]; B69; 72; NRRL B-4290; PMW6 [NCIB 11615]; SC 12936; A1 [IFO 15839]; F 1847 [CDC-EB]; F 1848 [CDC 93]; NCIB 10586; P17; F-12; AmMS 257; PRA25; 6133D02; 6519E01; N1; SC15208; BNL-WVC; NCTC 2583 [NCIB 8194]; H13; 1013 [ATCC 11251; CCEB 295]; IFO 3903; 1062; or Pf-5.

### II. Nucleic Acid Constructs

The present invention further provides nucleic acid constructs encoding a fusion peptide of an icosahedral capsid and a recombinant peptide. In one embodiment, a nucleic acid construct for use in transforming a Pseudomonad host cell including a) a nucleic acid encoding a recombinant peptide, and b) a nucleic acid sequence encoding an icosahedral capsid is provided, wherein the nucleic acid of a) and the nucleic acid of b) are operably linked to form a fusion protein when expressed in a cell.

In certain embodiments, the vector can include sequence for multiple capsids, or for multiple peptides of interest. In one embodiment, the vector can include at least two different capsid-peptide coding sequences. In one embodiment, the coding sequences are linked to the same promoter. In certain embodiments, the coding sequences are separated by an internal ribosomal binding site. In other embodiments, the coding sequences are linked by a linker sequence that allows the formation of virus like particles in the cell. In another embodiment, the coding sequences are linked to different promoters. These promoters may be driven by the same induction conditions. In another embodiment, multiple vectors encoding different capsid-peptide combinations are provided. The multiple vectors can include promoters that are driven by the same induction conditions, or by different induction conditions. In one embodiment, the promoter is a lac promoter, or a derivative of the lac promoter such as a tac promoter.

The coding sequence for a peptide of interest can be inserted into the coding sequence for a viral capsid or capsid in a predetermined site. The peptide can also be inserted at a non-predetermined site and cells screened for production of VLPs. In one embodiment, the peptide is inserted into the capsid coding sequence so as to be expressed as a loop during formation of a VLP. In one embodiment, one peptide coding sequence is included in the vector, however in other embodiments, multiple sequences are included. The multiple sequences can be in the form of concatamers, for example concatamers linked by cleavable linker sequences.

Peptides may be inserted at more than one insertion site in a capsid. Thus, peptides may be inserted in more than one surface loop motif of a capsid; peptides may also be inserted at multiple sites within a given loop motif. The individual functional and/or structural peptide(s) of the insert(s), and/or the entire peptide insert(s), may be separated by cleavage sites, i.e. sites at which an agent that cleaves or hydrolyzes protein can act to separate the peptide(s) from the remainder of the capsid structure or assemblage.

Peptides may be inserted within external-facing loop(s) and/or within internal-facing loop(s), i.e. within loops of the capsid that face respectively away from or toward the center of the capsid. Any amino acid or peptide bond in a surface loop of a capsid can serve as an insertion for the peptide. Typically, the insertion site will be selected at about the center of the loop, i.e. at about the position located most distal from the center of the tertiary structure of the folded capsid peptide. The peptide coding sequence may be operably inserted within the position of the capsid coding sequence corresponding to this approximate center of the selected loop(s). This includes the retention of the reading frame for that portion of the peptide sequence of the capsid that is synthesized downstream from the peptide insertion site.

In another embodiment, the peptide can be inserted at the amino terminus of the capsid. The peptide can be linked to the capsid through one or more linker sequences, including the cleavable linkers described above. In yet another embodiment, the peptide can be inserted at the carboxy terminus of the capsid. The peptide can also be linked to the carboxy terminus through one or more linkers, which can be cleavable by chemical or enzymatic hydrolysis. In one embodiment, peptide sequences are linked at both the amino and carboxy termini, or at one terminus and at at least one internal location, such as a location that is expressed on the surface of the capsid in its three dimensional conformation.

In one embodiment, the peptide can be inserted into the capsid from a Cowpea Chlorotic mosaic virus. In one particular embodiment, the peptide can be inserted at amino acid 129 of the CCMV virus. In another embodiment, the peptide sequence can be inserted at amino acids 60, 61, 62 or 63 of the CCMV virus. In still another embodiment, the peptide can be inserted at both amino acids 129 and amino acids 60-63 of the CCMV virus.

In a particular embodiment, the present invention provides a nucleic acid construct including a) a nucleic acid encoding an antimicrobial peptide, and b) a nucleic acid encoding an icosahedral capsid, wherein the nucleic acid of a) and the nucleic acid of b) are operably linked to form a fusion protein when expressed in a cell. Other capsids and recombinant peptides useful in constructing the nucleic acid construct are disclosed above.

Promoters

In one embodiment, the nucleic acid construct includes a promoter sequence operably attached to the nucleic acid sequence encoding the capsid-recombinant peptide fusion peptide. An operable attachment or linkage refers to any configuration in which the transcriptional and any translational regulatory elements are covalently attached to the described sequence so that by action of the host cell, the regulatory elements can direct the expression of the sequence of interest.

In a fermentation process, once expression of the target recombinant peptide is induced, it is ideal to have a high level of production in order to maximize efficiency of the expression system. The promoter initiates transcription and is generally positioned 10-100 nucleotides upstream of the ribosome binding site. Ideally, a promoter will be strong enough to allow for recombinant peptide accumulation of around 50% of the total cellular protein of the host cell, subject to tight regulation, and easily (and inexpensively) induced.

The promoters used in accordance with the present invention may be constitutive promoters or regulated promoters. Examples of commonly used inducible promoters and their subsequent inducers include lac (IPTG), lacUV5 (IPTG), tac (IPTG), trc (IPTG), P_{syn} (IPTG), trp (tryptophan starvation), araBAD (1-arabinose), lpp^{a} (IPTG), lpp-lac (IPTG), phoA (phosphate starvation), recA (nalidixic acid), proU (osmolarity), cst-1 (glucose starvation), tetA (tretracylin), cadA (pH), nar (anaerobic conditions), PL (thermal shift to 42° C.), cspA (thermal shift to 20° C.), T7 (thermal induction), T7-lac operator (IPTG), T3-lac operator (IPTG), T5-lac operator (IPTG), T4 gene32 (T4 infection), nprM-lac operator (IPTG), Pm (alkyl- or halo-benzoates), Pu (alkyl- or halo-toluenes), Psal (salicylates), and VHb (oxygen). See, for example, Makrides, S. C. (1996) Microbiol. Rev. 60, 512-538; Hannig G. & Makrides, S. C. (1998) TIBTECH 16, 54-60; Stevens, R. C. (2000) Structures 8, R177-R185. See, e.g.: J. Sanchez-Romero & V. De Lorenzo, Genetic Engineering of Nonpathogenic Pseudomonas strains as Biocatalysts for Industrial and Environmental Processes, in Manual of Industrial Microbiology and Biotechnology (A. Demain & J. Davies, eds.) pp. 460-74 (1999) (ASM Press, Washington, D.C.); H. Schweizer, Vectors to express foreign genes and techniques to monitor gene expression for Pseudomonads, Current Opinion in Biotechnology, 12:439-445 (2001); and R. Slater & R. Williams, The Expression of Foreign DNA in Bacteria, in Molecular Biology and Biotechnology (J. Walker & R. Rapley, eds.) pp. 125-54 (2000) (The Royal Society of Chemistry, Cambridge, UK).

A promoter having the nucleotide sequence of a promoter native to the selected bacterial host cell can also be used to control expression of the transgene encoding the target peptide, e.g., a Pseudomonas anthranilate or benzoate operon promoter (Pant, Pben). Tandem promoters may also be used in which more than one promoter is covalently attached to another, whether the same or different in sequence, e.g., a Pant-Pben tandem promoter (interpromoter hybrid) or a Plac-Plac tandem promoter.

Regulated promoters utilize promoter regulatory proteins in order to control transcription of the gene of which the promoter is a part. Where a regulated promoter is used herein, a corresponding promoter regulatory protein will also be part of an expression system according to the present invention. Examples of promoter regulatory proteins include: activator proteins, e.g., E. coli catabolite activator protein, MalT protein; AraC family transcriptional activators; repressor proteins, e.g., E. coli LacI proteins; and dual-faction regulatory proteins, e.g., E. coli NagC protein. Many regulated-promoter/promoter-regulatory-protein pairs are known in the art.

Promoter regulatory proteins interact with an effector compound, i.e. a compound that reversibly or irreversibly associates with the regulatory protein so as to enable the protein to either release or bind to at least one DNA transcription regulatory region of the gene that is under the control of the promoter, thereby permitting or blocking the action of a transcriptase enzyme in initiating transcription of the gene. Effector compounds are classified as either inducers or co-repressors, and these compounds include native effector compounds and gratuitous inducer compounds. Many regulated-promoter/promoter-regulatory-protein/effector-compound trios are known in the art. Although an effector compound can be used throughout the cell culture or fermentation, in a particular embodiment in which a regulated promoter is used, after growth of a desired quantity or density of host cell biomass, an appropriate effector compound is added to the culture in order to directly or indirectly result in expression of the desired target gene(s).

By way of example, where a lac family promoter is utilized, a lacI gene, or derivative thereof such as a lacI^{Q} or lacI^{Q1} gene, can also be present in the system. The lacI gene, which is (normally) a constitutively expressed gene, encodes the Lac repressor protein (LacI protein) which binds to the lac operator of these promoters. Thus, where a lac family promoter is utilized, the lacI gene can also be included and expressed in the expression system. In the case of the lac promoter family members, e.g., the tac promoter, the effector compound is an inducer, preferably a gratuitous inducer such as IPTG (isopropyl-β-D-1-thiogalactopyranoside, also called “isopropylthiogalactoside”).

In a particular embodiment, a lac or tac family promoter is utilized in the present invention, including Plac, Ptac, Ptrc, PtacII, PlacUV5, lpp-PlacUV5, lpp-lac, nprM-lac, T7lac, T5lac, T3lac, and Pmac.

Other Elements

Other regulatory elements can be included in an expression construct, including lacO sequences. Such elements include, but are not limited to, for example, transcriptional enhancer sequences, translational enhancer sequences, other promoters, activators, translational start and stop signals, transcription terminators, cistronic regulators, polycistronic regulators, tag sequences, such as nucleotide sequence “tags” and “tag” peptide coding sequences, which facilitates identification, separation, purification, or isolation of an expressed peptide, including His-tag, Flag-tag, T7-tag, S-tag, HSV-tag, B-tag, Strep-tag, polyarginine, polycysteine, polyphenylalanine, polyaspartic acid, (Ala-Trp-Trp-Pro)n, thioredoxin, beta-galactosidase, chloramphenicol acetyltransferase, cyclomaltodextrin gluconotransferase, CTP:CMP-3-deoxy-D-manno-octulosonate cytidyltransferase, trpE or trpLE, avidin, streptavidin, T7 gene 10, T4 gp55, Staphylococcal protein A, streptococcal protein G, GST, DHFR, CBP, MBP, galactose binding domain, Calmodulin binding domain, GFP, KSI, c-myc, ompT, ompA, pelB, NusA, ubiquitin, and hemosylin A.

In one embodiment, the nucleic acid construct further comprises a tag sequence adjacent to the coding sequence for the recombinant peptide of interest, or linked to a coding sequence for a viral capsid. In one embodiment, this tag sequence allows for purification of the protein. The tag sequence can be an affinity tag, such as a hexa-histidine affinity tag. In another embodiment, the affinity tag can be a glutathione-S-transferase molecule. The tag can also be a fluorescent molecule, such as YFP or GFP, or analogs of such fluorescent proteins. The tag can also be a portion of an antibody molecule, or a known antigen or ligand for a known binding partner useful for purification.

The present invention can include, in addition to the capsid-recombinant peptide coding sequence, the following regulatory elements operably linked thereto: a promoter, a ribosome binding site (RBS), a transcription terminator, translational start and stop signals. Useful RBSs can be obtained from any of the species useful as host cells in expression systems according to the present invention, preferably from the selected host cell. Many specific and a variety of consensus RBSs are known, e.g., those described in and referenced by D. Frishman et al., Starts of bacterial genes: estimating the reliability of computer predictions, Gene 234(2):257-65 (8 Jul. 1999); and B. E. Suzek et al., A probabilistic method for identifying start codons in bacterial genomes, Bioinformatics 17(12):1123-30 (December 2001). In addition, either native or synthetic RBSs may be used, e.g., those described in: EP 0207459 (synthetic RBSs); O. Ikehata et al., Primary structure of nitrile hydratase deduced from the nucleotide sequence of a Rhodococcus species and its expression in Escherichia coli, Eur. J. Biochem. 181(3):563-70 (1989) (native RBS sequence of AAGGAAG). Further examples of methods, vectors, and translation and transcription elements, and other elements useful in the present invention are described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to Wilcox.

Vectors

Transcription of the DNA encoding the enzymes of the present invention by a Pseudomonad host can further be increased by inserting an enhancer sequence into the vector or plasmid. Typical enhancers are cis-acting elements of DNA, usually about from 10 to 300 bp in size that act on the promoter to increase its transcription.

Generally, the recombinant expression vectors will include origins of replication and selectable markers permitting transformation of the Pseudomonad host cell, e.g., the capsid-recombinant peptide fusion peptides of the present invention, and a promoter derived from a highly-expressed gene to direct transcription of a downstream structural sequence. Such promoters have been described above. The heterologous structural sequence is assembled in appropriate phase with translation initiation and termination sequences. Optionally, and in accordance with the present invention, the heterologous sequence can encode a fusion peptide including an N-terminal identification peptide imparting desired characteristics, e.g., stabilization or simplified purification of expressed recombinant product.

Useful expression vectors for use with P. fluorescens in expressing capsid-recombinant peptide fusion peptides are constructed by inserting a structural DNA sequence encoding a desired target peptide fused with a capsid peptide together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable hosts for transformation in accordance with the present disclosure include various species within the genera Pseudomonas, and, in particular, the host cell strain of Pseudomonas fluorescens.

Vectors are known in the art as useful for expressing recombinant proteins in host cells, and any of these may be modified and used for expressing the fusion products according to the present invention. Such vectors include, e.g., plasmids, cosmids, and phage expression vectors. Examples of useful plasmid vectors that can be modified for use on the present invention include, but are not limited to, the expression plasmids pBBR1MCS, pDSK519, pKT240, pML122, pPS10, RK2, RK6, pRO1600, and RSF110. Further examples can include pALTER-Ex1, pALTER-Ex2, pBAD/His, pBAD/Myc-His, pBAD/gIII, pCal-n, pCal-n-EK, pCal-c, pCal-Kc, pcDNA 2.1, pDUAL, pET-3a-c, pET 9a-d, pET-11a-d, pET-12a-c, pET-14b, pET15b, pET-16b, pET-17b, pET-19b, pET-20b(+), pET-21a-d(+), pET-22b(+), pET-23a-d(+), pET24a-d(+), pET-25b(+), pET-26b(+), pET-27b(+), pET28a-c(+), pET-29a-c(+), pET-30a-c(+), pET31b(+), pET-32a-c(+), pET-33b(+), pET-34b(+), pET35b(+), pET-36b(+), pET-37b(+), pET-38b(+), pET-39b(+), pET-40b(+), pET-41a-c(+), pET-42a-c(+), pET-43a-c(+), pETBlue-1, pETBlue-2, pETBlue-3, pGEMEX-1, pGEMEX-2, pGEX1λT, pGEX-2T, pGEX-2TK, pGEX-3×, pGEX-4T, pGEX-5×, pGEX-6P, pHAT10/11/12, pHAT20, pHAT-GFPuv, pKK223-3, pLEX, pMAL-c2X, pMAL-c2E, pMAL-c2g, pMAL-p2X, pMAL-p2E, pMAL-p2G, pProEX HT, pPROLar.A, pPROTet.E, pQE-9, pQE-16, pQE-30/31/32, pQE-40, pQE-50, pQE-70, pQE-80/81/82L, pQE-100, pRSET, and pSE280, pSE380, pSE420, pThioHis, pTrc99A, pTrcHis, pTrcHis2, pTriEx-1, pTriEx-2, pTrxFus. Other examples of such useful vectors include those described by, e.g.: N. Hayase, in Appl. Envir. Microbiol. 60(9):3336-42 (September 1994); A. A. Lushnikov et al., in Basic Life Sci. 30:657-62 (1985); S. Graupner & W. Wackemagel, in Biomolec. Eng. 17(1):11-16. (October 2000); H. P. Schweizer, in Curr. Opin. Biotech. 12(5):439-45 (October 2001); M. Bagdasarian & K. N. Timmis, in Curr. Topics Microbiol. Immunol. 96:47-67 (1982); T. Ishii et al., in FEMS Microbiol. Lett. 116(3):307-13 (Mar. 1, 1994); I. N. Olekhnovich & Y. K. Fomichev, in Gene 140(1):63-65 (Mar. 11, 1994); M. Tsuda & T. Nakazawa, in Gene 136(1-2):257-62 (Dec. 22, 1993); C. Nieto et al., in Gene 87(1):145-49 (Mar. 1, 1990); J. D. Jones & N. Gutterson, in Gene 61(3):299-306 (1987); M. Bagdasarian et al., in Gene 16(1-3):237-47 (December 1981); H. P. Schweizer et al., in Genet. Eng. (NY) 23:69-81 (2001); P. Mukhopadhyay et al., in J. Bact. 172(1):477-80 (January 1990); D. O. Wood et al., in J. Bact. 145(3):1448-51 (March 1981); and R. Holtwick et al., in Microbiology 147(Pt 2):337-44 (February 2001).

Further examples of expression vectors that can be useful in Pseudomonas host cells include those listed in Table 2 as derived from the indicated replicons.

TABLE 2
SOME EXAMPLES OF USEFUL EXPRESSION VECTORS

| Replicon | Vector(s) |
|---|---|
| pPS10 | pCN39, pCN51 |
| RSF1010 | pKT261-3 |
| | pMMB66EH |
| | pEB8 |
| | pPLGN1 |
| | pMYC1050 |
| RK2/RP1 | pRK415 |
| | pJB653 |
| pRO1600 | pUCP |
| | pBSP |

The expression plasmid, RSF1010, is described, e.g., by F. Heffron et al., in Proc. Nat'l Acad. Sci. USA 72(9):3623-27 (September 1975), and by K. Nagahari & K. Sakaguchi, in J. Bact. 133(3):1527-29 (March 1978). Plasmid RSF1010 and derivatives thereof are particularly useful vectors in the present invention. Exemplary, useful derivatives of RSF1010, which are known in the art, include, e.g., pKT212, pKT214, pKT231 and related plasmids, and pMYC1050 and related plasmids (see, e.g., U.S. Pat. Nos. 5,527,883 and 5,840,554 to Thompson et al.), such as, e.g., pMYC1803. Plasmid pMYC1803 is derived from the RSF1010-based plasmid pTJS260 (see U.S. Pat. No. 5,169,760 to Wilcox), which carries a regulated tetracycline resistance marker and the replication and mobilization loci from the RSF1010 plasmid. Other exemplary useful vectors include those described in U.S. Pat. No. 4,680,264 to Puhler et al.

In a one embodiment, an expression plasmid is used as the expression vector. In another embodiment, RSF1010 or a derivative thereof is used as the expression vector. In still another embodiment, pMYC1050 or a derivative thereof, or pMYC1803 or a derivative thereof, is used as the expression vector.

The Champion™ pET expression system provides a high level of protein production. Expression is induced from the strong T7lac promoter. This system takes advantage of the high activity and specificity of the bacteriophage T7 RNA polymerase for high level transcription of the gene of interest. The lac operator located in the promoter region provides tighter regulation than traditional T7-based vectors, improving plasmid stability and cell viability (Studier, F. W. and B. A. Moffatt (1986) J Molecular Biology 189(1): 113-30; Rosenberg, et al. (1987) Gene 56(1): 125-35). The T7 expression system uses the T7 promoter and T7 RNA polymerase (T7 RNAP) for high-level transcription of the gene of interest. High-level expression is achieved in T7 expression systems because the T7 RNAP is more processive than native E. coli RNAP and is dedicated to the transcription of the gene of interest. Expression of the identified gene is induced by providing a source of T7 RNAP in the host cell. This is accomplished by using a BL21 E. coli host containing a chromosomal copy of the T7 RNAP gene. The T7 RNAP gene is under the control of the lacUV5 promoter which can be induced by IPTG. T7 RNAP is expressed upon induction and transcribes the gene of interest.

The pBAD expression system allows tightly controlled, titratable expression of recombinant protein through the presence of specific carbon sources such as glucose, glycerol and arabinose (Guzman, et al. (1995) J Bacteriology 177(14): 4121-30). The pBAD vectors are uniquely designed to give precise control over expression levels. Heterologous gene expression from the pBAD vectors is initiated at the araBAD promoter. The promoter is both positively and negatively regulated by the product of the araC gene. AraC is a transcriptional regulator that forms a complex with L-arabinose. In the absence of L-arabinose, the AraC dimer blocks transcription. For maximum transcriptional activation two events are required: (i) L-arabinose binds to AraC allowing transcription to begin. (ii.) The cAMP activator protein (CAP)-cAMP complex binds to the DNA and stimulates binding of AraC to the correct location of the promoter region.

The trc expression system allows high-level, regulated expression in E. coli from the trc promoter. The trc expression vectors have been optimized for expression of eukaryotic genes in E. coli. The trc promoter is a strong hybrid promoter derived from the tryptophane (trp) and lactose (lac) promoters. It is regulated by the lacO operator and the product of the lacIQ gene (Brosius, J. (1984) Gene 27(2): 161-72).

### III. Expression of Virus Like Particles in Pseudomonads

The present invention also provides a process for producing a recombinant peptide. The process includes:
a) providing a Pseudomonad cell;
b) providing a nucleic acid encoding a fusion peptide; wherein the fusion is of a recombinant peptide and an icosahedral capsid;
c) expressing the nucleic acid in the Pseudomonad cell, wherein the expression in the cell provides for in vivo assembly of the fusion peptide into virus like particles; and
d) isolating the virus like particles.

Peptides may be expressed as single-copy peptide inserts within a capsid peptide (i.e. expressed as individual inserts from recombinant capsid peptide coding sequences that are mono-cistronic for the peptide) or may be expressed as di-, tri-, or multi-copy peptide inserts (i.e. expressed as concatemeric inserts from recombinant capsid peptide coding sequences that are poly-cistronic for the peptide; the concatemeric insert(s) may contain multiple copies of the same exogenous peptide of interest or may contain copies of different exogenous peptides of interest). Concatemers may be homo- or hetero-concatemers.

In one embodiment, the isolated virus like particle can be administered to a human or animal in a vaccine strategy.

In another embodiment, the nucleic acid construct can be co-expressed with another nucleic acid encoding a wild type capsid. In a particular embodiment, the co-expressed capsid/capsid-recombinant peptide fusion particles assemble in vivo to form a chimeric virus like particle. The chimeric VLP is a virus like particle including capsids or capsid-peptide fusions encoded by at least two different nucleic acid constructs.

In still another embodiment, the nucleic acid construct can be co-expressed with another nucleic acid encoding a different capsid-recombinant peptide fusion particle. In a particular embodiment, the co-expressed capsid fusion particles will assemble in vivo to form a chimeric virus like particle.

In still another embodiment, a second nucleic acid, which is designed to express a different peptide, such as a chaperone protein, can be expressed concomitantly with the nucleic acid encoding the fusion peptide.

The Pseudomonad cells, capsids, and recombinant peptides useful for the present invention are discussed above.

In one embodiment, the process produces at least 0.1 g/L protein in the form of VLPs. In another embodiment, the process produces 0.1 to 10 g/L protein in the form of VLPs. In subembodiments, the process produces at least about 0.2, 0.3, 0.4, 0.5, 0.6, 0.7, 0.8, 0.9 or 1.0 g/L protein in the form of VLPs or cage structures. In one embodiment, the total recombinant protein produced is at least 1.0 g/L. In some embodiments, the amount of VLP protein produced is at least about 5%, about 10%, about 15%, about 20%, about 25%, about 30%, about 40%, about 50%, about 60%, about 70%, about 80%, about 90%, about 95% or more of total recombinant protein produced.

In one embodiment, the process produces at least 0.1 g/L pre-formed VLPs or cage structures. In another embodiment, the process produces 0.1 to 10 g/L pre-formed VLPs in the cell. In another embodiment, the process produces 0.1 to 10 g/L pre-formed cage structures in the cell. In subembodiments, the process produces at least about 0.2, 0.3, 0.4, 0.5, 0.6, 0.7, 0.8, 0.9 or 1.0 g/L pre-formed VLPs. In one embodiment, the total pre-formed VLP protein produced is at least 1.0 g/L. In subembodiments, the total VLP protein produced can be at least about 2.0, 3.0, 4.0, 5.0, 6.0, 7.0, 8.0, 9.0, 10.0, 15.0, 20.0 or 50.0 g/L. In some embodiments, the amount of VLP protein produced is at least about 5%, about 10%, about 15%, about 20%, about 25%, or more of total recombinant protein produced.

In another embodiment, more than 50% of the expressed, transgenic peptide, peptide, protein, or fragment thereof produced can be produced in a renaturable form in host cell. In another embodiment about 60%, 70%, 75%, 80%, 85%, 90%, 95% of the expressed protein is obtained in or can be renatured into active form.

The process of the invention can also lead to increased yield of recombinant protein. In one embodiment, the process produces recombinant protein as 5, 10, 15, 20, 25, 30, 40 or 50, 55, 60, 65, 70, or 75% of total cell protein (tcp). “Percent total cell protein” is the amount of peptide in the host cell as a percentage of aggregate cellular protein. The determination of the percent total cell protein is well known in the art.

In a particular embodiment, the host cell can have a recombinant peptide, peptide, protein, or fragment thereof expression level of at least 1% tcp and a cell density of at least 40 g/L, when grown (i.e. within a temperature range of about 4° C. to about 55° C., inclusive) in a mineral salts medium. In a particular embodiment, the expression system will have a recombinant protein of peptide expression level of at least 5% tcp and a cell density of at least 40 g/L, when grown (i.e. within a temperature range of about 4° C. to about 55° C., inclusive) in a mineral salts medium at a fermentation scale of at least 10 Liters.

In a separate embodiment, a portion of the expressed viral capsid operably linked to a peptide of interest is formed in an insoluble aggregate in the cell. In one embodiment, the peptide of interest can be renatured from the insoluble aggregate.

Cleavage of Peptide of Interest

In one embodiment, the process further provides: e) cleaving the fusion product to separate the recombinant peptide from the capsid.

A cleavable linkage sequence can be included between the viral protein and the recombinant peptide. Examples of agents that can cleave such sequences include, but are not limited to chemical reagents such as acids (HCl, formic acid), CNBr, hydroxylamine (for asparagine-glycine), 2-Nitro-5-thiocyanobenzoate, O-Iodosobenzoate, and enzymatic agents, such as endopeptidases, endoproteases, trypsin, clostripain, and Staphylococcal protease.

Cleavable linkage sequences are well known in the art. In the present invention, any cleavable linkage sequence recognized by cleavage agents, including dipeptide cleavage sequences such as Asp-Pro, can be utilized.

Expression

The process of the invention optimally leads to increased production of recombinant peptide in a host cell. The increased production alternatively can be an increased level of active peptide per gram of protein produced, or per gram of host protein. The increased production can also be an increased level of recoverable peptide, such as soluble protein, produced per gram of recombinant or per gram of host cell protein. The increased production can also be any combination of increased total level and increased active or soluble level of protein.

The improved expression of recombinant protein can be through expression of the protein inserted in VLPs. In certain embodiments, at least 60, at least 70, at least 80, at least 90, at least 100, at least 110, at least 120, at least 130, at least 140, at least 150, at least 160, at least 170, or at least 180 copies of a peptide of interest is expressed in each VLP. The VLPs can be produced and recovered from the cytoplasm, periplasm or extracellular medium of the host cell.

In another embodiment, the peptide can be insoluble in the cell. In certain embodiments, the insoluble peptide is produced in a particle formed of multiple capsids but not forming a native-type VLP. For example, a cage structure of as few as 3 viral capsids can be formed. In certain embodiments, the capsid structure includes more than one copy of a peptide of interest and in certain embodiments, includes at least ten, at least 20, or at least 30 copies.

The peptide or viral capsid sequence can also include one or more targeting sequences or sequences to assist purification. These can be an affinity tag. These can also be targeting sequences directing the assembly of capsids into a VLP.

Cell Growth

Transformation of the Pseudomonas host cells with the vector(s) may be performed using any transformation methodology known in the art, and the bacterial host cells may be transformed as intact cells or as protoplasts (i.e. including cytoplasts). Exemplary transformation methodologies include poration methodologies, e.g., electroporation, protoplast fusion, bacterial conjugation, and divalent cation treatment, e.g., calcium chloride treatment or CaCl/Mg2+ treatment, or other well known methods in the art. See, e.g., Morrison, J. Bact., 132:349-351 (1977); Clark-Curtiss & Curtiss, Methods in Enzymology, 101:347-362 (Wu et al., eds, 1983), Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

As used herein, the term “fermentation” includes both embodiments in which literal fermentation is employed and embodiments in which other, non-fermentative culture modes are employed. Fermentation may be performed at any scale. In one embodiment, the fermentation medium may be selected from among rich media, minimal media, and mineral salts media; a rich medium may be used, but is preferably avoided. In another embodiment either a minimal medium or a mineral salts medium is selected.

In still another embodiment, a minimal medium is selected. In yet another embodiment, a mineral salts medium is selected. Mineral salts media are particularly preferred.

Mineral salts media consists of mineral salts and a carbon source such as, e.g., glucose, sucrose, or glycerol. Examples of mineral salts media include, e.g., M9 medium, Pseudomonas medium (ATCC 179), Davis and Mingioli medium (see, B D Davis & E S Mingioli (1950) in J. Bact. 60:17-28). The mineral salts used to make mineral salts media include those selected from among, e.g., potassium phosphates, ammonium sulfate or chloride, magnesium sulfate or chloride, and trace minerals such as calcium chloride, borate, and sulfates of iron, copper, manganese, and zinc. No organic nitrogen source, such as peptone, tryptone, amino acids, or a yeast extract, is included in a mineral salts medium. Instead, an inorganic nitrogen source is used and this may be selected from among, e.g., ammonium salts, aqueous ammonia, and gaseous ammonia. A preferred mineral salts medium will contain glucose as the carbon source. In comparison to mineral salts media, minimal media can also contain mineral salts and a carbon source, but can be supplemented with, e.g., low levels of amino acids, vitamins, peptones, or other ingredients, though these are added at very minimal levels.

The high cell density culture can start as a batch process which is followed by a two-phase fed-batch cultivation. After unlimited growth in the batch part, growth can be controlled at a reduced specific growth rate over a period of 3 doubling times in which the biomass concentration can increased several fold. Further details of such cultivation procedures is described by Riesenberg, D.; Schulz, V.; Knorre, W. A.; Pohl, H. D.; Korz, D.; Sanders, E. A.; Ross, A.; Deckwer, W. D. (1991) “High cell density cultivation of Escherichia coli at controlled specific growth rate” J Biotechnol: 20(1) 17-27.

The expression system according to the present invention can be cultured in any fermentation format. For example, batch, fed-batch, semi-continuous, and continuous fermentation modes may be employed herein.

The expression systems according to the present invention are useful for transgene expression at any scale (i.e. volume) of fermentation. Thus, e.g., microliter-scale, centiliter scale, and deciliter scale fermentation volumes may be used; and 1 Liter scale and larger fermentation volumes can be used. In one embodiment, the fermentation volume will be at or above 1 Liter. In another embodiment, the fermentation volume will be at or above 5 Liters, 10 Liters, 15 Liters, 20 Liters, 25 Liters, 50 Liters, 75 Liters, 100 Liters, 200 Liters, 500 Liters, 1,000 Liters, 2,000 Liters, 5,000 Liters, 10,000 Liters or 50,000 Liters.

In the present invention, growth, culturing, and/or fermentation of the transformed host cells is performed within a temperature range permitting survival of the host cells, preferably a temperature within the range of about 4° C. to about 55° C., inclusive. Thus, e.g., the terms “growth” (and “grow,” “growing”), “culturing” (and “culture”), and “fermentation” (and “ferment,” “fermenting”), as used herein in regard to the host cells of the present invention, inherently means “growth,” “culturing,” and “fermentation,” within a temperature range of about 4° C. to about 55° C., inclusive. In addition, “growth” is used to indicate both biological states of active cell division and/or enlargement, as well as biological states in which a non-dividing and/or non-enlarging cell is being metabolically sustained, the latter use of the term “growth” being synonymous with the term “maintenance.”

Cell Density

An additional advantage in using Pseudomonas fluorescens in expressing recombinant peptides encased in VLPs includes the ability of Pseudomonas fluorescens to be grown in high cell densities compared to E. coli or other bacterial expression systems. To this end, Pseudomonas fluorescens expressions systems according to the present invention can provide a cell density of about 20 g/L or more. The Pseudomonas fluorescens expressions systems according to the present invention can likewise provide a cell density of at least about 70 g/L, as stated in terms of biomass per volume, the biomass being measured as dry cell weight.

In one embodiment, the cell density will be at least 20 g/L. In another embodiment, the cell density will be at least 25 g/L, 30 g/L, 35 g/L, 40 g/L, 45 g/L, 50 g/L, 60 g/L, 70 g/L, 80 g/L, 90 g/L, 100 g/L, 110 g/L, 120 g/L, 130 g/L, 140 g/L, or at least 150 g/L.

In another embodiments, the cell density at induction will be between 20 g/L and 150 g/L; 20 g/L and 120 g/L; 20 g/L and 80 g/L; 25 g/L and 80 g/L; 30 g/L and 80 g/L; 35 g/L and 80 g/L; 40 g/L and 80 g/L; 45 g/L and 80 g/L; 50 g/L and 80 g/L; 50 g/L and 75 g/L; 50 g/L and 70 g/L; 40 g/L and 80 g/L.

Isolation of VLP or Peptide of Interest

In certain embodiments, the invention provides a process for improving the recovery of peptides of interest by protection of the peptide during expression through linkage and co-expression with a viral capsid. In certain embodiments, the viral capsid fusion forms a VLP, which can be readily separated from the cell lysate.

The proteins of this invention may be isolated purified to substantial purity by standard techniques well known in the art, including, but not limited to, ammonium sulfate or ethanol precipitation, acid extraction, anion or cation exchange chromatography, phosphocellulose chromatography, hydrophobic interaction chromatography, affinity chromatography, nickel chromatography, hydroxylapatite chromatography, reverse phase chromatography, lectin chromatography, preparative electrophoresis, detergent solubilization, selective precipitation with such substances as column chromatography, immunopurification methods, and others. For example, proteins having established molecular adhesion properties can be reversibly fused a ligand. With the appropriate ligand, the protein can be selectively adsorbed to a purification column and then freed from the column in a relatively pure form. The fused protein is then removed by enzymatic activity. In addition, protein can be purified using immunoaffinity columns or Ni-NTA columns. General techniques are further described in, for example, R. Scopes, Protein Purification: Principles and Practice, Springer-Verlag: N.Y. (1982); Deutscher, Guide to Protein Purification, Academic Press (1990); U.S. Pat. No. 4,511,503; S. Roe, Protein Purification Techniques: A Practical Approach (Practical Approach Series), Oxford Press (2001); D. Bollag, et al., Protein Methods, Wiley-Lisa, Inc. (1996); AK Patra et al., Protein Expr Purif, 18(2): p/182-92 (2000); and R. Mukhija, et al., Gene 165(2): p. 303-6 (1995). See also, for example, Ausubel, et al. (1987 and periodic supplements); Deutscher (1990) “Guide to Protein Purification,” Methods in Enzymology vol. 182, and other volumes in this series; Coligan, et al. (1996 and periodic Supplements) Current Protocols in Protein Science Wiley/Greene, NY; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See also, for example. Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression & Protein Purification System QUIAGEN, Inc., Chatsworth, Calif.

Similarly, the virus-like particles or cage-like structures can be isolated and/or purified to substantial purity by standard techniques well known in the art. Techniques for isolation of VLPs, include, in addition to those described above, precipitation techniques such as polyethylene glycol or salt precitipation. Separation techniques include anion or cation exchange chromatography, size exclusion chromatograph, phosphocellulose chromatography, hydrophobic interaction chromatography, affinity chromatography, nickel chromatography, hydroxylapatite chromatography, reverse phase chromatography, lectin chromatography, preparative electrophoresis, immunopurification methods, centrifugation, ultracentrifugation, density gradient centrifugation (for example, on a sucrose or on a cesium chloride (CsCl) gradient), ultrafiltration through a size exclusion filter, and any other protein isolation methods known in the art.

The invention can also improve recovery of active recombinant peptides. Levels of active protein can be measured, for example, by measuring the interaction between an identified and a parent peptide, peptide variant, segment-substituted peptide and/or residue-substituted peptide by any convenient in vitro or in vivo assay. Thus, in vitro assays can be used to determine any detectable interaction between an identified protein and a peptide of interest, e.g. between enzyme and substrate, between hormone and hormone receptor, between antibody and antigen, etc. Such detection can include the measurement of colorimetric changes, changes in radioactivity, changes in solubility, changes in molecular weight as measured by gel electrophoresis and/or gel exclusion processes, etc. In vivo assays include, but are not limited to, assays to detect physiological effects, e.g. weight gain, change in electrolyte balance, change in blood clotting time, changes in clot dissolution and the induction of antigenic response. Generally, any in vivo assay can be used so long as a variable parameter exists so as to detect a change in the interaction between the identified and the peptide of interest. See, for example, U.S. Pat. No. 5,834,250.

To release recombinant proteins from the periplasm, treatments involving chemicals such as chloroform (Ames et al. (1984) J. Bacteriol., 160: 1181-1183), guanidine-HCl, and Triton X-100 (Naglak and Wang (1990) Enzyme Microb. Technol., 12: 603-611) have been used. However, these chemicals are not inert and may have detrimental effects on many recombinant protein products or subsequent purification procedures. Glycine treatment of E. coli cells, causing permeabilization of the outer membrane, has also been reported to release the periplasmic contents (Ariga et al. (1989) J. Ferm. Bioeng., 68: 243-246). The most widely used methods of periplasmic release of recombinant protein are osmotic shock (Nosal and Heppel (1966) J. Biol. Chem., 241: 3055-3062; Neu and Heppel (1965) J. Biol. Chem., 240: 3685-3692), hen egg white (HEW)-lysozyme/ethylenediamine tetraacetic acid (EDTA) treatment (Neu and Heppel (1964) J. Biol. Chem., 239: 3893-3900; Witholt et al. (1976) Biochim. Biophys. Acta, 443: 534-544; Pierce et al. (1995) ICheme Research. Event, 2: 995-997), and combined HEW-lysozyme/osmotic shock treatment (French et al. (1996) Enzyme and Microb. Tech., 19: 332-338). The French method involves resuspension of the cells in a fractionation buffer followed by recovery of the periplasmic fraction, where osmotic shock immediately follows lysozyme treatment. The effects of overexpression of the recombinant protein, S. thermoviolaceus α-amylase, and the growth phase of the host organism on the recovery are also discussed.

Typically, these procedures include an initial disruption in osmotically-stabilizing medium followed by selective release in non-stabilizing medium. The composition of these media (pH, protective agent) and the disruption methods used (chloroform, HEW-lysozyme, EDTA, sonication) vary among specific procedures reported. A variation on the HEW-lysozyme/EDTA treatment using a dipolar ionic detergent in place of EDTA is discussed by Stabel et al. (1994) Veterinary Microbiol., 38: 307-314. For a general review of use of intracellular lytic enzyme systems to disrupt E. coli, see Dabora and Cooney (1990) in Advances in Biochemical Engineering/Biotechnology, Vol. 43, A. Fiechter, ed. (Springer-Verlag: Berlin), pp. 11-30.

Conventional methods for the recovery of recombinant protein from the cytoplasm, as soluble protein or refractile particles, involved disintegration of the bacterial cell by mechanical breakage. Mechanical disruption typically involves the generation of local cavitations in a liquid suspension, rapid agitation with rigid beads, sonication, or grinding of cell suspension (Bacterial Cell Surface Techniques, Hancock and Poxton (John Wiley & Sons Ltd, 1988), Chapter 3, p. 55).

HEW-lysozyme acts biochemically to hydrolyze the peptidoglycan backbone of the cell wall. The method was first developed by Zinder and Arndt (1956) Proc. Natl. Acad. Sci. USA, 42: 586-590, who treated E. coli with egg albumin (which contains HEW-lysozyme) to produce rounded cellular spheres later known as spheroplasts. These structures retained some cell-wall components but had large surface areas in which the cytoplasmic membrane was exposed. U.S. Pat. No. 5,169,772 discloses a method for purifying heparinase from bacteria comprising disrupting the envelope of the bacteria in an osmotically-stabilized medium, e.g., 20% sucrose solution using, e.g., EDTA, lysozyme, or an organic compound, releasing the non-heparinase-like proteins from the periplasmic space of the disrupted bacteria by exposing the bacteria to a low-ionic-strength buffer, and releasing the heparinase-like proteins by exposing the low-ionic-strength-washed bacteria to a buffered salt solution.

Many different modifications of these methods have been used on a wide range of expression systems with varying degrees of success (Joseph-Liazun et al. (1990) Gene, 86: 291-295; Carter et al. (1992) Bio/Technology, 10: 163-167). Efforts to induce recombinant cell culture to produce lysozyme have been reported. EP 0 155 189 discloses a means for inducing a recombinant cell culture to produce lysozymes, which would ordinarily be expected to kill such host cells by means of destroying or lysing the cell wall structure.

U.S. Pat. No. 4,595,658 discloses a method for facilitating externalization of proteins transported to the periplasmic space of E. coli. This method allows selective isolation of proteins that locate in the periplasm without the need for lysozyme treatment, mechanical grinding, or osmotic shock treatment of cells. U.S. Pat. No. 4,637,980 discloses producing a bacterial product by transforming a temperature-sensitive lysogen with a DNA molecule that codes, directly or indirectly, for the product, culturing the transformant under permissive conditions to express the gene product intracellularly, and externalizing the product by raising the temperature to induce phage-encoded functions. Asami et al. (1997) J. Ferment. and Bioeng., 83: 511-516 discloses synchronized disruption of E. coli cells by T4 phage infection, and Tanji et al. (1998) J. Ferment. and Bioeng., 85: 74-78 discloses controlled expression of lysis genes encoded in T4 phage for the gentle disruption of E. coli cells.

Upon cell lysis, genomic DNA leaks out of the cytoplasm into the medium and results in significant increase in fluid viscosity that can impede the sedimentation of solids in a centrifugal field. In the absence of shear forces such as those exerted during mechanical disruption to break down the DNA polymers, the slower sedimentation rate of solids through viscous fluid results in poor separation of solids and liquid during centrifugation. Other than mechanical shear force, there exist nucleolytic enzymes that degrade DNA polymer. In E. coli, the endogenous gene endA encodes for an endonuclease (molecular weight of the mature protein is approx. 24.5 kD) that is normally secreted to the periplasm and cleaves DNA into oligodeoxyribonucleotides in an endonucleolytic manner. It has been suggested that endA is relatively weakly expressed by E. coli (Wackernagel et al. (1995) Gene 154: 55-59).

Detection of the expressed protein is achieved by methods known in the art and include, for example, radioimmunoassays, Western blotting techniques or immunoprecipitation.

Certain proteins expressed in this invention may form insoluble aggregates (“inclusion bodies”). Several protocols are suitable for purification of proteins from inclusion bodies. For example, purification of inclusion bodies typically involves the extraction, separation and/or purification of inclusion bodies by disruption of the host cells, e.g., by incubation in a buffer of 50 mM TRIS/HCL pH 7.5, 50 mM NaCl, 5 mM MgCl.sub.2, 1 mM DTT, 0.1 mM ATP, and 1 mM PMSF. The cell suspension is typically lysed using 2-3 passages through a French Press. The cell suspension can also be homogenized using a Polytron (Brinkrnan Instruments) or sonicated on ice. Alternate methods of lysing bacteria are apparent to those of skill in the art (see, e.g., Sambrook et al., supra; Ausubel et al., supra).

If necessary, the inclusion bodies can be solubilized, and the lysed cell suspension typically can be centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art.

Alternatively, it is possible to purify the recombinant peptides from the host periplasm. After lysis of the host cell, when the recombinant protein is exported into the periplasm of the host cell, the periplasmic fraction of the bacteria can be isolated by cold osmotic shock in addition to other methods known to those skilled in the art. To isolate recombinant proteins from the periplasm, for example, the bacterial cells can be centrifuged to form a pellet. The pellet can be resuspended in a buffer containing 20% sucrose. To lyse the cells, the bacteria can be centrifuged and the pellet can be resuspended in ice-cold 5 mM MgSO.sub.4 and kept in an ice bath for approximately 10 minutes. The cell suspension can be centrifuged and the supernatant decanted and saved. The recombinant proteins present in the supernatant can be separated from the host proteins by standard separation techniques well known to those of skill in the art.

An initial salt fractionation can separate many of the unwanted host cell proteins (or proteins derived from the cell culture media) from the recombinant protein of interest. One such example can be ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of a recombinant protein can be used to isolated it from proteins of greater and lesser size using ultrafiltration through membranes of different pore size (for example, Amicon or Millipore membranes). As a first step, the protein mixture can be ultrafiltered through a membrane with a pore size that has a lower molecular weight cut-off than the molecular weight of the protein of interest. The retentate of the ultrafiltration can then be ultrafiltered against a membrane with a molecular cut off greater than the molecular weight of the protein of interest. The recombinant protein will pass through the membrane into the filtrate. The filtrate can then be chromatographed as described below.

Recombinant proteins can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Renaturation and Refolding

Insoluble protein can be renatured or refolded to generate secondary and tertiary protein structure conformation. Protein refolding steps can be used, as necessary, in completing configuration of the recombinant product. Refolding and renaturation can be accomplished using an agent that is known in the art to promote dissociation/association of proteins. For example, the protein can be incubated with dithiothreitol followed by incubation with oxidized glutathione disodium salt followed by incubation with a buffer containing a refolding agent such as urea.

Recombinant protein can also be renatured, for example, by dialyzing it against phosphate-buffered saline (PBS) or 50 mM Na-acetate, pH 6 buffer plus 200 mM NaCl. Alternatively, the protein can be refolded while immobilized on a column, such as the Ni NTA column by using a linear 6M-1M urea gradient in 500 mM NaCl, 20% glycerol, 20 mM Tris/HCl pH 7.4, containing protease inhibitors. The renaturation can be performed over a period of 1.5 hours or more. After renaturation the proteins can be eluted by the addition of 250 mM immidazole. Immidazole can be removed by a final dialyzing step against PBS or 50 mM sodium acetate pH 6 buffer plus 200 mM NaCl. The purified protein can be stored at 4° C. or frozen at −80° C.

Other methods include, for example, those that may be described in MH Lee et al., Protein Expr. Purif., 25(1): p. 166-73 (2002), W. K. Cho et al., J. Biotechnology, 77(2-3): p. 169-78 (2000), Ausubel, et al. (1987 and periodic supplements), Deutscher (1990) “Guide to Protein Purification,” Methods in Enzymology vol. 182, and other volumes in this series, Coligan, et al. (1996 and periodic Supplements) Current Protocols in Protein Science Wiley/Greene, NY, S. Roe, Protein Purification Techniques: A Practical Approach (Practical Approach Series), Oxford Press (2001); D. Bollag, et al., Protein Methods, Wiley-Lisa, Inc. (1996).

Active Peptide Analysis

Active proteins can have a specific activity of at least 20%, 30%, or 40%, and preferably at least 50%, 60%, or 70%, and most preferably at least 80%, 90%, or 95% that of the native peptide that the sequence is derived from. Further, the substrate specificity (k_{cat}/Kₘ) is optionally substantially similar to the native peptide. Typically, k_{cat}/Kₘ will be at least 30%, 40%, or 50%, that of the native peptide; and more preferably at least 60%, 70%, 80%, or 90%. Methods of assaying and quantifying measures of protein and peptide activity and substrate specificity (k_{cat}/Kₘ), are well known to those of skill in the art.

The activity of a recombinant peptide produced in accordance with the present invention by can be measured by any protein specific conventional or standard in vitro or in vivo assay known in the art. The activity of the Pseudomonas produced recombinant peptide can be compared with the activity of the corresponding native protein to determine whether the recombinant protein exhibits substantially similar or equivalent activity to the activity generally observed in the native peptide under the same or similar physiological conditions.

The activity of the recombinant protein can be compared with a previously established native peptide standard activity. Alternatively, the activity of the recombinant peptide can be determined in a simultaneous, or substantially simultaneous, comparative assay with the native peptide. For example, an in vitro assays can be used to determine any detectable interaction between a recombinant peptide and a target, e.g. between an expressed enzyme and substrate, between expressed hormone and hormone receptor, between expressed antibody and antigen, etc. Such detection can include the measurement of calorimetric changes, proliferation changes, cell death, cell repelling, changes in radioactivity, changes in solubility, changes in molecular weight as measured by gel electrophoresis and/or gel exclusion methods, phosphorylation abilities, antibody specificity assays such as ELISA assays, etc. In addition, in vivo assays include, but are not limited to, assays to detect physiological effects of the Pseudomonas produced peptide in comparison to physiological effects of the native peptide, e.g. weight gain, change in electrolyte balance, change in blood clotting time, changes in clot dissolution and the induction of antigenic response. Generally, any in vitro or in vivo assay can be used to determine the active nature of the Pseudomonas produced recombinant peptide that allows for a comparative analysis to the native peptide so long as such activity is assayable. Alternatively, the peptides produced in the present invention can be assayed for the ability to stimulate or inhibit interaction between the peptide and a molecule that normally interacts with the peptide, e.g. a substrate or a component of the signal pathway that the native protein normally interacts. Such assays can typically include the steps of combining the protein with a substrate molecule under conditions that allow the peptide to interact with the target molecule, and detect the biochemical consequence of the interaction with the protein and the target molecule. Assays that can be utilized to determine peptide activity are described, for example, in Ralph, P. J., et al. (1984) J. Immunol. 132:1858 or Saiki et al. (1981) J. Immunol. 127:1044, Steward, W. E. II (1980) The Interferon Systems. Springer-Verlag, Vienna and New York, Broxmeyer, H. E., et al. (1982) Blood 60:595, “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987, AK Patra et al., Protein Expr Purif, 18(2): p/182-92 (2000), Kodama et al., J. Biochem. 99: 1465-1472 (1986); Stewart et al., Proc. Nat'l Acad. Sci. USA 90: 5209-5213 (1993); (Lombillo et al., J. Cell Biol. 128:107-115 (1995); (Vale et al., Cell 42:39-50 (1985).

### EXAMPLES

In these examples, the cowpea chlorotic mottle virus (CCMV) has been used as a peptide carrier and Pseudomonas fluorescens has been used as the expression host. CCMV is a member of the bromovirus group of the Bromoviridae. Bromoviruses are 25-28 nm diameter icosahedral viruses with a four-component, positive sense, single-stranded RNA genome. RNA1 and RNA2 code for replicase enzymes. RNA3 codes for a protein involved in viral movement within plant hosts. RNA4 (a subgenomic RNA derived from RNA 3), i.e. sgRNA4, codes for the 20 kDa capsid (CP), SEQ ID NO:1.

Wild type CCMV capsid encoded by sgRNA4 (SEQ ID NO:1)
Met Ser Thr Val Gly Thr Gly Lys Leu Thr Arg Ala Gln Arg Arg Ala Ala Ala Arg
Lys Asn Lys Arg Asn Thr Arg Val Val Gln Pro Val Ile Val Glu Pro Ile Ala Ser
Gly Gln Gly Lys Ala Ile Lys Ala Trp Thr Gly Tyr Ser Val Ser Lys Trp Thr Ala
Ser Cys Ala Ala Ala Glu Ala Lys Val Thr Ser Ala Ile Tbr Ile Ser Leu Pro Asn
Glu Leu Ser Ser Glu Arg Asn Lys Gln Leu Lys Val Gly Arg Val Leu Leu Trp Leu
Gly Leu Leu Pro Ser Val Ser Gly Thr Val Lys Ser Cys Val Thr Glu Thr Gln Thr
Thr Ala Ala Ala Ser Phe Gln Val Ala Leu Ala Val Ala Asp Asn Ser Lys Asp Val
Val Ala Ala Met Tyr Pro Glu Ala Phe Lys Gly Ile Thr Leu Glu Gln Leu Thr Ala
Asp Leu Thr Ile Tyr Leu Tyr Ser Ser Ala Ala Leu Thr Glu Gly Asp Val Ile Val
His Leu Glu Val Glu His Val Arg Pro Thr Phe Asp Asp Ser Phe Thr Pro Val Tyr

Each CCMV particle contains up to about 180 copies of the CCMV CP. An exemplary DNA sequence encoding the CCMV CP is shown in SEQ ID NO: 21.

Exemplary DNA sequence encoding the CCMV CP (SEQ ID NO:21)
atg tct aca gtc gga aca ggg aag tta act cgt gca caa cga agg gct gcg gcc cgt aag aac
aag cgg aac act cgt gtg gtc caa cct gtt att gta gaa ccc atc gct tca ggc caa ggc aag
gct att aaa gca tgg acc ggt tac agc gta tcg aag tgg acc gcc tct tgc gcg gcc gcc gaa
gct aaa gta acc tcg gct ata act atc tct ctc cct aat gag cta tcg tcc gaa agg aac aag
cag ctc aag gta ggt aga gtt tta tta tgg ctt ggg ttg ctt ccc agt gtt agt ggc aca gtg
aaa tcc tgt gtt aca gag acg cag act act gct gct gcc tcc ttt cag gtg gca tta gct gtg
gcc gac aac tcg aaa gat gtt gtc gct gct atg tac ccc gag gcg ttt aag ggt ata acc ctt
gaa caa ctc acc gcg gat tta acg atc tac ttg tac agc agt gcg gct ctc act gag ggc gac
gtc atc gtg cat ttg gag gtt gag cat gtc aga cct acg ttt gac gac tct ttc act ccg gtg
tat tag

The crystal structure of CCMV has been solved. This structure provides a clearer picture of the capsid interactions that appear to be critical to particle stability and dynamics and has been helpful in guiding rational design of insertion sites. Previous studies have demonstrated that CCMV capsids can be genetically modified to carry heterologous peptides without interfering with their ability to form particles. A number of suitable insertion sites have been identified.

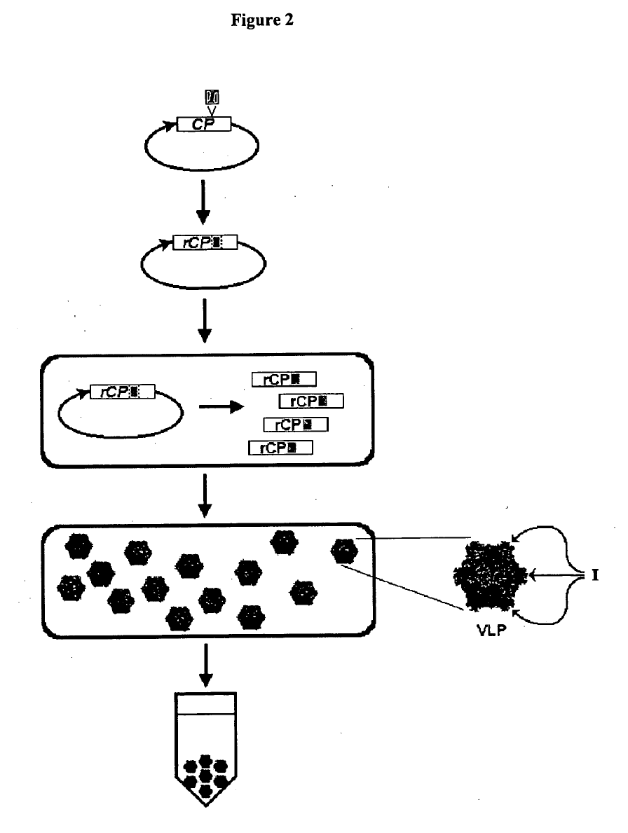

The general strategy followed for production of capsid-peptide fusion VLPs in P. fluorescens is diagrammed in FIG. 2. A total of up to about 180 copies of a heterologous peptide unit (whether individual peptide or concatemer) can be inserted into the CCMV particle if a single insertion site in the CCMV CP is used. Insertion sites identified within CCMV CP to date can accommodate peptides of various lengths. In addition, multimeric forms of the peptides can be inserted into insertion sites. Furthermore, multiple insertion sites can be used at the same time to express the same or different peptides in/on the same particle. The peptide inserts can be about 200 amino acid residues or less in length, more preferably up to or about 180, even more preferably up to or about 150, still more preferably up to or about 120, yet more preferably up to or about 100 amino acid residues in length. In a preferred embodiment, the peptide inserts will be about 5 or more amino acid residues in length. In a preferred embodiment, the peptide inserts will be about 5 to about 120, more preferably about 5 to about 100 amino acid residues in length.

Materials and Methods

Unless otherwise noted, standard techniques, vectors, control sequence elements, and other expression system elements known in the field of molecular biology are used for nucleic acid manipulation, transformation, and expression. Such standard techniques, vectors, and elements can be found, for example, in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); and Bukhari et al. (eds.), DNA Insertion Elements, Plasmids and Episomes (1977) (Cold Spring Harbor Laboratory Press, NY).

Plasmid Map Constructions

All plasmid maps were constructed using VECTORNTI (InforMax Inc., Frederick, Md., USA).

DNA Extractions

All plasmid DNA extractions from E. coli were performed using the mini, midi, and maxi kits from Qiagen (Germany) according to the manufacturer instructions.

Experimental Strategy

The following procedures were followed. P. fluorescens host cells were transformed with expression plasmids encoding chimeric viral capsid-target peptide insert fusions. Transformed cells were grown to the desired density and induced to express the chimeric viral capsid-peptide fusions. Cells were then lysed and their contents analyzed.

Construction of Modified CCMV-CP DNA to Add an Engineered Insertion Site

A DNA molecule containing the CCMV CP coding sequence was modified by inserting, in reading frame, a BamHI restriction enzyme recognition and cleavage site (gggatcctn), which introduced a tripeptide (Gly-Ile-Leu), into the native CCMV-CP amino acid sequence, between Asn129 and Ser130. Thus, the native CCMV-CP amino acid sequence (SEQ ID NO:1) was modified to form CCMV129-CP (SEQ ID NO:2).

CCMV-CP with added BamHI site inserted at codon 129 (CCMV129-CP)
(SEQ ID NO:2):
Met Ser Thr Val Gly Thr Gly Lys Leu Thr Arg Ala Gln Arg Arg Ala Ala Ala Arg
Lys Asn Lys Arg Asn Thr Arg Val Val Gln Pro Val Ile Val Glu Pro Ile Ala Ser
Gly Gln Gly Lys Ala Ile Lys Ala Trp Thr Gly Tyr Ser Val Ser Lys Trp Thr Ala
Ser Cys Ala Ala Ala Glu Ala Lys Val Thr Ser Ala Ile Thr Ile Ser Leu Pro Asn
Glu Leu Ser Ser Glu Arg Asn Lys Gln Leu Lys Val Gly Arg Val Leu Leu Trp Leu
Gly Leu Leu Pro Ser Val Ser Gly Thr Val Lys Ser Cys Val Thr Glu Thr Gln Thr
Thr Ala Ala Ala Ser Phe Gln Val Ala Leu Ala Val Ala Asp Asn Gly Ile Leu Ser
Lys Asp Val Val Ala Ala Met Tyr Pro Glu Ala Phe Lys Gly Ile Thr Leu Gln Gln
Leu Thr Ala Asp Leu Thr Ile Tyr Leu Tyr Ser Ser Ala Ala Leu Thr Glu Gly Asp
Val Ile Val His Leu Glu Val Glu His Val Arg Pro Thr Phe Asp Asp Ser Phe Thr
Pro Val Tyr

Primer CCMV-For (nucleic acid sequence: 5′-gactagtagg aggaaagaga tgtctacagt cgg-3′(SEQ ID NO:3)) was designed to add an ACTAGT SpeI restriction site and to add the consensus Shine-Dalgarno sequence to the CCMV-CP coding sequence. Primer CCMV-Rev (nucleic acid sequence: 5′-ccgctcgagt cattactaat acaccgg-3′ (SEQ ID NO:4)) was designed to add a CTCGAG XhoI restriction site and to introduce two stop codons to the CCMV-CP coding sequence. These two primers were used in a first PCR reaction with the DNA coding sequence of CCMV-CP.

Construction of CCMV63-CP DNA to Add an Engineered Insertion Site:

Restriction sites AscI and NotI were engineered onto CCMV-CP (SEQ ID NO:1) to serve as an insertion site. Recognition and cleavage sites for AscI (ggcgcgcc), NotI (gcggccgc), and additional nucleotides introduced a heptapeptide (Glu-Ala-Trp-Arg-Ala-Ala-Ala) onto CCMV-CP between residue Ala 60 and Ala 61. Hence, CCMV-CP was modified to form CCMV63-CP. In addition, residue Arg 26 was mutated to Cys 26 to add stability to assembled VLPs.

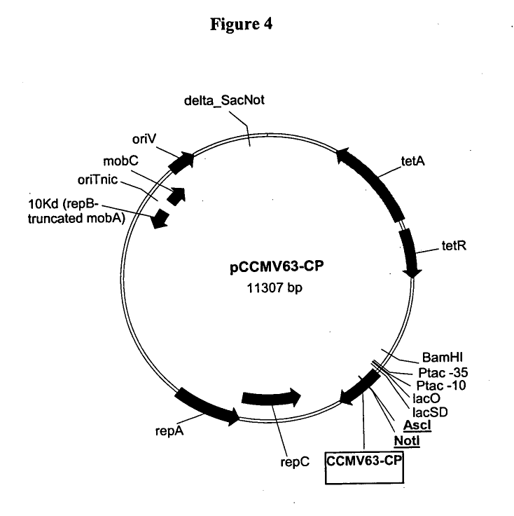

The plasmid map of pCCMV63-CP is shown in FIG. 4.

Sequence of CCMV63-CP ORF (SEQ ID NO:22):
atgtctacagtcggaacagggaagttaactcgtgcacaacgaagggctgcggcccgtaagaacaagcggaacacttgt
gtggtccaacctgttattgtagaacccatcgcttcaggccaaggcaaggctattaaagcatggaccggttacagcgta
tcgaagtggaccgcctcttgtgcggctgccgaagcttggcgcgccgcggccgctaaagtaacctcggctataactatc
tctctccctaatgagctatcgtccgaaaggaacaagcagctcaaggtaggtagagttttattatggcttgggttgctt
cccagtgttagtggcacagtgaaatcctgtgttacagagacgcagactactgctgctgcctcctttcaggtggcatta
gctgtggccgacaactcgaaagatgttgtcgctgctatgtaccccgaggcgtttaagggtataacccttgaacaactc
accgcggatttaacgatctacttgtacagcagtgcggctctcactgagggcgacgtcatcgtgcatttggaggttgag
catgtcagacctacgtttgacgactctttcactccggtgtattagtaatga

Construction of Double Insertion R26C-CCMV63/129-CP:

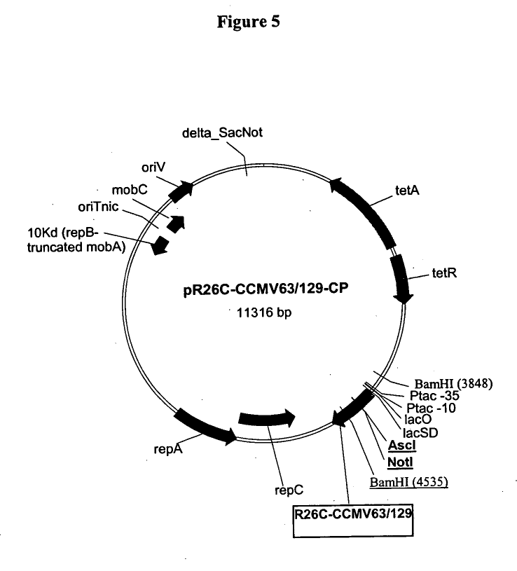

Restriction sites AscI and NotI were engineered onto CCMV129-CP (SEQ ID NO:2) to serve as the second insertion site. Recognition and cleavage sites for AscI (ggcgcgcc), NotI (gcggccgc), and additional nucleotides introduced a heptapeptide (Glu-Ala-Trp-Arg-Ala-Ala-Ala) onto CCMV129-CP between residue Ala 60 and Ala 61. Hence, CCMV129-CP was modified to form CCMV63/129-CP. In addition, residue Arg 26 was mutated to Cys 26 to add stability to assembled VLPs to create R26C—CCMV63/129-CP. The plasmid map of pR26C-CCMV63/129-CP is shown in FIG. 5.

Sequence of R26C-CCMV63/129-CP ORF (SEQ ID NO:23):
atgtctacagtcggaacagggaagttaactcgtgcacaacgaagggctgcggcccgtaagaacaagcggaacacttgt
gtggtccaacctgttattgtagaacccatcgcttcaggccaaggcaaggctattaaagcatggaccggttacagcgta
tcgaagtggaccgcctcttgtgcggctgccgaagcttggcgcgccgcggccgctaaagtaacctcggctataactatc
tctctccctaatgagctatcgtccgaaaggaacaagcagctcaaggtaggtagagttttattatggcttgggttgctt
cccagtgttagtggcacagtgaaatcctgtgttacagagacgcagactactgctgctgcctcctttcaggtggcatta
gctgtggccgacaacgggatcctgtcgaaagatgttgtcgctgctatgtaccccgaggcgtttaagggtataaccctt
gaacaactcaccgcggatttaacgatctacttgtacagcagtgcggctctcactgagggcgacgtcatcgtgcatttg
gaggttgagcatgtcagacctacgtttgacgactctttcactccggtgtattagtaatga

### Example 1

### Production of Peptide PD1 in CCMV VLPs in Pseudomonas

1.A. Construction of the Chimeric CCMV-PD1 Gene

A 20 amino acid antigenic peptide was selected for expression as an insert in the CCMV viral capsid. The antigenic peptide was unrelated to CCMV and to Pseudomonas fluorescens. An oligonucleotide encoding the peptide was amplified out of plasmid pCP7 Parvol DNA using primers Parvo-BamHI-F (nucleic acid sequence: 5′-cgggatcctg gacccggatg-3′ (SEQ ID NO:16)) and Parvo-BamI-R (nucleic acid sequence: 5′-cgggatcccc gggtctcttt c-3′ (SEQ ID NO:17)). (These primers were obtained from Integrated DNA Technologies, Inc., Coralville, Iowa, USA, hereinafter “IdtDNA.”) These primers amplified out a Canine parvovirus peptide coding sequence while adding BamHI restriction sites thereto at both ends for insertion into the CCMV129 coding sequence, at the BamHI restriction site thereof.

The PCR reactions were performed using a PTC225 thermocycler (MJ Research, South San Francisco, Calif., USA) according to the following protocol:

TABLE 4
PCR PROTOCOL

| Reaction Mix (100 μL total volume) | | Thermocycling Steps | | | |
|---|---|---|---|---|---|
| 10 μL | 10X PT HIFI buffer * | Step 1 | 1 Cycle | 2 min. | 94° C. |
| 4 μL | 50 mM MgSO₄* | | | 30 sec. | 94° C. |
| 2 μL | 10 mM dNTPs * | Step 2 | 35 Cycles | 30 sec. | 55° C. |
| 0.25 ng | Each Primer | | | 1 min. | 68° C. |
| 1-5 ng | Template DNA | Step 3 | 1 Cycle | 10 min. | 70° C. |
| 1 μL | PT HIFI Taq DNA Polymerase * | Step 4 | 1 Cycle | Maintain | 4° C. |
| Remainder | Distilled De-ionized H₂O (ddH₂O) | | | | |

* (from Invitrogen Corp, Carlsbad, CA, USA, hereinafter “Invitrogen”)

The DNA sequence was inserted into the CCMV129 shuttle plasmid, a plasmid that had been constructed from plasmid pESC (obtained from Stratagene Corp., LaJolla, Calif., USA) by inserting nucleic acid containing the CCMV129 CP DNA sequence therein, by use of SpeI and XhoI restriction enzymes. The PD1 peptide-encoding nucleic acid was inserted at the BamHI restriction site within the CCMV129 CDS, producing the CCMV129-PD1 shuttle plasmid.

PD1 CDS was also inserted into the CCMV129 CDS. As a result, the inserted PD1 coding sequence is: 5′-tgg gcc tgc cgc ggc acg gcc ggc tgg ccg ccg tcc ggc tgc acg gcg ccg tcc ggg tcg-3′ (SEQ ID NO:18), encoding a PD1 peptide whose amino acid sequence is: Trp Ala Cys Arg Gly Thr Ala Gly Trp Pro Pro Ser Gly Cys Thr Ala Pro Ser Gly Ser (SEQ ID NO:7). The PD1-coding nucleotide sequence is unrelated to Canine parvovirus.

1.B. Construction of a CCMV-PD1 Expression Plasmid

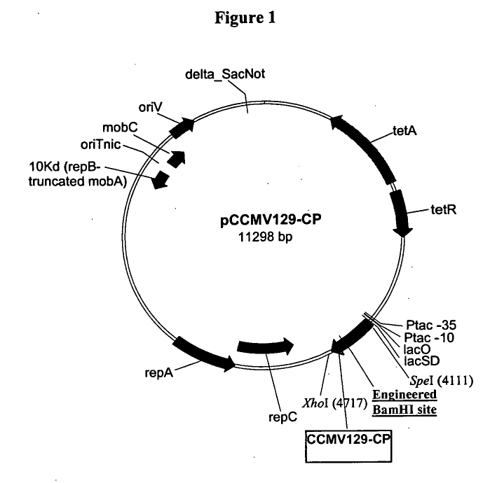

The CCMV129-PD1 shuttle plasmid was digested with SpeI and XhoI restriction enzymes. The fragment containing the chimeric CCMV129-PD1 DNA sequence was isolated by gel purification. It was then inserted into the pMYC1803 expression plasmid, in place of the buibui toxin gene, in operable attachment to a tac promoter, at the expression plasmid's SpeI and XhoI restriction sites. See FIG. 1. The resulting expression plasmid was screened by restriction enzyme digestion with SpeI and XhoI to verify the presence of the insert.

1.C. Plasmid Transformation into Pseudomonas Host Cells

The CCMV129-PD1 expression plasmid was transformed into Pseudomonas fluorescens MB214 host cells according to the following protocol. Host cells were thawed gradually in vials maintained on ice. For each transformation, 1 μL purified expression plasmid DNA was added to the host cells and the resulting mixture was swirled gently with a pipette tip to mix, and then incubated on ice for 30 min. The mixture was transferred to electroporation disposable cuvettes (BioRad Gene Pulser Cuvette, 0.2 cm electrode gap, cat no. 165-2086). The cuvettes were placed into a Biorad Gene Pulser pre-set at 200 Ohms, 25 μfarads, 2.25 kV. Cells were pulse cells briefly (about 1-2 sec). Cold LB medium was then immediately added and the resulting suspension was incubated at 30° C. for 2 hours. Cells were then plated on LB tet15 (tetracycline-supplemented LB medium) agar and grown at 30° C. overnight.

1.D. Shake-Flask Expression of CCMV-PD 1 Construct

One colony was picked from each plate and the picked sample was inoculated into 50 mL LB seed culture in a baffled shake flask. Liquid suspension cultures were grown overnight at 30° C. with 250 rpm shaking. 10 μL of each resulting seed culture was then used to inoculate 200 mL of shake-flask medium (i.e. yeast extracts and salt with trace elements, sodium citrate, and glycerol, pH 6.8) in a 1 liter baffled shake flask. Tetracycline was added for selection. Inoculated cultures were grown overnight at 30° C. with 250 rpm shaking and induced with IPTG for expression of the CCMV129-PD1 chimeric capsids.

I.E. Separation of Cell Culture Lysate into Soluble and Insoluble Fractions

1 mL aliquots from each shake-flask culture were then centrifuged to pellet the cells. Cell pellets were resuspended in 0.75 mL cold 50 mM Tris-HCl, pH 8.2, containing 2 mM EDTA. 0.1% volume of 10% TritonX-100 detergent was then added, followed by an addition of lysozyme to 0.2 mg/mL final concentration. Cells were then incubated on ice for 2 hours, at which time a clear and viscous cell lysate should be apparent.

To the lysates, 1/200 volume 1M MgCl₂ was added, followed by an addition of 1/200 volume 2 mg/mL DNAseI, and then incubation on ice for 1 hour, by which time the lysate should have become a much less viscous liquid. Treated lysates were then spun for 30 min at 4° C. at maximum speed in a tabletop centrifuge and the supernatants were decanted into clean tubes. The decanted supernatants are the “soluble” protein fractions. The remaining pellets were then resuspended in 0.75 mL TE buffer (10 mM Tris-Cl, pH 7.5, 1 mM EDTA). The resuspended pellets are the “insoluble” fractions.

1.F. SDS-PAGE and Western Blot Analysis of Soluble and Insoluble Protein Fractions

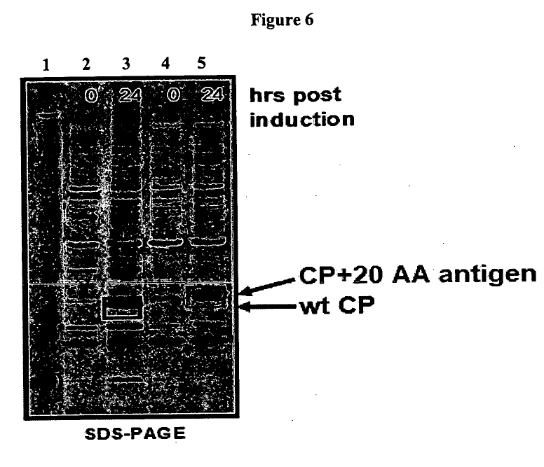

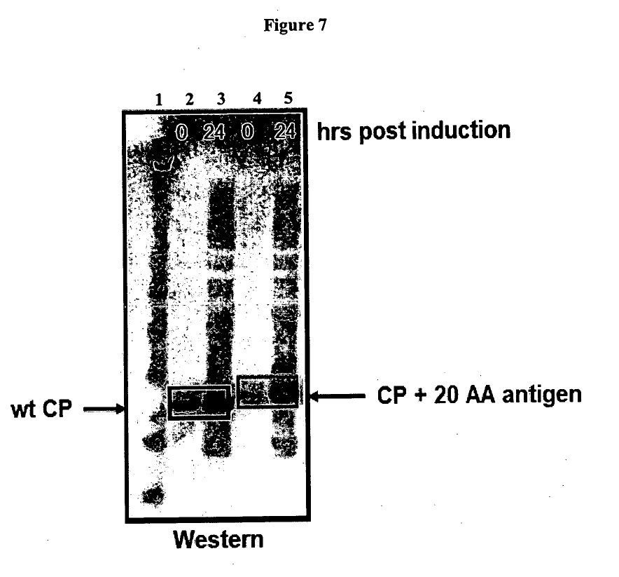

These “soluble” and “insoluble” fractions were then electrophoresed on NuPAGE 4-12% Bis-Tris gels (from Invitrogen, Cat. NP0323), having 1.0 mm×15 wells, according to manufacturer's specification. Gels were stained with SimplyBlue Safe Stain, (from Invitrogen, Cat. LC6060) and destained overnight with water. Western blot detection employed CCMV IgG (Accession No. AS0011 from DSMZ, Germany) and the WESTERN BREEZE kit (from Invitrogen, Cat. WB7105), following manufacturer's protocols. Results were positive for production of CCMV and specifically for production of chimeric CCMV CP (see FIGS. 6 and 7).

1.G. PEG Precipitation of Chimeric VLPs

Chimeric, i.e. recombinant, VLPs were precipitated by lysis of separate shake-flask culture samples, followed by PEG(polyethylene glycol)-treatment of the resulting cell lysates, according to the following protocol. 5 mL aliquots of each shake-flask culture were centrifuged to pellet the cells. Pelleted cells were resuspended in 0.1M phosphate buffer (preferably a combination of monobasic and dibasic potassium phosphate), pH 7.0, at a 2 volume buffer to 1 volume pellet ratio. Cells were then sonicated for 10 sec, 4 times, with 2 minutes resting on ice in between. During this sonication procedure, the cell lysate should clear somewhat. Following sonication, lysozyme was added to final concentration of 0.5 mg/mL. Lysozyme digestion was allowed to proceed for 30 min at room temperature.

The resulting treated lysates were then centrifuged for 5 min at 15000×G at 4° C. The resulting supernatants were removed and their volumes measured. To each supernatant, PEG6000 was added to a final concentration of 4%; followed by NaCl addition to a final concentration of 0.2M, and incubation on ice for 1 hr or overnight at 4° C. Then, these were centrifuged at 2000×G for 15 min at 4° C. Precipitated pellets were then resuspended in 1/10 initial supernatant volume of phosphate buffer and stored at 4° C.

1.H. Sucrose Gradient Centrifugation

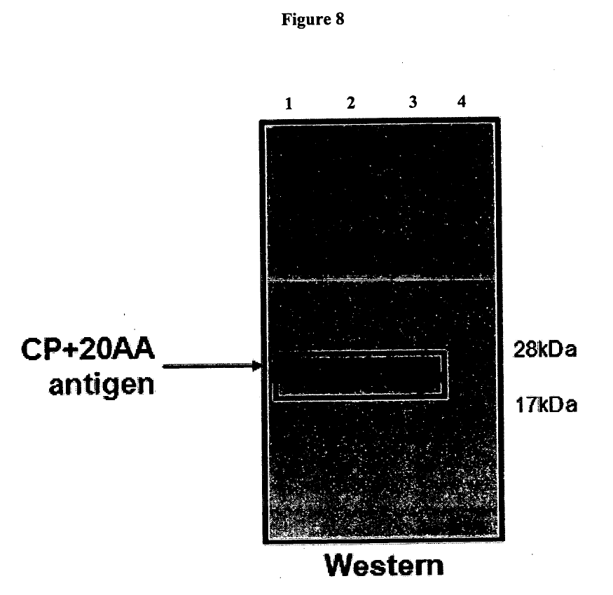

Sucrose solutions were made with sucrose (Sigma, Cat. S-5390) in phosphate buffer. Sucrose gradients were poured manually 10%, 20%, 30%, and 40% from top to bottom. The resuspended precipitated pellet samples were then spun in a Beckman-Coulter SW41-Ti rotor in a Beckman-Coulter Optima XL 100 K Ultracentrifuge for 1 hour with no braking. Each 1 mL fraction of the sucrose gradient was eluted separately and further spun down to obtain VLP pellets. VLP pellets were resuspended in phosphate buffer, electrophoresed on SDS-PAGE gels, and Western blotted using CCMV IgG as per the above protocol. Western blot was positive for VLP formation (FIG. 8). A portion of each resulting VLP preparation was used for electron microscopy.

1.I. Electron Microscopy Analysis

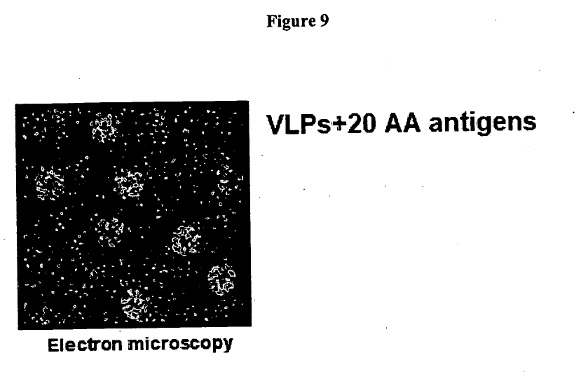

VLP samples were spotted on either collodion/carbon- or formvar/carbon-coated grids. Samples were stained with 2% phosphotungstic acid (PTA) and were imaged on a Philips CM-12 TEM transmission electron microscope (Serial #D769), operated at an accelerating voltage of 120 kV. Images were recorded digitally with a MultiScan CCD camera (from Gatan, Inc., Pleasanton, CA, USA; Model 749, Serial # 971119010). Formation of VLPs was verified (FIG. 9).

### Example 2

### Production of D2A21 AMP Trimers in CCMV VLPs in Pseudomonas and Recovery of AMPs Therefrom

2.A. Synthesis of D2A21 Insert:

A nucleotide sequence coding an anti-microbial peptide (“AMP”) trimer (“D2A21 trimer,” i.e. containing three D2A21 monomeric AMPs) was amplified out of plasmid pET-(D2A21)₃ using primers D2A21-BamHI-F (nucleic acid sequence: 5′-cgggatcctg ggacagcaaa tgggtcgcga tccg-3′ (SEQ ID NO:5)) and D2A21-BamHI-R (nucleic acid sequence: 5′-cgggatcccg tcgacggagc tcgaattcgg atcacc-3′ (SEQ ID NO:6)). PCR reactions were performed according to the same protocols as described in Example 1.A., above.

The resulting amplified insert contained a BamHI restriction site added at each end, for use in inserting the D2A21 trimer CDS into the CCMV129 CDS at the engineered BamHI site. The nucleotide sequence encoding, and the amino acid sequence of, the D2A21 trimer are shown in SEQ ID NOs:19 and 20, respectively.

Nucleotide sequence encoding the D2A21 trimer (SEQ ID NO:19):
5′-ttc gcg aag aag ttt gcg aaa aag ttc aag aaa ttt gcc aag aag ttt gcc aag ttc gca ttc
gcg ttc ggc gat ccg ttc gcg aag aag ttt gcg aaa aag ttc aag aaa ttt gcc aag aag ttt
gcc aag ttc gca ttc gcg ttc ggc gat ccg ttc gcg aag aag ttt gcg aaa aag ttc aag aaa
ttt gcc aag aag ttt gcc aag ttc gca ttc gcg ttc ggt-3′
Amino acid sequence of the D2A21 trimer (SEQ ID NO:20):
Phe Ala Lys Lys Phe Ala Lys Lys Phe Lys Lys Phe Ala Lys Lys Phe Ala Lys
Phe Ala Phe Ala Phe Gly Asp Pro Phe Ala Lys Lys Phe Ala Lys Lys Phe Lys
Lys Phe Ala Lys Lys Phe Ala Lys Phe Ala Phe Ala Phe Gly Asp Pro Phe Ala
Lys Lys Phe Ala Lys Lys Phe Lys Lys Phe Ala Lys Lys Phe Ala Lys Phe Ala
Phe Ala Phe Gly

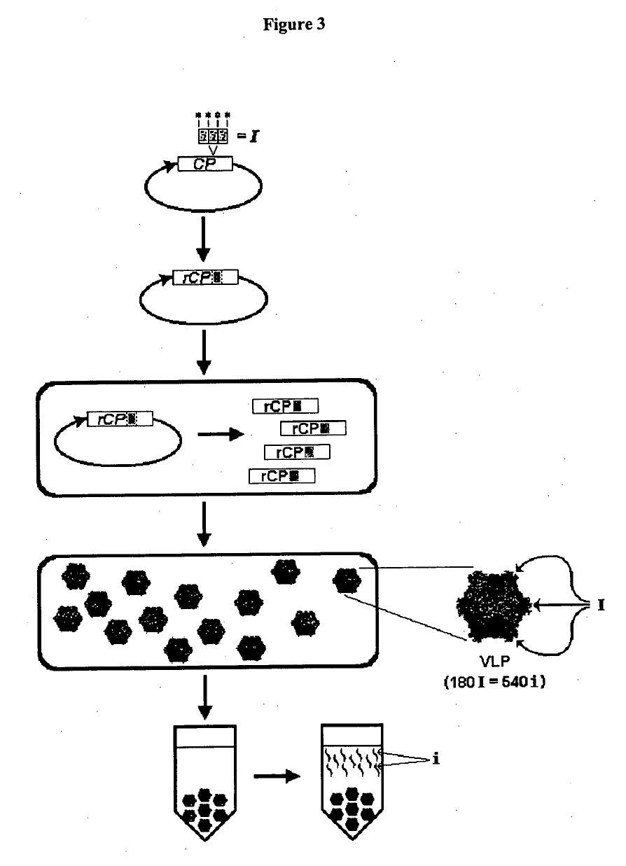

The trimer CDS contained the three AMP monomer CDSs separated by di-peptide Asp-Pro acid-labile cleavage site CDSs, as shown in FIG. 3. The entire trimer CDS was also bordered at each terminus by an dipeptide Asp-Pro acid-labile cleavage site CDS. The amplified insert was digested with BamHI restriction enzyme to create adhesive ends for cloning into the pESC-CCMV129BamHI shuttle plasmid at the BamHI site within the CCMV129 CDS. The resulting shuttle plasmid was digested with SpeI and XhoI restriction enzymes. The desired chimeric RBS/CDS fragment was isolated by gel purification.

2.B. Expression Plasmid Construction

The resulting chimeric CCMV129-(D2A21)3 polynucleotide was then inserted into the pMYC1803 expression plasmid in place of the buibui coding sequence, in operable attachment to the tac promoter. The resulting expression plasmid was screened by restriction digest with SpeI and XhoI for presence of the insert. The same protocols as described above for Example 1B were utilized.

### 2.C. Transformation and Expression

The resulting expression plasmid was transformed into P. fluorescens MB214, using the protocol described above for Example 1.C. Plate-colonized transformants were picked and transferred to shake flasks for expression, following the same protocol as described for Example 1.D., above.

2.D. Protein and VLP Recovery and Analysis

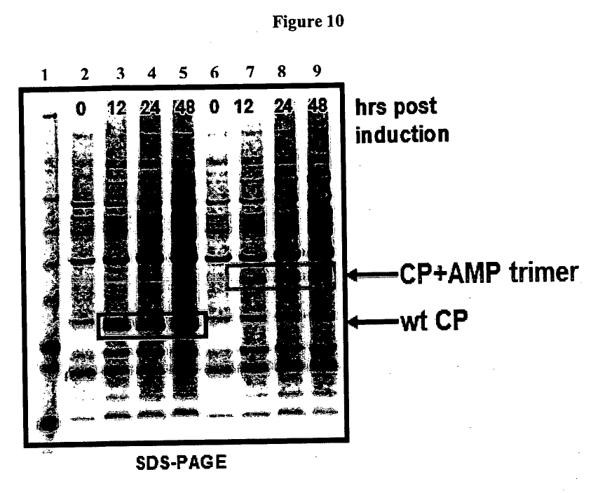

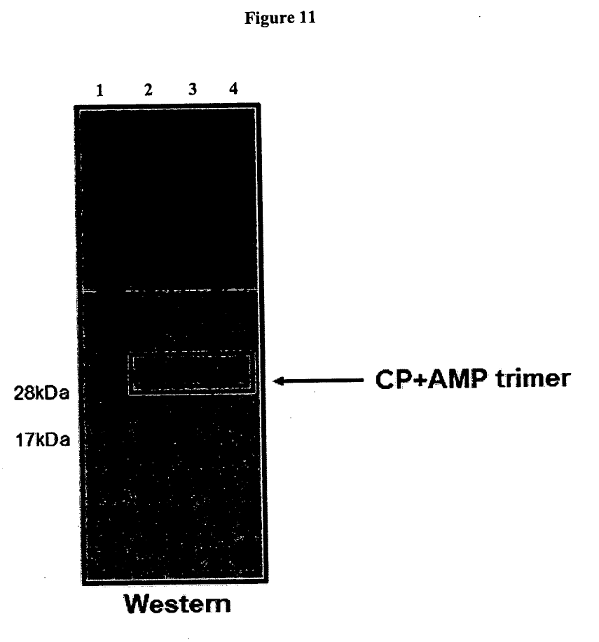

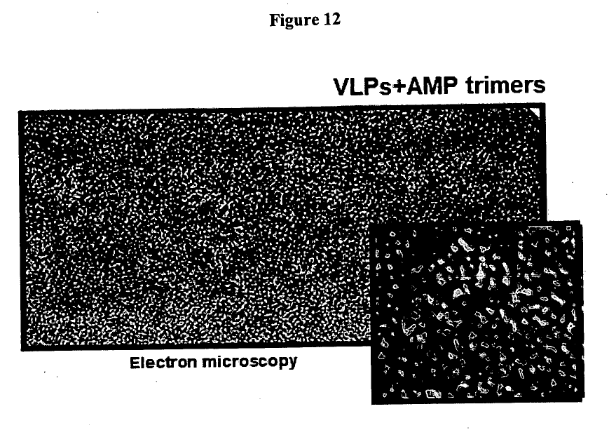

Shake-flask-cultured cells were lysed and fractionated, following the procedures of Example 1.E. The resulting fractions were analyzed by SDS-PAGE and Western blotting as described for Example 1.F. Chimeric VLPs were recovered by PEG precipitation and sucrose gradient centrifugation, and analyzed by electron microscopy, as described above for Examples 1.G. through 1.I. Chimeric CCMV VLP assembly was verified. Results were positive for the production of CCMV. SDS-PAGE for chimeric CP expression showed a 96 amino acids insert (FIG. 10), which was confirmed by western blot after fractionation on sucrose gradient to show VLP formation (FIG. 11) and by electron micrograph to verify VLP formation (FIG. 12).

2.E. Analysis of D2A21 Anti-Microbial Peptide Production

Soluble and insoluble protein fractions were further treated to characterize D2A21 peptides produced in the chimeric VLPs, as follows.

2.E.1. Acid Cleavage of D2A21

The insoluble fraction was dissolved in 15% v/v aqueous acetonitrile and approximately 40-50% v/v aqueous formic acid; the soluble fraction was resuspended in approximately 45-50% formic acid. The samples were then incubated at 60° C. for 24 hours to permit acid cleavage to proceed. The reactions were stopped by freezing to −20° C., at which temperature the treated samples were stored until HPLC analysis.

2.E.2. D2A21 Analysis by HPLC

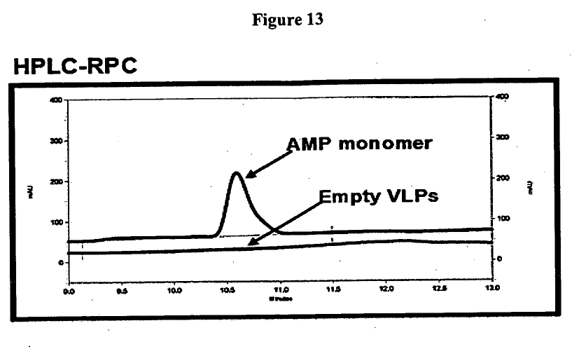

Soluble fractions were filtered through a 0.22 μm membrane; insoluble fractions were centrifuged to precipitate cellular debris and then filtered through a 0.22 μm membrane. 50 μL of each sample was added to 950 μL of 25% aqueous acetonitrile. A volume of 250 μL of each sample, containing a D2A21′ internal control peptide (10 μg control peptide total), was injected onto a VYDAC 250 mm reverse-phase C18 column, 6.4 mm internal diameter (available from Grace Vydac, Hesperia, Calif., USA), installed in a Beckman high performance liquid chromatography (HPLC) system. Elution was performed using an aqueous gradient of 25% acetonitrile/0.1% trifluoroacetic acid (TFA) to 75% acetonitrile/0.01% TFA over 30 minutes. Eluates were drip-collected into chromatography fractions. The appropriate peptide peak was observed only in the sample derived from VLPs containing the engineered peptide but not in the sample derived from non-engineered VLPs (FIG. 13).

2.E.3. Mass Spectrometry Analysis of D2A2] Peptides

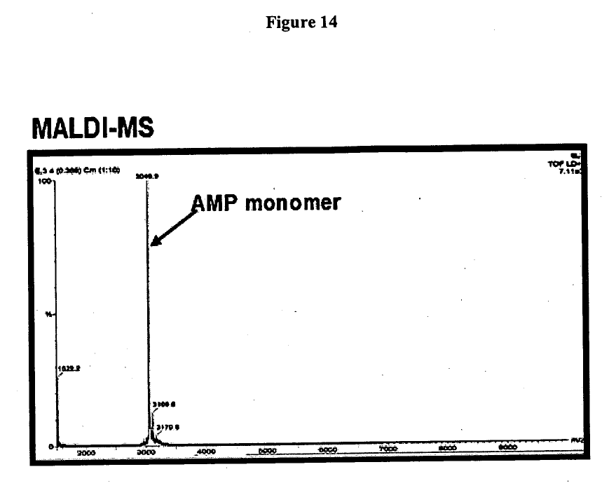

Mass spectrometry analysis of peptide controls and of chromatography fractions were performed using a Micromass M@LDI linear matrix-assisted laser desorption ionizationtime-of-flight (MALDI-TOF) mass spectrometry system (from Micromass UK Ltd., Manchester, UK). Before MS analysis, the HPLC fractions were concentrated by centrifugal evaporation, using a Speed Vac system (available from Thermo Savant, Milford, MA, USA; model 250DDA). The results demonstrated the accurate production of D2A21 AMPs and that the peptide that is released is the D2A21 peptide (FIG. 14). These results demonstrated that the use of a VLP fusion for peptide expression in Pseudomonads was effective avoided otherwise normal host-cell toxicity. Production of (A) chimeric VLPs has been demonstrated and production of (B) peptide multimers has been demonstrated in the VLP format (up to 96 amino acids total). Thus, this Example has:
(1) tested the ability of P. fluorescens to support CCMV CP expression and particle assembly,
(2) purified chimeric VLPs by a simple method (PEG precipitation),
(3) cleaved off the peptides of interest by previously tested methods (acid hydrolysis) and
(4) verified the peptide identity and integrity.

### Example 3

### Production of Anthrax Antigens in CCMV VLPs in Pseudomonas

3.A. Synthesis of PA Peptide Inserts

Four different Bacillus anthracis protective antigen (“PA”) peptides (PA1-PA4) were independently expressed in CCMV VLPs. Nucleic acids encoding PA1-PA4 were synthesized by SOE (splicing-by-overlap-extension) of synthetic oligonucleotides. The resulting nucleic acids contained BamHI recognition site termini. The nucleotide sequences encoding, and the amino acid sequences of, these PA peptides were respectively as follows: 1) for PA1, SEQ ID NOs:8 and 9; 2) for PA2, SEQ ID NOs:10 and 11; 3) for PA3, SEQ ID NOs:12 and 13; and 4) for PA4, SEQ ID NOs:14 and 15. The resulting nucleic acids were digested with BamHI to create adhesive ends for cloning into shuttle vector. Each of the resulting PA inserts was cloned in the pESC-CCMV129BamHI shuttle plasmid at the BamHI site of the CCMV129 CDS. Each resulting shuttle plasmid was digested with SpeI and XhoI restriction enzymes. Each of the desired chimeric CCMV129-PA-encoding fragments was isolated by gel purification.

| | |
|---|---|
| PA1 | |
| Nucleic Acid | 5′-agt aat tct cgt aag aaa cgt tct acc tct gct ggc cct acc gtg cct |
| Sequence (SEQ | gat cgt gat aat gat ggc att cct gat-3′ |
| ID NO:8) | |
| Amino Acid Sequence | Ser Asn Ser Arg Lys Lys Arg Ser Thr Ser Ala Gly Pro Thr |
| (SEQ ID NO:9) | Val Pro Asp Arg Asp Asn Asp Gly Ile Pro Asp |
| PA2 | |
| Nucleic Acid | 5′-agt cct gaa gct cgt cat cct ctc gtg gct gcg tat cct att gtg cat |
| Sequence (SEQ | gtt gat atg gaa aat att atc ctc tct-3′ |
| ID NO:10) | |
| Amino Acid Sequence | Ser Pro Glu Ala Arg His Pro Leu Val Ala Ala Tyr Pro Ile |
| (SEQ ID NO:11) | Val His Val Asp Met Glu Asn Ile Ile Leu Ser |
| PA3 | |
| Nucleic Acid | 5′-cgt att att ttc aat ggc aaa gat ctc aat ctc gtg gaa cgt cgt att |
| Sequence (SEQ | gct gct gtg aat cct tct gat cct ctc-3′ |
| ID NO:12) | |
| Amino Acid Sequence | Arg Ile Ile Phe Asn Gly Lys Asp Leu Asn Leu Val Glu Arg |
| (SEQ ID NO:13) | Arg Ile Ala Ala Val Asn Pro Ser Asp Pro Leu |
| PA4 | |
| Nucleic Acid | 5′-cgt caa gat ggc aaa acc ttc att gat ttc aaa aag tat aat gat aaa |
| Sequence (SEQ | ctc cct ctc tat att tct aat cct aat-3′ |
| ID NO:14) | |
| Amino Acid Sequence | Arg Gln Asp Gly Lys Thr Phe Be Asp Phe Lys Lys Tyr Asn |
| (SEQ ID NO:15) | Asp Lys Leu Pro Leu Tyr Ile Ser Asn Pro Asn |

3.B. Expression Plasmid Construction

The resulting chimeric CCMV129-PA polynucleotides were each then inserted into the pMYC1803 expression plasmid in place of the buibui coding sequence, in operable attachment to the tac promoter. The resulting expression plasmid was screened by restriction digest with SpeI and XhoI for presence of the insert. The same protocols as described above for Example 1B were utilized.

3.C. Transformation and Expression

The resulting expression plasmid was transformed into P. fluorescens MB214, using the protocol described above for Example 1.C. Plate-colonized transformants were picked and transferred to shake flasks for expression, following the same protocol as described for Example 1.D., above.

3.D. Protein and VLP Recovery and Analysis

Shake-flask-cultured cells were lysed and fractionated, following the procedures of Example 1.E. The resulting fractions were analyzed by SDS-PAGE and Western blotting as described for Example 1.F. Results were positive for the production of CCMV.

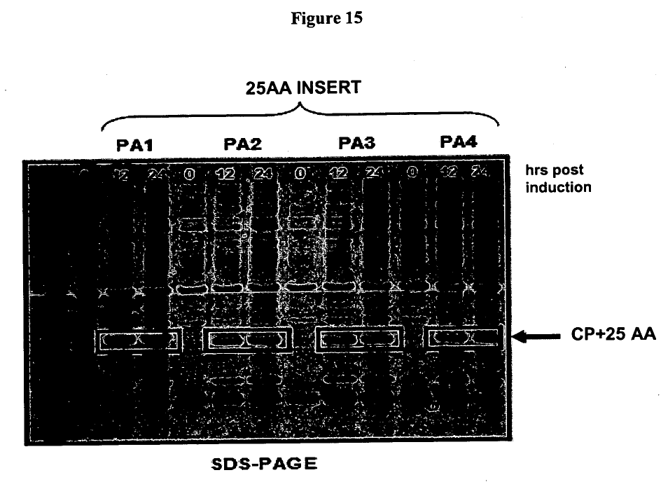

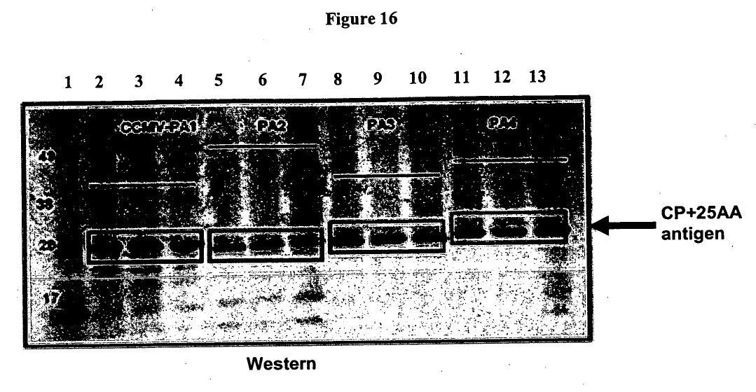

Results were positive for chimeric CCMV CP production (see FIG. 15). VLPs were recovered by PEG precipitation and sucrose gradient fractionation as described in the example 1.G. and 1.H. Western blot of sucrose gradient fraction was performed as described in 1.H. The results were positive for VLP formation (FIG. 16).

### Example 4

### Production of PBF20 AMP Monomers by Single and Double Insertion into CCMV VLPs in Psedomonas

The procedures set forth in Examples 1, 2, and 3 were followed. Nucleic acid encoding PBF20 monomeric peptides (encoding AMPs comprising the amino acid sequence 3-22 of amino acid sequence Asp Pro Lys Phe Ala Lys Lys Phe Ala Lys Lys Phe Ala Lys Lys Phe Ala Lys Lys Phe Ala Lys Asp Pro (SEQ ID NO:24)) and the acid cleavage sites comprising the amino acid sequence 1-2 and 23-24 of SEQ ID NO:24 was inserted individually into CCMV63-CP at AscI/NotI site and CCMV129-CP at BamHI site. The peptide was also inserted into R26C—CCMV63/129-CP both at the AscI/NotI site and BamHI site at the same time.

The resulting chimeric polynucleotides were each then inserted into the pMYC 1803 expression plasmid in place of the buibui coding sequence, in operable attachment to the tac promoter. The resulting expression plasmid was screened by restriction digest with SpeI and XhoI for presence of the insert and transformed into P. fluorescens MB214, using the protocol described above for Example 1.C. Plate-colonized transformants were picked and transferred to shake flasks for expression, following the same protocol as described for Example 1.D., above.

Shake-flask-cultured cells were lysed and fractionated, following the procedures of Example 1.E. The resulting fractions were analyzed by SDS-PAGE and Western blotting as described for Example 1.F. Results were positive for production of VLPs.

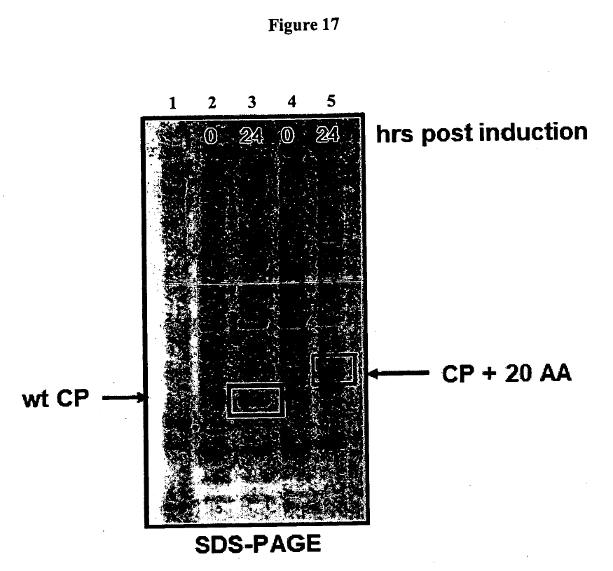

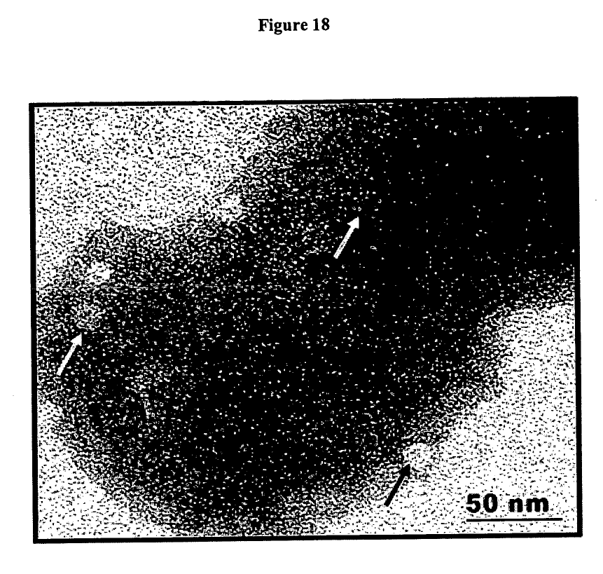

SDS-PAGE showing expression of chimeric CCMV63-CP engineered to express a 20 amino acid antimicrobial peptide PBF20 separated by acid hydrolysis sites in Pseudomonas fluorescens is shown in FIG. 17. The chimeric CCMV63-CP-PBF20 has slower mobility compared to the non-engineered wild type (wt) CCMV CP. Electron microscopy (EM) image of chimeric CCMV VLPs derived from CCMV63-CP and displaying a 20 amino acid antimicrobial peptide PBF20 separated by acid hydrolysis sites is shown in FIG. 18.

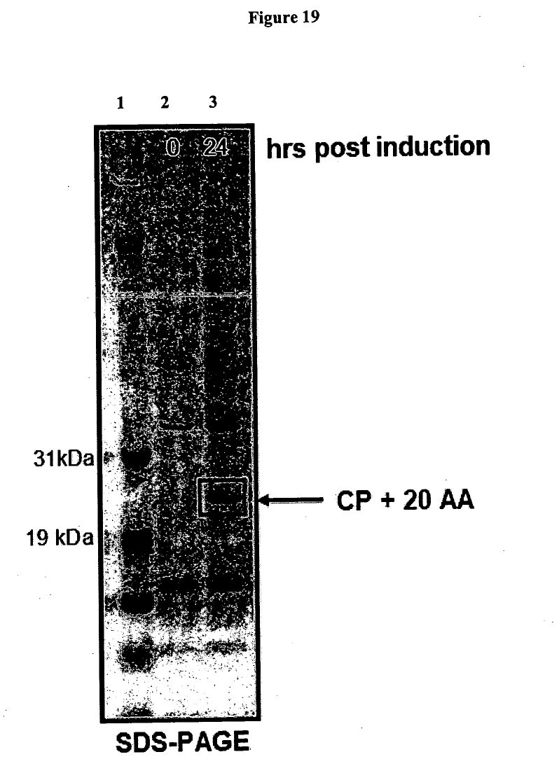

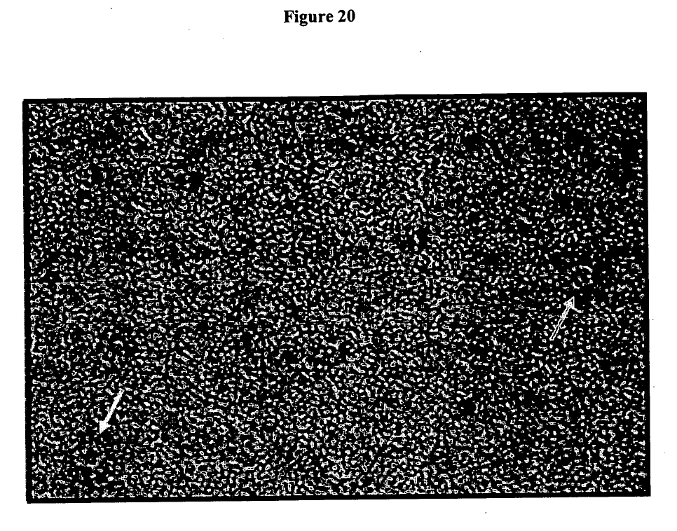

SDS-PAGE showing expression of chimeric CCMV129-CP engineered to express a 20 amino acid antimicrobial peptide PBF20 separated by acid hydrolysis sites in Pseudomonas fluorescens is shown in FIG. 19. The chimeric CCMV129-CP-PBF20 has slower mobility compared to the non-engineered wild type (wt) CCMV CP. Electron microscopy (EM) image of chimeric CCMV VLPs derived from CCMV129-CP and displaying a 20 amino acid antimicrobial peptide PBF20 separated by acid hydrolysis sites is shown in FIG. 20.

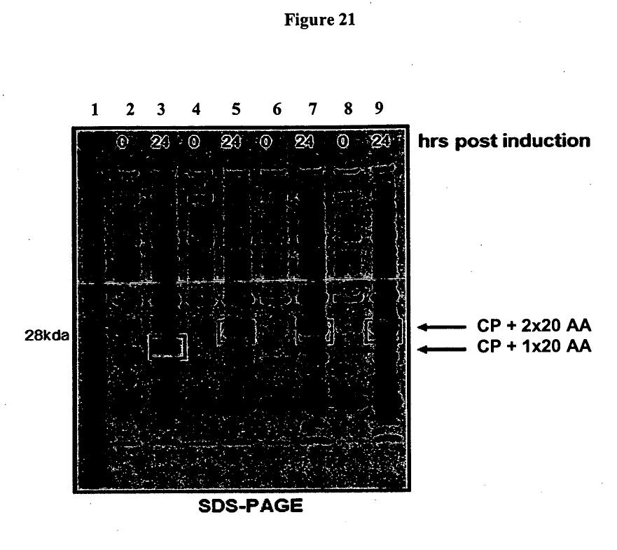

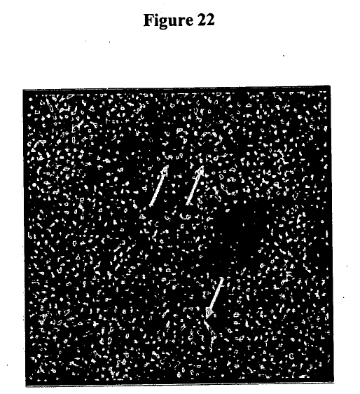

SDS-PAGE showing expression of chimeric CCMV63/129 CP engineered to express a 20 amino acid antimicrobial peptide PBF20 separated by acid hydrolysis sites in two different insertion sites in the CP in Pseudomonas fluorescens is shown in FIG. 21. Chimeric CP containing a double insert (CP+1×20 AA) has slower mobility on the SDS-PAGE gel compared to the capsid engineered to express a single insert (CP+1×20 AA) of the same peptide. Electron microscopy (EM) image of chimeric CCMV VLPs derived from CCMV63/129-CP displaying a 20 amino acid antimicrobial peptide PBF20 separated by acid hydrolysis sites in two insertion sites per capsid is shown in FIG. 22. Each VLP was found to contain up to 360 BPF20 monomers per particle.

### Example 5

### Production of Eastern Equine Encephalitis Virus (EEE) Antigens in CCMV VLPs in Pseudomonas

5.A. Synthesis of EEE Peptide Inserts

Two different EEE peptides (EEE-1 and EEE-2) were independently expressed in CCMV VLPs.

EEE-1 Peptide Sequence:

DLDTHFTQYKLARPYIADCPNCGHS (SEQ ID NO:25)

EEE-1 Nucleic Acid Sequence:

(SEQ ID NO:26)
5′gacctggacacccacttcacccagtacaagctggcccgcccgtacatcgccgactgcccgaactgcggccacagc-3′

EEE-2 Peptide Sequence:

GRLPRGEGDTFKGKLHVPFVPVKAK (SEQ ID NO:27)

EEE-2 Nucleic Acid Sequence:

(SEQ ID NO:28)
5′-ggccgcctgccgcgcggcgaaggcgacaccttcaagggcaagctgcacgtgccgttcgtgccggtgaaggccaag-3′

Nucleic acids encoding EEE-1 and EEE-2 were synthesized by SOE of synthetic oligonucleotides. The resulting nucleic acids contained BamHI recognition site termini. The sense and anti-sense oligonucleotide primers for synthesis of the inserts included the BamHI restriction sites and were as follows:

EEE1.S:

(SEQ ID NO:29)
5′ - cgg gga tcc tgg acc tgg aca ccc act tca ccc agt aca agc tgg ccc gcc cgt ac - 3′

EEE1.AS:

(SEQ ID NO:30)
5′ - cgc agg atc ccg ctg tgg ccg cag ttc ggg cag tcg gcg atg tac ggg cgg gcc agc - 3′

EEE2.S:

(SEQ ID NO:31)
5′ - cgg gga tcc tgg gcc gcc tgc cgc gcg gcg aag gcg aca cct tca agg gca agc - 3′

EEE2.AS:

(SEQ ID NO:32)
5′ - cgc agg atc ccc ttg gcc ttc acc ggc acg aac ggc acg tgc agc ttg ccc ttg - 3′

The resulting nucleic acids were digested with BamHI to create adhesive ends for cloning into the pESC—CCMV129BamHI shuttle plasmid.

Each of the resulting EEE inserts was cloned in the pESC—CCMV129BamHI shuttle plasmid at the BamHI site of the CCMV129 CDS. Each resulting shuttle plasmid was digested with SpeI and XhoI restriction enzymes. Each of the desired chimeric CCMV-129-EEE-encoding fragments was isolated by gel purification.

5.B. Expression Plasmid Construction

The resulting chimeric CCMV129-EEE polynucleotide fragments were each then inserted into the pMYC1803 expression plasmid restricted with SpeI and XhoI in place of the buibui coding sequence, in operable attachment to the tac promoter. The resulting expression plasmid was screened by restriction digest with SpeI and XhoI for presence of the insert. The same protocols as described above for Example 1.B. were utilized.

5.C. Transformation and Expression

The resulting expression plasmid is transformed into P. fluorescens MB214, using protocols described above in Example 1.C. The same protocols as described above for Example 1.D. are used for expression of chimeric VLPs displaying the EEE antigens.

5.D. Protein and VLP Recovery and Analysis

Shake-flask-cultured cells are lysed and fractionated, following the procedures of Example 1.E. The resulting fractions are analyzed by SDS-PAGE and Western blotting as described for Example 1.F.

1) A Pseudomonad cell that comprises a first nucleic acid construct comprising:
a) at least one nucleic acid sequence encoding a icosahedral viral capsid; and
b) at least one nucleic acid sequence encoding a recombinant peptide.

2) The cell of claim 1, wherein the Pseudomonad is Pseudomonas fluorescens.

3) The cell of claim 1, wherein the icosahedral viral capsid is from a virus that does not display a native tropism to a Pseudomonad cell.

4) The cell of claim 3, wherein the icosahedral viral capsid is from a plant icosahedral virus.

5) The cell of claim 4, wherein the plant icosahedral virus is selected from the group consisting of a Cowpea Chlorotic Mottle Virus, a Cowpea Mosaic Virus, and an Alfalfa Mosaic Virus.

6) The cell of claim 1, wherein the nucleic acid encodes at least two different icosahedral viral capsids.

7) The cell of claim 6, wherein at least one of the icosahedral viral capsids is from a plant icosahedral virus.

8) The cell of claim 1, wherein the nucleic acid encoding the recombinant peptide contains more than one monomer.

9) The cell of claim 8, wherein the nucleic acid encoding the recombinant peptide contains at least three monomers.

10) The cell of claim 8, wherein the monomers are operably linked as concatamers.

11) The cell of claim 1, wherein the recombinant peptide fused to the icosahedral capsid is a therapeutic peptide.

12) The cell of claim 1, wherein the recombinant peptide is an antigen.

13) The cell of claim 12, wherein the antigen is selected from the group consisting of a Canine Parvovirus antigen, a Bacillus Anthracis antigen, and an Eastern Equine Encephalitis viral antigen.

14) The cell of claim 1, wherein the recombinant peptide is an antimicrobial peptide.

15) The cell of claim 14, wherein the antimicrobial peptide is selected from the group consisting of D2A21 and PBF20.

16) The cell of claim 1, wherein the recombinant peptide is at least 7 amino acids in length.

17) The cell of claim 16, wherein the recombinant peptide is at least 15 amino acids in length.

18) The cell of claim 1, wherein the cell further comprises a second nucleic acid encoding a wild type icosahedral viral protein.

19) The cell of claim 1, wherein the cell further comprises a second nucleic acid comprising:
c) at least one nucleic acid sequence encoding a second icosahedral viral capsid; and
d) at least one nucleic acid sequence encoding a second recombinant peptide.

20) The cell of claim 19 wherein the first and second icosahedral viral capsids are different.

21) A Pseudomonad cell that comprises a fusion peptide, wherein the fusion peptide comprises:
a) at least one icosahedral viral capsid; and
b) at least one recombinant peptide.

22) The cell of claim 21 wherein the fusion peptide assembles within the cell to form a virus like particle.

23) The cell of claim 21 wherein the fusion peptide assembles within the cell to form a soluble cage structure.

24) The cell of claim 22, wherein the virus like particle is not capable of replication.

25) The cell of claim 22, wherein the virus like particle is not capable of infecting a cell.

26) The cell of claim 21, wherein the recombinant peptide is inserted into at least one surface loop of the icosahedral capsid.

27) The cell of claim 21, wherein a recombinant peptide is inserted into more than one surface loop of the icosahedral capsid.

28) The cell of claim 21, wherein the fusion peptide comprises more than one recombinant peptide fused to an icosahedral viral capsid.

29) The cell of claim 28, wherein the recombinant peptides are different.

30) The cell of claim 21, wherein the recombinant peptide is a therapeutic peptide.

31) The cell of claim 21, wherein the recombinant peptide is an antigen.

32) The cell of claim 31, wherein the antigen is selected from the group consisting of a Canine Parvovirus antigen, a Bacillus Anthracis antigen, and an Eastern Equine Encephalitis viral antigen.

33) The cell of claim 22, wherein the virus like particle is capable of use as a vaccine.

34) The cell of claim 21, wherein the recombinant peptide is a peptide that is an antimicrobial peptide.

35) The cell of claim 34, wherein the antimicrobial peptide is selected from the group consisting of D2A21 and PBF20.

36) The cell of claim 21, wherein the recombinant peptide is at least 7 amino acids in length.

37) The cell of claim 21, wherein the recombinant peptide is at least 15 amino acids in length.

38) The cell of claim 21, wherein the cell further comprises a wild type icosahedral viral capsid.

39) The cell of claim 21, wherein the cell further comprises a second fusion peptide comprising:
a) at least a second icosahedral viral capsid; and
b) at least a second recombinant peptide.

40) The cell of claim 39 wherein the second fusion peptide assembles within the cell to form a virus like particle or a soluble cage structure.

41) The cell of claim 39 wherein the second fusion peptide comprises a different amino acid sequence than the first fusion peptide.

42) The cell of claim 21 wherein the viral capsid and the recombinant peptide are linked by an amino acid sequence comprising a linker.

43) The cell of claim 42 wherein the linker amino acid sequence comprises a cleavable sequence.

44) The cell of claim 21, wherein the Pseudomonad is Pseudomonas fluorescens.

45) A nucleic acid construct comprising a first nucleic acid sequence encoding an icosahedral viral capsid operably linked to a second nucleic acid sequence encoding a peptide that is toxic to a microbial cell.

46) The construct of claim 45, wherein the icosahedral viral capsid is from a plant icosahedral virus.

47) The construct of claim 46, wherein the plant icosahedral virus is selected from the group consisting of a Cowpea Chlorotic Mottle Virus, a Cowpea Mosaic Virus, and an Alfalfa Mosaic Virus.

48) The construct of claim 46, wherein the toxic peptide comprises more than one peptide monomer sequence.

49) The construct of claim 46, wherein the toxic peptide comprises at least three peptide monomer sequences.

50) The construct of claim 48, wherein the monomers are operably linked to form a concatamer.

51) The construct of claim 45, wherein the operable linkage is internal to the first nucleic acid sequence encoding the capsid.

52) The construct of claim 45 wherein the second nucleic acid sequence encoding the toxic peptide is operably linked to the capsid sequence in a location encoding for at least one surface loop of the capsid.

53) The construct of claim 45, wherein the construct encodes more than one toxic peptide sequence operably linked to the capsid sequence locations encoding for more than one surface loop of the capsid.

54) The construct of claim 45, wherein the recombinant peptide is an antimicrobial peptide.

55) The construct of claim 54, wherein the antimicrobial peptide is selected from the group consisting of D2A21 and PBF20.

56) A process for producing a recombinant peptide comprising:
a) providing a Pseudomonad cell;
b) providing a nucleic acid encoding a fusion peptide, wherein the fusion peptide comprises at least one recombinant peptide and at least one icosahedral capsid;
c) expressing the nucleic acid in the Pseudomonad cell, wherein the fusion peptide assembles into virus like particles; and
d) isolating the virus like particles.

57) The process of claim 56, further comprising:
e) cleaving the fusion peptide to separate the recombinant peptide from the icosahedral viral capsid.

58) The process of claim 56, wherein the Pseudomonad is Pseudomonas fluorescens.

59) The process of claim 56, wherein the virus like particle is not capable of replication.

60) The process of claim 56, wherein the virus like particle is not capable of infecting a cell.

61) The process of claim 56, wherein the icosahedral viral capsid is from a virus that does not display a native tropism to a Pseudomonad cell.

62) The process of claim 56, wherein the icosahedral viral capsid is from a plant icosahedral virus.

63) The process of claim 62, wherein the plant icosahedral virus is selected from the group consisting of a Cowpea Chlorotic Mottle Virus, a Cowpea Mosaic Virus, and an Alfalfa Mosaic Virus.

64) The process of claim 56, wherein the nucleic acid comprises a nucleic acid sequence encoding at least two different icosahedral viral capsids.

65) The process of claim 64, wherein at least one of the icosahedral viral capsids is from a plant icosahedral virus.

66) The process of claim 56, wherein the recombinant peptide comprises more than one peptide monomer.

67) The process of claim 56, wherein the recombinant peptide comprises at least three monomers.

68) The process of claim 66, wherein the monomers are operably linked as a concatamer.

69) The process of claim 56, wherein the recombinant peptide is operably linked to at least one surface loop of the icosahedral capsid.

70) The process of claim 69, wherein a recombinant peptide is operably linked to more than one surface loop of the icosahedral capsid.

71) The process of claim 56, wherein the fusion peptide comprises more than one recombinant peptide, the recombinant peptides being dissimilar.

72) The process of claim 56, wherein the recombinant peptide is a therapeutic peptide.

73) The process of claim 56, wherein the recombinant peptide is an antigen.

74) The process of claim 73, wherein the antigen is selected from the group consisting of a Canine Parvovirus antigen, a Bacillus Anthracis antigen, and an Eastern Equine Encephalitis viral antigen.

75) The process of claim 56, wherein the virus like particle is capable of use as a vaccine.

76) The process of claim 56, wherein the recombinant peptide is a peptide that is an antimicrobial peptide.

77) The process of claim 76, wherein the antimicrobial peptide is selected from the group consisting of D2A21 and PBF20.

78) The process of claim 56, wherein the recombinant peptide is at least 7 amino acids in length.

79) The process of claim 56, wherein the recombinant peptide is at least 15 amino acids in length.

80) The process of claim 56, wherein the cell further comprises a second nucleic acid encoding a wild type icosahedral viral capsid.

81) The process of claim 56, wherein the cell further comprises a second nucleic acid encoding a second fusion peptide comprising:
a) at least a second icosahedral viral capsid; and
b) at least a second recombinant peptide.

82) The process of claim 81 comprising expressing the second nucleic acid in the cell.

83) The process of claim 81 wherein the second fusion peptide assembles within the cell to form a virus like particle or a soluble cage structure.

84) The process of claim 81 wherein the first icosahedral viral capsid comprises a first amino acid sequence and the second icosahedral viral capsid comprises a second amino acid sequence and the first and second capsid sequences are different.

85) The process of claim 81 wherein the first recombinant peptide comprises a first amino acid sequence and the second recombinant peptide comprises a second amino acid sequence and the first and second recombinant peptide sequences are different.


SEQUENCE LISTING (32 sequences)

SEQ ID NO: 1 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 2 - protein, 193 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNGILSKDVVAAMYPEAFKGITLEQLTADLTIYLYSSAALTEGDVIVHLEVEHVRPTFDDSFTPVY

SEQ ID NO: 3 - DNA, 33 bp, artificial
  CCMV-For
gactagtaggaggaaagagatgtctacagtcgg

SEQ ID NO: 4 - DNA, 27 bp, artificial
  CCMV-Rev
ccgctcgagtcattactaatacaccgg

SEQ ID NO: 5 - DNA, 34 bp, artificial
  D2A21-BamHI-F
cgggatcctgggacagcaaatgggtcgcgatccg

SEQ ID NO: 6 - DNA, 36 bp, artificial
  D2A21-BamHI-R
cgggatcccgtcgacggagctcgaattcggatcacc

SEQ ID NO: 7 - protein, 20 aa, Parvovirus H1
WACRGTAGWPPSGCTAPSGS

SEQ ID NO: 8 - DNA, 75 bp, Bacillus anthracis
agtaattctcgtaagaaacgttctacctctgctggccctaccgtgcctgatcgtgataatgatggcattcctgat

SEQ ID NO: 9 - protein, 25 aa, Bacillus anthracis
SNSRKKRSTSAGPTVPDRDNDGIPD

SEQ ID NO: 10 - DNA, 75 bp, Bacillus anthracis
agtcctgaagctcgtcatcctctcgtggctgcgtatcctattgtgcatgttgatatggaaaatattatcctctct

SEQ ID NO: 11 - protein, 25 aa, Bacillus anthracis
SPEARHPLVAAYPIVHVDMENIILS

SEQ ID NO: 12 - DNA, 75 bp, Bacillus anthracis
cgtattattttcaatggcaaagatctcaatctcgtggaacgtcgtattgctgctgtgaatccttctgatcctctc

SEQ ID NO: 13 - protein, 25 aa, Bacillus anthracis
RIIFNGKDLNLVERRIAAVNPSDPL

SEQ ID NO: 14 - DNA, 75 bp, Bacillus anthracis
cgtcaagatggcaaaaccttcattgatttcaaaaagtataatgataaactccctctctatatttctaatcctaat

SEQ ID NO: 15 - protein, 25 aa, Bacillus anthracis
RQDGKTFIDFKKYNDKLPLYISNPN

SEQ ID NO: 16 - DNA, 20 bp, artificial
  Parvo-BamHI-F
cgggatcctggacccggatg

SEQ ID NO: 17 - DNA, 21 bp, artificial
  Parvo-BamHI-R
cgggatccccgggtctctttc

SEQ ID NO: 18 - DNA, 60 bp, Parvovirus H1
tgggcctgccgcggcacggccggctggccgccgtccggctgcacggcgccgtccgggtcg

SEQ ID NO: 19 - DNA, 228 bp, artificial
  D2A21 trimer
ttcgcgaagaagtttgcgaaaaagttcaagaaatttgccaagaagtttgccaagttcgcattcgcgttcggcgatccgttcgcgaagaagtttgcgaaaaagttcaagaaatttgccaagaagtttgccaagttcgcattcgcgttcggcgatccgttcgcgaagaagtttgcgaaaaagttcaagaaatttgccaagaagtttgccaagttcgcattcgcgttcggt

SEQ ID NO: 20 - protein, 76 aa, artificial
  D2A21 trimer
FAKKFAKKFKKFAKKFAKFAFAFGDPFAKKFAKKFKKFAKKFAKFAFAFGDPFAKKFAKKFKKFAKKFAKFAFAFG

SEQ ID NO: 21 - DNA, 573 bp, Cowpea chlorotic mottle virus
atgtctacagtcggaacagggaagttaactcgtgcacaacgaagggctgcggcccgtaagaacaagcggaacactcgtgtggtccaacctgttattgtagaacccatcgcttcaggccaaggcaaggctattaaagcatggaccggttacagcgtatcgaagtggaccgcctcttgcgcggccgccgaagctaaagtaacctcggctataactatctctctccctaatgagctatcgtccgaaaggaacaagcagctcaaggtaggtagagttttattatggcttgggttgcttcccagtgttagtggcacagtgaaatcctgtgttacagagacgcagactactgctgctgcctcctttcaggtggcattagctgtggccgacaactcgaaagatgttgtcgctgctatgtaccccgaggcgtttaagggtataacccttgaacaactcaccgcggatttaacgatctacttgtacagcagtgcggctctcactgagggcgacgtcatcgtgcatttggaggttgagcatgtcagacctacgtttgacgactctttcactccggtgtattag

SEQ ID NO: 22 - DNA, 597 bp, artificial
  CCMV63-CP ORF
atgtctacagtcggaacagggaagttaactcgtgcacaacgaagggctgcggcccgtaagaacaagcggaacacttgtgtggtccaacctgttattgtagaacccatcgcttcaggccaaggcaaggctattaaagcatggaccggttacagcgtatcgaagtggaccgcctcttgtgcggctgccgaagcttggcgcgccgcggccgctaaagtaacctcggctataactatctctctccctaatgagctatcgtccgaaaggaacaagcagctcaaggtaggtagagttttattatggcttgggttgcttcccagtgttagtggcacagtgaaatcctgtgttacagagacgcagactactgctgctgcctcctttcaggtggcattagctgtggccgacaactcgaaagatgttgtcgctgctatgtaccccgaggcgtttaagggtataacccttgaacaactcaccgcggatttaacgatctacttgtacagcagtgcggctctcactgagggcgacgtcatcgtgcatttggaggttgagcatgtcagacctacgtttgacgactctttcactccggtgtattagtaatga

SEQ ID NO: 23 - DNA, 606 bp, artificial
  R26C-CCMV63/129-CP ORF
atgtctacagtcggaacagggaagttaactcgtgcacaacgaagggctgcggcccgtaagaacaagcggaacacttgtgtggtccaacctgttattgtagaacccatcgcttcaggccaaggcaaggctattaaagcatggaccggttacagcgtatcgaagtggaccgcctcttgtgcggctgccgaagcttggcgcgccgcggccgctaaagtaacctcggctataactatctctctccctaatgagctatcgtccgaaaggaacaagcagctcaaggtaggtagagttttattatggcttgggttgcttcccagtgttagtggcacagtgaaatcctgtgttacagagacgcagactactgctgctgcctcctttcaggtggcattagctgtggccgacaacgggatcctgtcgaaagatgttgtcgctgctatgtaccccgaggcgtttaagggtataacccttgaacaactcaccgcggatttaacgatctacttgtacagcagtgcggctctcactgagggcgacgtcatcgtgcatttggaggttgagcatgtcagacctacgtttgacgactctttcactccggtgtattagtaatga

SEQ ID NO: 24 - protein, 24 aa, artificial
  PBF20 AMP Monomer
DPKFAKKFAKKFAKKFAKKFAKDP

SEQ ID NO: 25 - protein, 25 aa, Eastern equine encephalomyelitis virus
DLDTHFTQYKLARPYIADCPNCGHS

SEQ ID NO: 26 - DNA, 75 bp, Eastern equine encephalomyelitis virus
gacctggacacccacttcacccagtacaagctggcccgcccgtacatcgccgactgcccgaactgcggccacagc

SEQ ID NO: 27 - protein, 25 aa, Eastern equine encephalomyelitis virus
GRLPRGEGDTFKGKLHVPFVPVKAK

SEQ ID NO: 28 - DNA, 75 bp, Eastern equine encephalomyelitis virus
ggccgcctgccgcgcggcgaaggcgacaccttcaagggcaagctgcacgtgccgttcgtgccggtgaaggccaag

SEQ ID NO: 29 - DNA, 56 bp, artificial
  EEE1.S
cggggatcctggacctggacacccacttcacccagtacaagctggcccgcccgtac

SEQ ID NO: 30 - DNA, 57 bp, artificial
  EEE1.AS
cgcaggatcccgctgtggccgcagttcgggcagtcggcgatgtacgggcgggccagc

SEQ ID NO: 31 - DNA, 54 bp, artificial
  EEE2.S
cggggatcctgggccgcctgccgcgcggcgaaggcgacaccttcaagggcaagc

SEQ ID NO: 32 - DNA, 54 bp, artificial
  EEE2.AS
cgcaggatccccttggccttcaccggcacgaacggcacgtgcagcttgcccttg